(19) United States
(12) Patent Application Publication
(10) Pub. No.: US 2026/0355424 A1
(43) Pub. Date: Nov. 12, 2026

(54) ASSEMBLY FOR A DRUG DELIVERY DEVICE

(71) Applicant: Sanofi, PARIS (FR)

(72) Inventors: Simon Francis Brereton, Melbourn (GB); Louise Hodgson, Melbourn (GB); Thomas Mark Kemp, Melbourn (GB); Kevin Ranucci, Bridgewater, NJ (US); Hugo Revellat, Melbourn (GB); William Timmis, Melbourn (GB)

(21) Appl. No.: 19/125,469

(22) PCT Filed: Oct. 31, 2023
(86) PCT No.: PCT/EP2023/080364

§ 371 Date: Apr. 29, 2025

(30) Foreign Application Priority Data
Oct. 31, 2022 (EP) 22315254.7

(51) Int. Cl. A61M 5/32; A61M 5/20

(52) U.S. Cl. CPC A61M 5/3245; A61M 5/20; A61M 5/3202; A61M 2005/2013; A61M 2005/208; A61M 2005/3247; A61M 2202/0007; A61M 2205/583

(57) ABSTRACT

An assembly for a drug delivery device comprising a device body, having a proximal end and a distal end, a needle cover, wherein the needle cover is movable relative to the device body along a longitudinal axis, and a needle cover lock mechanism, comprising at least one blocking element, the blocking element defining a blocking surface, at least one flexible arm, having a stop surface, wherein the needle cover lock mechanism is configured to have a locked state, wherein the stop surface and the blocking surface are arranged to cooperate to block movement of the needle cover in a proximal direction along the longitudinal axis in the locked state of the needle cover lock mechanism, and wherein the flexible arm comprises at least one protrusion which is positioned distally from the stop surface.

### CROSS REFERENCE TO RELATED APPLICATIONS

The present application is the national stage entry of International Patent Application No. PCT/EP2023/080364, filed on Oct. 31, 2023, and claims priority to Application No. EP 22315254.7, filed on Oct. 31, 2022, the disclosures of which are incorporated herein by reference.

### BACKGROUND

Due to increasing demand, hand-held drug delivery devices require mass production. Reducing manufacturing costs and increasing manufacturing speed is an ongoing challenge. By reducing the number of components in a device, both manufacturing speed can be increased and manufacturing costs can be reduced. However, a reduction in the number of components must be viewed critically. For example, the absence of injury protection components for the user can lead to serious disadvantages.

### SUMMARY

The present disclosure relates to facilitating improvements associated with drug delivery devices, particularly with regard to the simplification of the design, while taking into account the safety of the user.

One aspect of the present disclosure relates to an assembly for a drug delivery device. The drug delivery device may comprise the assembly, which is disclosed in the following. The drug delivery device may be provided to dispense drug or medicament. The drug delivery device may be a hand-held drug delivery device. The drug delivery device may be an autoinjector. The drug delivery device may have a drive energy source, e.g. a drive spring or another type of energy source such as a gas reservoir, for providing energy for a drug delivery operation. The drug delivery device is configured to perform a drug delivery operation, e.g. using energies obtainable from the drive energy sources. The assembly comprises a housing or device body having a proximal end and a distal end. The device body may form the outer surface of the assembly and/or the drug delivery device and may delimit the assembly and/or the drug delivery device from the periphery. The periphery may be anything that is external to the device body and not physically connected to the device body. The device body may be held directly by the user's hands when using the drug delivery device. The drug delivery device may have a medicament container for receiving the drug and a needle, being associated with the respective medicament container. The container may be prefilled with the drug. The needles are expediently configured to pierce a skin of a user. Through the needles, the drug may be administered to the user, e.g. into the user's tissue. The energies of the drive energy sources may be used to drive members, e.g. plunger and plunger rods, of the drug delivery device in order to dispense drug from the medicament containers. For the drug delivery operation, the drive members may be displaced in a distal direction relative to the device body by the energies provided by the drive energy sources. Furthermore the assembly comprises a needle cover. The needle cover is movable relative to the device body along a longitudinal axis of the device body. The device body can be a body that encloses the components of the drug delivery device, such as the needle cover, a needle cover spring, optionally a syringe holder, a pre-filled syringe i.e. the medicament container, the plunger, the drive spring, a drive spring holder and an audible indicator such as a clicker. The needle cover may be at least partially arranged inside of the device body. The assembly may be configured such that the needle cover cannot rotate relative to the device body, or vice versa. The needle cover comprises a needle cover lock mechanism. The needle cover lock mechanism comprises at least one blocking element, such as, for example, a ramp-like element. The blocking element may be located on the inner circumferential surface of the device body. The blocking element may be an integral part of the device body. The blocking element defines a blocking surface. The blocking surface may face the distal end of the device body. The needle cover lock mechanism comprises at least one flexible arm having a stop surface. The flexible arm comprises at least one protrusion which is positioned distally from the stop surface. The needle cover lock mechanism is configured to have a locked state. The stop surface and the blocking surface are arranged to cooperate to block movement of the needle cover in the proximal direction along the longitudinal axis in the locked state of the needle cover lock mechanism. In the locked state the blocking surface may abut the stop surface.

The design of the flexible arm and blocking element provides simple structured and cost effective components that provide a reliable and therefore safe locking mechanism, to prevent unintended axial movement of the needle cover relative to the device body. The needle cover can thus be arranged in a position in which it is axially distal from a needle tip and encloses the needle in a manner that substantially reduces the risk of injury to the user from the needle tip. Furthermore, the needle cover can thus be arranged in a position that favors the assembly of the drug delivery device and thus facilitates a faster production of the device.

In an embodiment the angular extension of the stop surface is bigger than the angular extension of the protrusion. In the context of the present disclosure, the angular extension of an element may be the length of the element along the perimeter.

In an embodiment the length of the protrusion along the longitudinal axis is shorter than the length of the flexible arm.

In an embodiment the protrusion has a chamfer at its distal end region.

In an embodiment the proximal end region of the protrusion is directly connected to the stop surface.

In an embodiment the protrusion and the stop surface are an integral component.

In an embodiment the angular extension of the stop surface is bigger than the angular extension of the blocking surface. Alternatively, the angular extension of the stop surface corresponds to the angular extension of the blocking surface.

In an embodiment the angular extension of the protrusion is smaller than the angular extension of the blocking surface.

In an embodiment the needle cover is movable between an initial position and an intermediate position relative to the device body along the longitudinal axis. The needle cover lock mechanism may be in the locked state when the needle cover is in the initial position and in an unlocked state when the needle cover is in the intermediate position. In the context of the present disclosure, the initial position may also be referred to as the third cover position and the intermediate position may also be referred to as the first cover position. In the initial position the needle cover may be arranged more distally relative to the device body along the longitudinal axis than in the intermediate position.

In an embodiment the assembly is configured such that the blocking element and the flexible arm overlap radially during a movement from the initial position to the intermediate position.

In an embodiment the assembly is configured such that the blocking element and the flexible arm overlap radially during a movement from the intermediate position to the initial position.

In an embodiment the assembly is configured such that the flexible arm is moved radially inward when the radial overlap between the blocking element and the flexible arm begins during the movement from the intermediate position to the initial position.

In an embodiment the assembly is configured such that the flexible arm is moved radially inwardly during the movement of the needle cover from the intermediate position to the initial position.

In an embodiment the assembly is configured such that the flexible arm is moved radially outward when the radial overlap between the blocking element and the flexible arm ends during the movement from the initial position to the intermediate position.

In an embodiment the blocking element comprises a blocking element recess. The blocking element recess may extend along the longitudinal axis of the inner circumferential surface of the device body. The blocking element recess may be configured such that a twisting of the flexible arm is prevented when the blocking element and the flexible arm the radial overlap.

In an embodiment the needle cover lock mechanism is configured such that the protrusion is guided in the blocking element recess during movement from the initial position to the intermediate position and/or during movement from the intermediate position to the initial position.

In an embodiment the blocking element, defines two blocking surfaces separated from each other. The two a blocking surfaces may be identical in shape. The two blocking surfaces may have the same axial position. The two blocking surfaces may be angularly offset from each other, and wherein the two blocking surfaces have the same axial position relative to the device body.

In an embodiment the angular extension of the stop surface, corresponds to that of the angular extension of the two blocking surfaces and the blocking element recess together.

In an embodiment the blocking element has a ramp shape having a radially inward inclination in the distal direction. The blocking element may protrude radially inwardly from an inner surface of the device body.

In an embodiment the blocking element is intersected by the blocking element recess in the longitudinal direction. The blocking element may consist of two ramps arranged side by side along the circumference of the inner surface of the device body.

In an embodiment blocking element is distally offset from a drug window of the device body. The blocking element may be aligned with the drug window in the circumferential direction.

In an embodiment the flexible arm comprises a hinge. The protrusion may be axially positioned between the hinge and the stop surface. The protrusion, the stop surface and the hinge may be an integral part of the flexible arm. In the present disclosure, integral means that a component or part is a single piece. The flexible arm may be an integral part of the needle cover.

In an embodiment the protrusion and/or the stop surface extend radially outward from the flexible arm. In the present disclosure, radially outward corresponds to the radial direction pointing away from the longitudinal axis, wherein radially inward corresponds to the radial direction pointing to the longitudinal axis.

In an embodiment the stop surface can be a cuboidal elevation of the flexible arm. The protrusion may be a web. The hinge may be a circular recess.

In an embodiment, at its proximal end region, the protrusion or web can merge with the stop surface in the longitudinal direction. The protrusion or web may be joined to the stop surface by a T-joint.

In an embodiment the angular extension of the protrusion corresponds to the angular extension of the blocking element recess. Alternatively the angular extension of the protrusion may be smaller than the angular extension of the blocking element recess.

In an embodiment the assembly comprises a cap. The assembly may be configured such that the flexible arm is pressed radially inward when the cap is joined to the device body. Moreover, the assembly may be configured such that an axial movement of the cap in the proximal direction relative to the device body brings a portion of the cap into direct contact with the protrusion so that the protrusion is moved radially inward to shift the needle cover lock mechanism from the locked state to the unlocked state. The needle cover may be movable relative to the device body along the longitudinal axis in proximal direction in the unlocked state.

In an embodiment the assembly comprises two needle cover lock mechanisms which are arranged opposite to each other on the same axial position. The two needle cover lock mechanisms may be arranged on the same axial position with an angular offset, e.g. 180 degrees. Each of the two needle cover lock mechanisms may have any of the features described above for the needle cover lock mechanism. Specifically, the needle cover may have two opposing flexible arms with the features described above. Furthermore, the device body may have two opposing blocking elements with the features described above. Accordingly, the device body may have, for example, four blocking surfaces.

In an embodiment a pair of the blocking surfaces are arranged such that an angle between them is less than 90 degrees, for example, less than 45 degrees in a circumferential direction.

In an embodiment the two pairs of the blocking surfaces are angularly offset from each other, e.g. by 180 degrees.

In the present invention, singular expressions such as “a flexible arm”, “a blocking surface”, etc. are used for ease of reading the description and claims. Since the assembly according to the invention “comprises” or “has” components or features, respectively, however, such a singular expression does not limit the number of components or features concerned. Rather, such a singular expression is intended to be understood as “at least one flexible arm”, “at least one blocking surface”, etc., unless the context indicates otherwise.

According to a further aspect, a method of delivering a drug from a drug delivery device is provided, the method comprising using the drug delivery device according to the present disclosure, e.g. according to any one of the above-mentioned embodiments.

According to a further aspect, a drug for use in a method of treating a patient is provided, wherein the method comprises delivering the drug to the patient using the drug delivery device according to the present disclosure, e.g. according to any one of the above-mentioned embodiments.

The making and using of the present embodiments are discussed in detail below. It should be appreciated, however, that the present disclosure provides many applicable concepts that can be embodied in a wide variety of specific contexts. The specific embodiments discussed are merely illustrative of specific ways to make and use the disclosed concepts, and do not limit the scope of the claims.

Moreover, same reference numerals refer to same technical features if not stated otherwise. As far as “may” is used in this application it means the possibility of doing so as well as the actual technical implementation. The present concepts of the present disclosure will be described with respect to embodiments below in a more specific context namely drug delivery devices, especially drug delivery devices for humans or animals. The disclosed concepts may also be applied, however, to other situations and/or arrangements as well, e.g. for other injectors, spraying devices or inhalation devices.

The foregoing has outlined rather broadly the features and technical advantages of embodiments of the present disclosure. Additional features and advantages of embodiments of the present disclosure will be described hereinafter, e.g. of the subject-matter of dependent claims. It should be appreciated by those skilled in the art that the conception and specific embodiments disclosed may be readily utilized as a basis for modifying or designing other structures or processes for realizing concepts which have the same or similar purposes as the concepts specifically discussed herein. It should also be recognized by those skilled in the art that equivalent constructions do not depart from the spirit and scope of the disclosure, such as defined in the appended claims.

### BRIEF DESCRIPTION OF THE FIGURES

For a more complete understanding of the presently disclosed concepts and the advantages thereof, reference is now made to the following description in conjunction with the accompanying drawings. The drawings are not drawn to scale. In the drawings the following is illustrated in:

FIGS. 1A to 1D depict a cross section of a drug delivery device according to a first embodiment and in different operating states.

FIG. 2 depicts an exploded view of an example of a drug delivery device without or with optional separate syringe holder.

FIGS. 3A to 3I depict an optional cap and optional cap lid.

FIGS. 4A and 4B depict a perspective view of an optional grabber and a cross section of the grabber, respectively.

FIG. 4C depicts an exemplary embodiment of a single piece of a sheet which can form the grabber carrier.

FIGS. 4D and 4E depict the grabber in an engaged position with the needle shield of the syringe.

FIG. 4F depicts a cutout view of the grabber of the previous embodiment assembled within a cap.

FIG. 4G depicts the interaction between the grabber retention bosses, e.g. the boss of a cap and the opening of an exemplary grabber in detail.

FIG. 4H depicts a cross section of a frontal end of an injection device with a mounted cap and a grabber mounted thereon and interacting with the needle shield.

FIG. 5 depicts an optional needle cover (needle shroud).

FIG. 6A depicts a needle cover spring.

FIG. 6B depicts a cross-sectional view of the needle cover spring assembled in the drug delivery device, in a pre-use state of the drug delivery device.

FIG. 7A depicts a device body.

FIG. 7B depicts a cross-sectional view of the device body.

FIG. 7C depicts a perspective cross-sectional view of a distal end of the device body.

FIG. 7D depicts a cross-sectional view of a central part of the device body with a syringe holder.

FIG. 7E depicts a perspective view of a syringe holder front stop of the device body.

FIG. 7F depicts a cross-sectional view of a distal end of the device body with a needle cover in the third cover position.

FIG. 7G depicts a perspective view of a needle cover lock structure interacting with a flexible arm of the needle cover.

FIG. 8A depicts an optional syringe holder.

FIG. 8B depicts a perspective view of an optional syringe holder.

FIG. 8C depicts a detailed view of a further exemplary embodiment of the flexible holder arms.

FIG. 8D depicts an optional syringe holder comprising the flexible holder arms of FIG. 8C.

FIG. 9 depicts an optional pre-filled syringe.

FIG. 10 depicts a plunger.

FIG. 10A depicts a plunger release mechanism in a first state.

FIG. 10B depicts the plunger release mechanism in a second state.

FIG. 10C depicts the plunger release mechanism during assembly of the drive subassembly.

FIG. 10D depicts the plunger release mechanism during the final assembly.

FIG. 10E depicts a further state of the plunger release mechanism.

FIG. 10F depicts a schematic view of the plunger release mechanism after depression of the sleeve into the retracted position.

FIG. 10G depicts a schematic detail view of the plunger release mechanism after final assembly and prior to depression of the sleeve.

FIG. 10H depicts a schematic detail view of the plunger release mechanism during depression of the sleeve.

FIG. 10I depicts a longitudinal rib at the inside of a rigid arm of drive spring holder.

FIG. 10J depicts a perspective view of a plunger according to a second embodiment.

FIG. 10K depicts a distal view of the plunger according to the second embodiment.

FIG. 10L depicts a cross section along a radial direction through the shaft of the plunger according to the second embodiment.

FIG. 10M depicts a cross section along a longitudinal direction through the shaft of the plunger.

FIG. 11A depicts a drive spring according to an embodiment of the disclosure,

FIG. 11B depicts the drive spring of FIG. 11A assembled in the drug delivery device during the actuation of the plunger.

FIG. 11C depicts the drive spring of FIGS. 11A and 11B assembled in the drug delivery device before the actuation of the plunger.

FIGS. 12A and 12B depict perspective views of the drive spring holder.

FIG. 12C depicts a syringe backstop mechanism.

FIG. 12D depicts a cross-sectional view of a proximal part of the drive spring holder.

FIGS. 12E to 12G depict different embodiments of the flexible portions of the drive spring holder.

FIG. 13A depicts an optional audible indicator (clicker).

FIG. 13B depicts an indicator holder, exemplarily comprised on a drive spring holder,

FIG. 13C depicts a perspective view of a supporting structure on the distal end of a flexible support arm.

FIG. 13D depicts a perspective view of a guiding structure of the indicator holder.

FIG. 13E depicts a cross section through the longitudinal symmetry axis of the indicator holder.

FIG. 13F depicts a rear sub-assembly (RSA) after assembling of the audible indicator but prior to priming of the audible indicator.

FIG. 13G depicts the rear sub-assembly (RSA) after priming of the audible indicator, for example, using a priming tool.

FIG. 13H depicts the priming tool.

FIG. 13I depicts a state during final assembling of the RSA and front sub-assembly (FSA) shortly before priming of the audible indicator.

FIGS. 14A to 14J depict an assembly steps for the optional syringe holder and pre-filled syringe into the device.

FIG. 15A depicts a flow diagram of an exemplary feedback order during use of the drug delivery device.

FIGS. 15B to 15D show different views from the drug window during a dose dispensing process.

FIGS. 16 and 17 depict a rear sub-assembly and a front sub-assembly of the drug delivery device.

FIG. 18 depicts an expanded structural formula, molecular formula, and molecular weight of fitusiran, e.g. sodium form.

### DETAILED DESCRIPTION

As a general note, “distal” is used herein to specify directions, ends or surfaces which are arranged or are to be arranged to face or point towards a dispensing end of the drug delivery device and/or point away from, are to be arranged to face away from or face away from the proximal end. On the other hand, “proximal” is used to specify directions, ends or surfaces which are arranged or are to be arranged to face away from or point away from the dispensing end and/or from the distal end of the drug delivery device or components thereof. The distal end may be the end closest to the dispensing end and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be the needle end where a needle is arranged or a needle or needle unit is or is to be mounted to the device, for example. “Axial” may be used synonymously with “longitudinal”.

The distal end DE may be an end that is closer to a needle compared to a proximal end PE. Certain embodiments in this disclosure are illustrated with respect to an injection device, e.g. an autoinjector. The device may comprise an advanced needle cover used as an activation element.

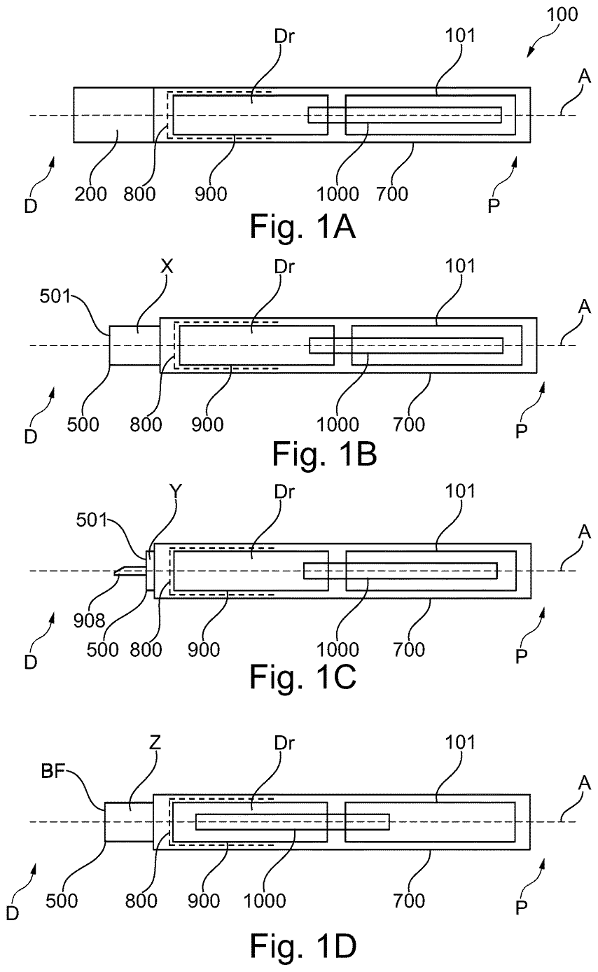

### 1. General Description of a Drug Delivery Device (FIGS. 1A to 1D)

FIGS. 1A through 1D illustrate an embodiment of a drug delivery device 100. Device 100 may be suitable as the device in the drug delivery arrangements described further above and below. The figures show device 100 in different states during its operation.

FIG. 1A illustrates the drug delivery device 100 in an initial or as delivered state. The drug delivery device 100 may comprise a housing or a device body 700. The device body 700 may be provided to retain and/or may retain a medicament container, for example a pre-filled syringe 900, in its interior. The medicament, e.g. a liquid medicament or drug Dr, may be arranged in the pre-filled syringe 900. It should be noted that the following use of the term pre-filled syringe 900 does not limit the design of the container to a pre-filled syringe. Rather, containers other than a prefilled syringe may also be considered for implementation. The device body 700 may be provided to retain and/or may retain a needle 908, see FIG. 1C. In other words, the needle 908 may be arranged or may be arranged in the device body 700. The needle 908 can be an integral part of the pre-filled syringe 900 or container, e.g. permanently or releasable connected to a body of the medicament container, or separate from the medicament container. In the first case, the medicament container may be a syringe. In the second case, the medicament container may be a cartridge. In case a cartridge is used as medicament container, initially, the medicament container and the needle can be fluidly disconnected and fluid communication between the medicament container interior and the needle 908 may only be established during operation of the drug delivery device 100. An optional medicament container carrier, such as a syringe holder 800, may be used to support and/or carry the medicament container within the device body 700.

A drive mechanism 101 provided to drive a drug delivery operation may expediently be provided in the device body 700. The drive mechanism 101 may comprise a plunger 1000. The drug delivery device 100 may further comprise a drive energy source, e.g. a drive spring 1100, such as a compression spring, (not explicitly shown). The drive energy source may be arranged to drive the plunger 1000 in a distal direction D relative to the medicament container during the drug delivery operation. During this movement, a plunger stopper 910, which may be movably retained in the medicament container, i.e. the pre-filled syringe 900, and may seal the medicament container, may be displaced towards an outlet of the medicament container medicament to dispense the drug Dr or medicament retained within the medicament container through the outlet. The outlet may be formed or defined by the needle 908, see FIG. 1C. Other potential drive energy sources different from the drive spring 1100 comprise an electrical power cell or battery for driving the plunger 1000 by a motor or a reservoir suitable to provide gas pressure, where the gas pressure can be used to drive the drug delivery operation.

The drug delivery device 100 may be an autoinjector. The energy for driving the drug delivery operation in an autoinjector may be provided by components integral to drug delivery device 100 and does not have to be loaded into the device by the user during the operation of device 100 as is the case in many spring-driven pen-type variable dose injectors, where, usually, the energy is loaded into the spring by the user during a dose setting procedure.

The drug delivery device 100 may expediently be a single shot device, i.e. it is provided to dispense only one dose. The drug delivery device 100 may be a disposable drug delivery device 100, that is to say a device 100 which is disposed of after its use. The device 100 may be a pen-type device. The pre-filled syringe 900 and/or the needle 908 may be axially secured within the drug delivery device 100, e.g. within the device body 700, or may be movable relative to the device body 700, e.g. for piercing the skin. In the first case, the user may have to perform the movement for piercing the skin with needle 908. In the second case, piercing of the skin by the needle 908 may be driven by a needle insertion mechanism of the drug delivery device 100. Automatic needle retraction may be used as well.

As depicted in FIG. 1A, the drug delivery device 100 may further comprises a cap 200. The cap 200 may be arranged at a distal end DE of the drug delivery device 100. The cap 200 may be detachably connected to the remainder of device 100, e.g. the device body 700 and/or another component or member of the drug delivery device 100. The cap 200 may cover the distal end DE of the remainder of the drug delivery device 100 and/or a needle passage opening through which needle 908, e.g. a distal needle tip, may protrude to pierce the skin for the drug delivery operation. The cap 200 may comprise a needle shield remover, such as a grabber 400, which may engage a rigid needle shield (RNS) 914, which may cover the needle 908 such that the RNS 914 is removed from the needle 908 together with the cap 200, e.g. when the cap 200 is detached or disconnected from device 100.

The device body 700 may expediently cover the majority of the length of drug delivery device 100, e.g. 60 percent or 70 percent or more of the entire length of the drug delivery device 100 (with the cap 200 attached and/or with the cap 200 detached).

FIG. 1B illustrates the drug delivery device 100 with the cap 200 being removed. According to FIG. 1B, the device 100 may be in a state ready to be operated, e.g. ready to perform a drug delivery operation when the operation is triggered. As depicted, the drug delivery device 100 may further comprise a needle cover 500. The needle cover 500 may protrude distally from the device body 700 and/or may have been covered by the cap 200 when the cap 200 was still attached to the device body 700. The needle cover 500 may be movable relative to the device body 700 from an initial position or first position to a second position or trigger position. The needle cover 500 may be provided to extend beyond the distal tip of needle 908 which may protrude from the device body 700 before the drug delivery operation is commenced. The needle cover 500 may be movable in the proximal direction P relative to housing 102. During this movement, e.g. before the needle cover 500 reaches the second position, the needle 908 may pierce the skin of the user.

The needle cover 500 may serve as a trigger member of the drug delivery device 100. The needle cover 500 as a trigger member, when displaced proximally from the initial or first position depicted in FIG. 1B to the second or trigger position (see FIG. 1C), may automatically initialize the drug delivery operation, for example, when it is in the second position. The drug delivery operation can be initialized by removing a mechanical lock which prevents movement of plunger 1000 in the distal direction D or by moving the plunger 1000 to disengage a mechanical lock via moving the needle cover 500. Alternatively, the needle cover 500 when moved from the first position to the second position and expediently when in the second position may only enable triggering of the drug delivery operation. In this case, a separate trigger member, e.g. a trigger button on the proximal end PE of the device body 700, may be provided to initiate the drug delivery operation. Operating the trigger button to initiate the drug delivery operation may only be possible when needle cover 500 is in the second position. In yet another alternative, the needle cover 500 may only be provided to prevent needle stick injuries before and/or after use of drug delivery device 100. In this case, the needle cover 500 may be completely decoupled from the drive mechanism 101 and/or not may be involved in triggering or enabling triggering of the drug delivery operation at all.

The needle cover 500 may be provided to bear against the skin of a user during injection. Hence, the distal surface of the needle cover 500 may provide a bearing surface or a skin contact surface 501. The skin contact surface 501 may delimit and/or extend around a needle passage opening provided in needle cover 500. The skin contact surface 501 may be ring-like, oval, elliptic, rectangular, quadratic, etc., circumferentially closed and/or be defined by an inward protrusion protruding radially from an inner wall of needle cover 500, e.g. a distal cylindrical portion thereof. The skin contact surface 501 may be expediently the distal end surface of needle cover 500, e.g. facing distally. The syringe with the needle may be axially fixed in the device. Needle insertion into the skin is expediently done manually and not by displacing the syringe relative to the device body 700.

FIG. 1C illustrates needle cover 500 in the second position relative to device body. This is the position when the drug delivery operation has been initiated, can be initiated, and/or when the needle 908 pierces the skin, for example. The needle 908 may protrude axially from the skin contact surface 501 of the drug delivery device 100 (e.g., through the needle passage opening in needle cover 500) and, by the distance with which it protrudes over the skin contact surface 501, penetrate the skin (the skin is not shown in this representation). This distance may be characteristic for or be equal to the injection depth. The device 100 may be maintained in contact with the skin until the drug delivery operation of drug Dr has been completed, which may be indicated by an optional audible, tactile, and/or visual indication or feedback provided by the drug delivery device 100.

After the drug delivery operation has been completed, e.g. the plunger 1000 has moved distally, the device 100 may be removed from the skin (see FIG. 1D). The needle cover 500 may be biased relative to the device body 700 towards the first position by a needle cover spring 600 (not shown). Thus, when the device 100 is removed from the skin, the needle cover 500 may be moved towards the first position with respect to the device body 700. The needle cover 500 may be moved distally, e.g. beyond its first position, into a final, third or locked position relative to the device body 700. In this position the needle cover 500 may expediently be axially locked relative to the device body 700 against movement in the proximal direction P, e.g. by a locking engagement between a locking feature of needle cover 500 and the device body 700. As it is axially locked, the needle cover 500 may no longer be displaced proximally relative to device body 700 into the second position and/or into the first position. This may protect the user from needle stick injuries after use. In this state, device 100 may be locked, see FIG. 1D. The needle cover may protrude further from the device body in the third cover position Z than in the first cover position X.

### List of Drugs

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20 °C), or refrigerated temperatures (e.g., from about-4 °C to about 4 °C). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. For example, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly (A21), Arg (B31), Arg (B32) human insulin (insulin glargine); Lys (B3), Glu (B29) human insulin (insulin glulisine); Lys (B28), Pro (B29) human insulin (insulin lispro); Asp (B28) human insulin (insulin aspart); human insulin, wherein proline in position B28 is replaced by Asp, Lys, Leu, Val or Ala and wherein in position B29 Lys may be replaced by Pro; Ala (B26) human insulin; Des (B28-B30) human insulin; Des (B27) human insulin and Des (B30) human insulin.

Examples of insulin derivatives are, for example, B29-N-myristoyl-des (B30) human insulin, Lys (B29) (N-tetradecanoyl)-des (B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N−(N-palmitoyl-gamma-glutamyl)-des (B30) human insulin, B29-N-omega-carboxypentadecanoyl-gamma-L-glutamyl-des (B30) human insulin (insulin degludec, Tresiba®); B29-N−(N-lithocholyl-gamma-glutamyl)-des (B30) human insulin; B29-N-(ω-carboxyheptadecanoyl)-des (B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F (ab) and F(ab′) 2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Further examples of APIs for the prophylaxis of hemophilia A or B, with or without inhibitors, include an siRNA targeting antithrombin. An example of an siRNA targeting antithrombin is fitusiran. The term “prophylaxis” and “prophylactic treatment” are used interchangeably herein.

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1: 2014 (E). As described in ISO 11608-1: 2014 (E), needle-based injection systems may be broadly distinguished into multi-dose container systems and single-dose (with partial or full evacuation) container systems. The container may be a replaceable container or an integrated non-replaceable container.

As further described in ISO 11608-1: 2014 (E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1: 2014 (E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1: 2014 (E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

Fitusiran as the API for the medicament in the device

Fitusiran is a synthetic, chemically modified double-stranded small interfering RNA (siRNA) oligonucleotide covalently linked to a tri-antennary N-acetyl-galactosamine (GalNAc) ligand targeting AT3 mRNA in the liver, thereby suppressing the synthesis of antithrombin. See, e.g., Pasi et al., N Engl J Med. (2017) 377 (9): 819-28. The nucleosides in each strand of fitusiran are connected through either 3′-5′ phosphodiester or phosphorothioate linkages, thus forming the sugar-phosphate backbone of the oligonucleotide.

The sense strand and the antisense strand contain 21 and 23 nucleotides, respectively. The 3′ end of the sense strand is conjugated to the GalNAc containing moiety (referred to herein as L96) through a phosphodiester linkage. The sense strand contains two consecutive phosphorothioate linkages at its 5′ end. The antisense strand contains four phosphorothioate linkages, two at the 3′ end and two at the 5′ end. The 21 nucleotides of the sense strand hybridize with the complementary 21 nucleotides of the antisense strand, thus forming 21 nucleotide base pairs and a two-base overhang at the 3′-end of the antisense strand. See also U.S. Pat. Nos. 9,127,274, 11,091,759, US2020/0163987A1, and WO 2019/014187, the entire contents each of which are expressly incorporated herein by reference.

The two nucleotide strands of fitusiran are shown below:

sense strand:
(SEQ ID NO: 1)
5′Gf-ps-Gm-ps-Uf-Um-Af-Am-Cf-Am-Cf-Cf-Af-Um-
Uf-Um-Af-Cm-Uf-Um-Cf-Am-Af-L96 3′,
and
antisense strand:
(SEQ ID NO: 2)
5′Um-ps-Uf-ps-Gm-Af-Am-Gf-Um-Af-Am-Af-Um-Gm-
Gm-Uf-Gm-Uf-Um-Af-Am-Cf-Cm-ps-Am-ps-Gm 3′,

wherein
   Af=2′-deoxy-2′-fluoroadenosine
   Cf=2′-deoxy-2′-fluorocytidine
   Gf=2′-deoxy-2′-fluoroguanosine
   Uf=2′-deoxy-2′-fluorouridine
   Am=2′-O-methyladenosine
   Cm=2′-O-methylcytidine
   Gm=2′-O-methylguanosine
   Um=2′-O-methyluridine
   “-” (hyphen)=3′-5′ phosphodiester linkage sodium salt
   “-ps-”=3′-5′ phosphorothioate linkage sodium salt
      and wherein L96 has the following formula:

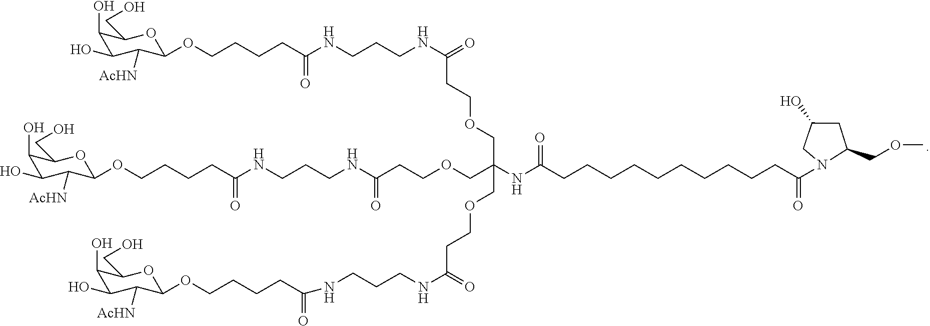 [SMILES: COC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

As used herein, the terms 2′-deoxy-2′-fluoroadenosine and 2′-fluoroadenosine may be used interchangeably.

As used herein, the terms 2′-deoxy-2′-fluorocytidine and 2′-fluorocytidine may be used interchangeably.

As used herein, the terms 2′-deoxy-2′-fluoroguanosine and 2′-fluoroguanosine may be used interchangeably.

As used herein, the terms 2′-deoxy-2′-fluorouridine and 2′-fluorouridine may be used interchangeably.

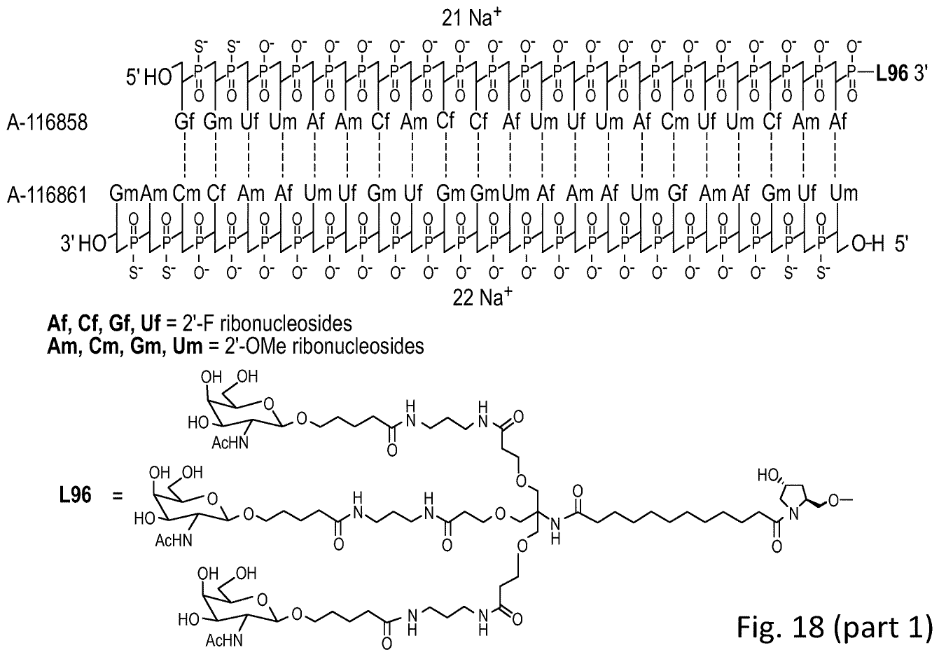

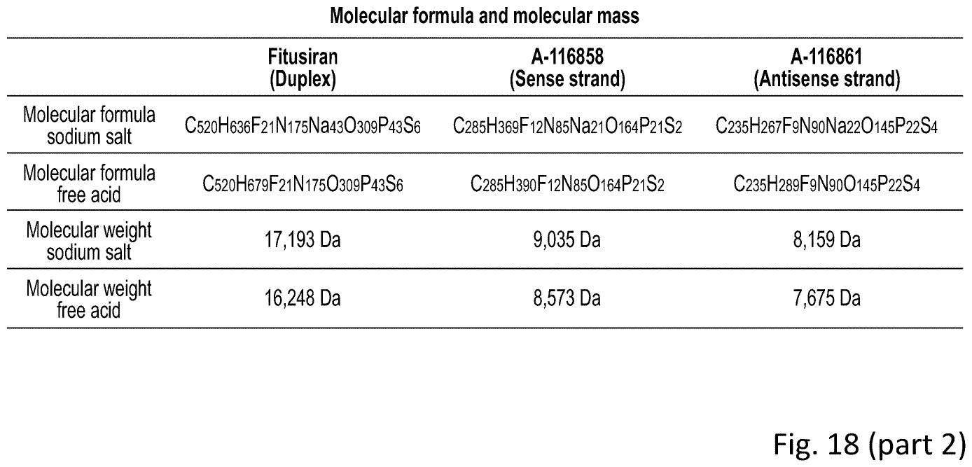

The expanded structural formula, molecular formula, and molecular weight of fitusiran, e.g. sodium form, are shown in FIG. 18.

The structure of fitusiran can also be described using the following diagram, wherein the X is O:

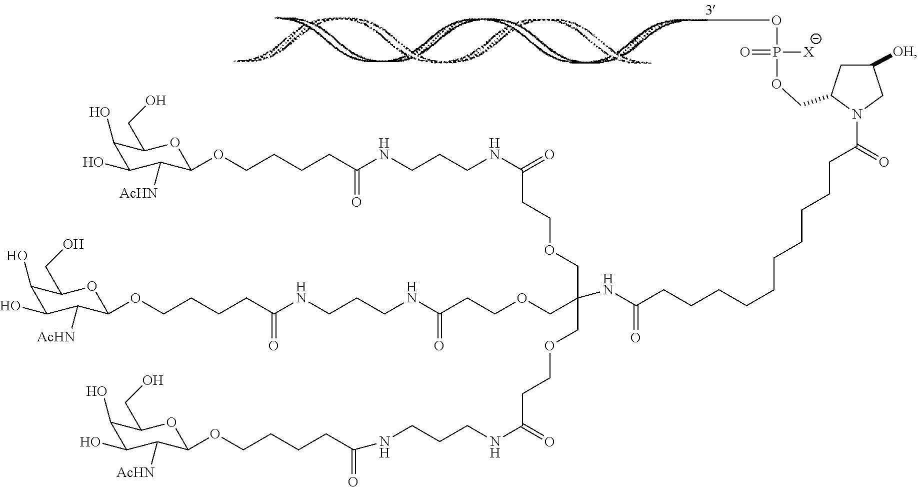

Fitusiran is shown in FIG. 18 in sodium salt form.

In some embodiments, the device delivers fitusiran in an aqueous solution, wherein fitusiran is at a concentration of about 40 to about 200 mg/ml (e.g., about 50 to about 150 mg/mL, about 80 to about 110 mg/mL, or about 90 to about 110 mg/mL). As used herein, values intermediate to recited ranges and values are also intended to be part of this disclosure. In addition, ranges of values using a combination of any of recited values as upper and/or lower limits are intended to be included. In further embodiments, the pharmaceutical formulation comprises fitusiran in an aqueous solution at a concentration of about 40, about 50, about 75, about 100, about 125, about 150, or about 200 mg/mL. In certain embodiments, fitusiran is provided in an aqueous solution at a concentration of about 100 mg/mL.

The term “deliver,” “delivers,” or “delivering” is intended to mean “administer,” “administers,” or “administering.”

Unless specifically stated or otherwise evident from the context, as used herein, the term “approximately” or “about” refers to a value that is within an acceptable error range for a particular value determined by a person of ordinary skill, a portion of which will depend on how the measurement or determination is made. For example, “approximately” or “about” may mean a range of up to 10% (i.e., ±10%). Therefore, “approximately” or “about” can be understood as greater than or less than 10%, 9%, 8%, 7%, 6%, 5%, 4%, 3%, 2%, 1%, 0.5%, 0.1%, 0.05%, 0.01%, or 0.001%. When a specific value is provided in this disclosure, unless otherwise stated, the meaning of “approximately” or “about” should be assumed to be within an acceptable error range for that specific value.

While the fitusiran dosage weight described herein refers to the weight of fitusiran free acid (active moiety), administration of fitusiran to patients herein refers to administration of fitusiran sodium (drug substance) provided in a pharmaceutically suitable aqueous solution (e.g., a phosphate-buffered saline at a physiological pH). For example, about 100 mg/mL fitusiran means about 100 mg of fitusiran free acid (equivalent to about 106 mg fitusiran sodium, the drug substance) per mL. Unless otherwise indicated, a fitusiran weight recited in the present disclosure is the weight of fitusiran free acid (the active moiety).

In some embodiments, a pharmaceutical formulation in the device comprises fitusiran in a phosphate-buffered saline. The phosphate concentration in the solution may be about 1 to about 10 mM (e.g., about 2, about 3, about 4, about 5, about 6, about 7, about 8, or about 9 mM), with a pH of about 6.0-8.0. The pharmaceutical formulations herein may include a stabilizing agent such as EDTA. The pharmaceutical formulations may be preservative-free. In some embodiments, the fitusiran pharmaceutical formulation in the device is preservative-free and comprises, consists of, or consists essentially of about 100 mg of fitusiran per mL of an approximately 5 mM phosphate buffered saline (PBS) solution. In some embodiments, the fitusiran pharmaceutical formulation in the device is preservative-free and comprises, consists of, or consists essentially of fitusiran in an approximately 5 mM phosphate buffered saline (PBS) solution. The PBS solution is composed of sodium chloride, dibasic sodium phosphate (heptahydrate), and monobasic sodium phosphate (monohydrate). Sodium hydroxide solution and diluted phosphoric acid may be used to adjust the pH of the formulation to about 7.0 or about 7.1.

In some embodiments, the fitusiran formulation in the device for subcutaneous delivery contains fitusiran in a 5 mM phosphate buffered saline having 0.64 mM NaH₂PO₄, 4.36 mM Na₂HPO₄, and 84 mM NaCl at pH 7.0. In certain embodiments, the formulation of fitusiran solution for subcutaneous delivery is shown in Table 1 below:

TABLE 1
Exemplary Fitusiran Formulation

Formulation

| | Percentage | Per ml |
|---|---|---|
| Components | [%] | [mg] |
| Fitusiran (active moiety) | 10 | 100 [106] |
| [equivalent to fitusiran sodium] | | |
| Sodium chloride | 0.49 | 4.909 |
| Dibasic sodium phosphate | 0.12 | 1.169 |
| (heptahydrate) | | |
| Monobasic sodium phosphate | <0.01 | 0.0885 |
| (monohydrate) | | |
| Phosphoric acid, concentrated | — | q.s. pH 7.0 |
| Sodium hydroxide | — | q.s. pH 7.0 |
| Water for subcutaneous | q.s. 100 | q.s. 1 mL |
| delivery | | |

*q.s.: quantum satis

In some embodiments, the formulation of fitusiran solution for subcutaneous delivery with the device can be described as shown in Table 2 below.

TABLE 2
Exemplary Fitusiran Formulation

| Components | Formulation (mg) |
|---|---|
| Fitusiran (active moiety) | 100 [106] |
| [equivalent to fitusiran sodium] | |
| NaH₂PO₄*H₂O | 0.0885 |
| Na₂HPO₄*7H₂O | 1.169 |
| NaCl | 4.909 |
| 0.1N NaOH | q.s. |
| 0.1M H₃PO₄ | q.s. |
| Purified water | Ad 1 mL |

In some embodiments, the device may be used to deliver a single dose of fitusiran wherein the single dose comprises about 20 to about 80 mg of fitusiran (e.g., about 20 mg, about 25 mg, about 30 mg, about 40 mg, about 50 mg, or about 80 mg). In some embodiments, the device may be used to deliver single dose of fitusiran, wherein the single dose comprises about 1 to about 30 mg of fitusiran (e.g., about 1.25 mg, about 2.5 mg, about 5 mg, about 10 mg, about 20 mg, or about 30 mg).

In one embodiment, the device may be used to deliver a single dose of about 80 mg of fitusiran.

In one embodiment, the device may be used to deliver a single dose of about 50 mg of fitusiran.

In one embodiment, the device may be used to deliver a single dose of about 20 mg of fitusiran.

In one embodiment, the device may be used to deliver a single dose of about 30 mg of fitusiran.

In one embodiment, the device may be used to deliver a single dose of about 10 mg of fitusiran.

In one embodiment, the device may be used to deliver a single dose of about 5 mg of fitusiran.

In one embodiment, the device may be used to deliver a single dose of about 2.5 mg of fitusiran. In one embodiment, the device may be used to deliver a single dose of about 1.25 mg of fitusiran.

In some embodiments, the single dose of fitusiran may be delivered in about 0.5 mL to about 1 mL delivery volumes (e.g., about 0.5 mL, about 0.6 mL, about 0.7 mL, about 0.8 mL, about 0.9 mL, or about 1 mL). Other delivery volumes described herein may also be used.

In one embodiment, the device may be used to deliver a single dose of about 80 mg of fitusiran in about 0.8 mL (about 100 mg fitusiran/mL). In one embodiment, the device may be used to deliver a single dose of about 50 mg of fitusiran in about 0.5 mL (about 100 mg fitusiran/mL). In one embodiment, the device may be used to deliver a single dose of about 20 mg of fitusiran in about 0.5 mL (about 40 mg fitusiran/mL). In one embodiment, the device may be used to deliver a single dose of about 30 mg of fitusiran in about 0.5 mL (about 60 mg fitusiran/mL). In one embodiment, the device may be used to deliver a single dose of about 10 mg of fitusiran in about 0.5 mL (about 20 mg fitusiran/mL). In one embodiment, the device may be used to deliver a single dose of about 5 mg of fitusiran in about 0.5 mL (about 10 mg fitusiran/mL). In one embodiment, the device may be used to deliver a single dose of about 2.5 mg of fitusiran in about 0.5 mL (about 5 mg fitusiran/mL). In one embodiment, the device may be used to deliver a single dose of about 1.25 mg of fitusiran in about 0.5 mL (about 2.5 mg fitusiran/mL).

In one embodiment, the device delivers fitusiran at a prophylactically effective amount to prophylactically treat hemophilia (e.g., hemophilia A or B, in a patient with or without inhibitors) in a patient in need thereof (e.g., a hemophilia A or B patient, with or without inhibitors). “Prophylactically effective amount” refers to the amount of fitusiran that helps the patient with hemophilia A or B, with or without inhibitors to achieve a desired clinical endpoint such as reducing the Annualized Bleeding Rate (ABR), Annualized Joint Bleeding Rate (AjBR),

Annualized Spontaneous Bleeding Rate (AsBR), or the frequency of bleeding episodes. As used herein in the context of fitusiran, the term “treat” “treating,” or “treatment” includes prophylactic treatment of the disease and refers to achievement of a desired clinical endpoint.

A hemophilia A or B patient with inhibitors refers to a patient who has developed alloantibodies to the factor he/she has previously received (e.g., factor VIII for hemophilia A patients or factor IX for hemophilia B patients). A hemophilia A or B patient with inhibitors may become refractory to replacement coagulation factor therapies. A patient without inhibitors refers to a patient who does not have such alloantibodies. The present treatment methods may be beneficial for hemophilia A patients with inhibitors, as well as for hemophilia B patients with inhibitors.

As used herein, a patient with “hemophilia A or B, with or without inhibitors,” or refers to 1) a hemophilia A patient with inhibitors, or 2) a hemophilia B patient with inhibitors, 3) a hemophilia A patient without inhibitors, or 4) a hemophilia B patient without inhibitors. As used herein, a patient refers to a human patient. A patient can also refer to a human subject.

In some embodiments, the device may be used to prophylactically treat a patient with hemophilia A or B, with or without inhibitors, with a subcutaneous dose of about 50 mg of fitusiran once every two months (or every eight weeks). In other embodiments, the device may be used to prophylactically treat a patient with hemophilia A or B, with or without inhibitors, with a subcutaneous dose of about 50 mg of fitusiran every month (or every four weeks). In yet other embodiments, the device may be used to prophylactically treat a patient with hemophilia A or B, with or without inhibitors, with a subcutaneous dose of about 80 mg of fitusiran every two months (or every eight weeks). In yet other embodiments, the device may be used to prophylactically treat a patient with hemophilia A or B, with or without inhibitors, with a subcutaneous dose of about 80 mg of fitusiran every month (or every four weeks). In yet other embodiments, the device may be used to prophylactically treat a patient with hemophilia A or B, with or without inhibitors, with a subcutaneous dose of about 20 mg of fitusiran every two months (or every eight weeks). In yet other embodiments, the device may be used to prophylactically treat a patient with hemophilia A or B, with or without inhibitors, with a subcutaneous dose of about 20 mg of fitusiran every month (or every four weeks). In yet other embodiments, the device may be used to prophylactically treat a patient with hemophilia A or B, with or without inhibitors, with a subcutaneous dose of about 10 mg of fitusiran every month (or every four weeks). In yet other embodiments, the device may be used to prophylactically treat a patient with hemophilia A or B, with or without inhibitors, with a subcutaneous dose of fitusiran at about 30 mg every month (or every four weeks). In yet other embodiments, the device may be used to prophylactically treat a patient with hemophilia A or B, with or without inhibitors, with a subcutaneous dose of fitusiran at about 5 mg every month (or every four weeks). In yet other embodiments, the device may be used to prophylactically treat a patient with hemophilia A or B, with or without inhibitors, with a subcutaneous dose of fitusiran at about 2.5 mg every month (or every four weeks). In yet other embodiments, the device may be used to prophylactically treat a patient with hemophilia A or B, with or without inhibitors, with a subcutaneous dose of fitusiran at about 1.25 mg every month (or every four weeks).

Accordingly, provided herein is a method of prophylactic treatment of a patient with hemophilia A or hemophilia B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of prophylactic treatment of a patient with hemophilia A or hemophilia B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Further provided herein is a method of reducing the frequency of bleeding episodes in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the frequency of bleeding episodes in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Also, provided herein is a method of reducing the ABR in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the ABR in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Also, provided herein is a method of reducing the AjBR in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). The fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the AjBR in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Also, provided herein is a method of reducing the AsBR in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the AsBR in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 ml PBS (at a concentration of about 100 mg fitusiran/mL).

In some embodiments, the device may be used to prophylactically treat a patient with hemophilia A or B, with or without inhibitors, with a subcutaneous dose of about 50 mg of fitusiran about once every two months (or about every eight weeks). In other embodiments, the device may be used to prophylactically treat a patient with hemophilia A or B, with or without inhibitors, with a subcutaneous dose of about 50 mg of fitusiran about every month (or about every four weeks). In yet other embodiments, the device may be used to prophylactically treat a patient with hemophilia A or B, with or without inhibitors, with a subcutaneous dose of about 80 mg of fitusiran about every two months (or about every eight weeks). In yet other embodiments, the device may be used to prophylactically treat a patient with hemophilia A or B, with or without inhibitors, with a subcutaneous dose of about 80 mg of fitusiran about every month (or about every four weeks). In yet other embodiments, the device may be used to prophylactically treat a patient with hemophilia A or B, with or without inhibitors, with a subcutaneous dose of about 20 mg of fitusiran about every two months (or about every eight weeks). In yet other embodiments, the device may be used to prophylactically treat a patient with hemophilia A or B, with or without inhibitors, with a subcutaneous dose of about 20 mg of fitusiran about every month (or every four weeks). In yet other embodiments, the device may be used to prophylactically treat a patient with hemophilia A or B, with or without inhibitors, with a subcutaneous dose of about 10 mg of fitusiran about every month (or about every four weeks). In yet other embodiments, the device may be used to prophylactically treat a patient with hemophilia A or B, with or without inhibitors, with a subcutaneous dose of fitusiran at about 30 mg about every month (or about every four weeks). In yet other embodiments, the device may be used to prophylactically treat a patient with hemophilia A or B, with or without inhibitors, with a subcutaneous dose of fitusiran at about 5 mg about every month (or about every four weeks). In yet other embodiments, the device may be used to prophylactically treat a patient with hemophilia A or B, with or without inhibitors, with a subcutaneous dose of fitusiran at about 2.5 mg about every month (or about every four weeks). In yet other embodiments, the device may be used to prophylactically treat a patient with hemophilia A or B, with or without inhibitors, with a subcutaneous dose of fitusiran at about 1.25 mg about every month (or about every four weeks).

Accordingly, provided herein is a method of prophylactic treatment of a patient with hemophilia A or hemophilia B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered about every month (or about every four weeks) or about once every two months (or about every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of prophylactic treatment of a patient with hemophilia A or hemophilia B, with or without inhibitors, may comprise subcutaneously delivering with the device about 50 mg of fitusiran to the patient in need thereof about every month (or about every four weeks) or about once every two months (or about every eight weeks). The about 50 mg of fitusiran may be delivered in about 0.5 mL PBS (at a concentration of about 100 mg fitusiran/mL).

Further provided herein is a method of reducing the frequency of bleeding episodes in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered about every month (or about every four weeks) or about once every two months (or about every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the frequency of bleeding episodes in a patient with hemophilia A or B, with or without inhibitors, may comprise subcutaneously delivering with the device about 50 mg of fitusiran to the patient in need thereof about every month (or about every four weeks) or about once every two months (or about every eight weeks). The about 50 mg of fitusiran may be delivered in about 0.5 mL PBS (at a concentration of about 100 mg fitusiran/mL).

Also, provided herein is a method of reducing the ABR in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered about every month (or about every four weeks) or about once every two months (or about every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the ABR in a patient with hemophilia A or B, with or without inhibitors, may comprise subcutaneously delivering with the device about 50 mg of fitusiran to the patient in need thereof about every month (or about every four weeks) or about once every two months (or about every eight weeks). The about 50 mg of fitusiran may be delivered in about 0.5 mL PBS (at a concentration of about 100 mg fitusiran/mL).

Also, provided herein is a method of reducing the AjBR in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered about every month (or about every four weeks) or about once every two months (or about every eight weeks). The fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the AjBR in a patient with hemophilia A or B, with or without inhibitors, may comprise subcutaneously delivering with the device about 50 mg of fitusiran to the patient in need thereof about every month (or about every four weeks) or about once every two months (or about every eight weeks). The about 50 mg of fitusiran may be delivered in about 0.5 mL PBS (at a concentration of about 100 mg fitusiran/mL).

Also, provided herein is a method of reducing the AsBR in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered about every month (or about every four weeks) or about once every two months (or about every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the AsBR in a patient with hemophilia A or B, with or without inhibitors, may comprise subcutaneously delivering with the device about 50 mg of fitusiran to the patient in need thereof about every month (or about every four weeks) or about once every two months (or about every eight weeks). The about 50 mg of fitusiran may be delivered in about 0.5 mL PBS (at a concentration of about 100 mg fitusiran/mL).

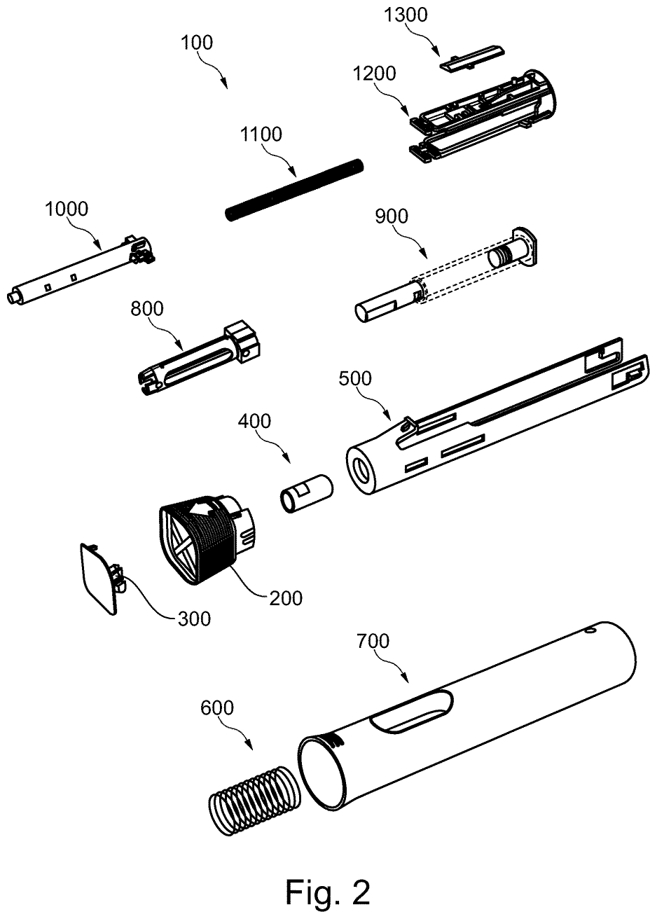

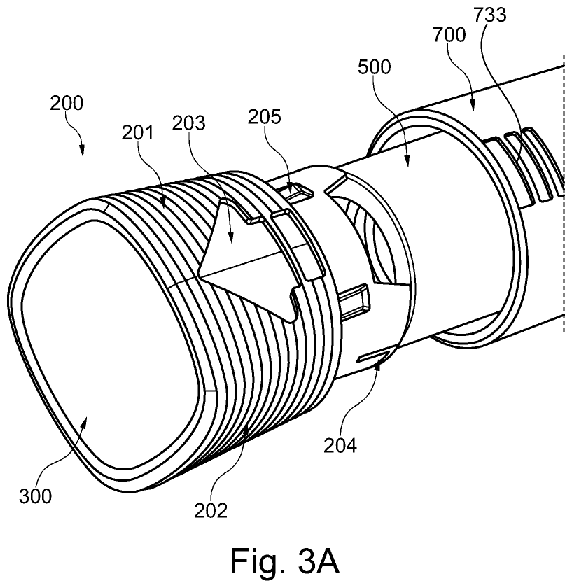

2. Example of Drug Delivery Device without or with Separate Syringe Holder (FIGS. 1A to 1D and FIG. 2)

FIG. 2 illustrates an exploded view of an example of the drug delivery device 100 without or with an optional separate syringe holder 800. The drug delivery device 100 may be an auto-injector suitable for automatic injection of the drug Dr. The triggering of the injection process may be done manually, i.e. by the user.

The drug delivery device 100 may comprise:
The removable cap 200 and a cap lid 300. The cap 200 with the cap lid 300 may be prevented from reattaching after use of the drug delivery device 100. Details of the cap 200 and the cap lid 300 are explained below in section 3.
The grabber 400 that is mounted on the cap 200 and that is configured to remove the RNS 914 or a soft needle shield SNS 914 of the pre-filled syringe 900. Details of the grabber 400 are explained below in section 4.
The needle cover 500 that may be arranged telescoped within the device body 700. Details of the needle cover 500 are explained below in section 5.
The needle cover spring 600 that biases the needle cover 500 in the distal direction D. Details of the needle cover spring 600 are explained below in section 6.
The device body 700 that is essentially cylindrically and that may comprise a distal opening configured to insert the needle cover 500 and a proximal opening configured to insert a drive spring holder 1200 that may have the function of a rear case. Details of the device body 700 are explained below in section 7.
The optional syringe holder 800. Mounting of the pre-filled syringe 900 without a syringe holder is described below in section 7 in more detail. Details of the syringe holder 800 are explained below in section 8.
The pre-filled syringe 900. Details of the pre-filled syringe 900 are explained below in section

9. Alternatively, a cartridge configured to be connected to a removable needle or any other drug container may be used.
The plunger 1000. Details of the plunger 1000 are explained below in section 10. The plunger 1000 may be used to expel the drug Dr out of pre-filled syringe 900.
The drive spring 1100. Details of the drive spring 1100 are explained below in section 11. The drive spring 1100 may supply the mechanical energy for automatic drug injection. Alternatively, other drive sources may be used, e.g. pneumatic energy or electric energy.
The drive spring holder 1200. Details of the drive spring holder 1200 are explained below in section 12. The drive spring holder 1200 may be a part of the housing, case or device body 700, especially a rear part. The drive spring holder 1200 may be configured to hold the drive spring 1100 and to fulfill other functions, e.g. to support a syringe flange 912 of the pre-filled syringe 900 via two support arms that extend distally from a proximal end plate of the drive spring holder 1200.
A clicker 1300. Details of the clicker 1300 are explained below in section 13. The clicker may be an audible and/or tactile indicator or provide audible and/or tactile feedback, e.g. indicating end of dose delivery or other events.

A control sub-assembly (or front subassembly) may comprise the needle cover 500, the needle cover spring 600 and the device body 700. Control sub-assembly may perform the control of the pre-filled syringe 900.

The plunger 1000, the drive spring 1100, the drive spring holder 1200 and the optional audible indicator or clicker 1300 may be comprised in a drive sub-assembly (or rear sub-assembly).

The drug delivery device 100 may comprise a case designed as a multi-part case. For example, the case may comprise the device body 700 forming a front case and a rear case formed e.g. by the drive spring holder 1200. A portion of the drive spring holder 1200, may be surrounded by the front case or the device body along a longitudinal direction and adapted to close an open proximal end of the front case. A proximal portion of the drive spring holder can protrude from a proximal end of the device body. The case may be adapted to hold the pre-filled syringe 900 as well as the other parts of auto-injector 100.

The pre-filled syringe 900 has the needle 908 arranged at a distal end, e.g. staked to a neck of a syringe body. The pre-filled syringe 900 may be pre-assembled. Typically, a protective needle shield may be removable coupled to the needle 908 of the pre-filled syringe 900. The protective needle shield may be a soft needle shield (e.g. a rubber needle shield SNS) 914 or the RNS 914 which may be composed of an inner rubber material and a full or partial plastic shell. The plunger stopper 910 may be arranged for sealing the pre-filled syringe 900 proximally and for displacing the drug Dr or medicament M contained in the pre-filled syringe 900 through the needle 908. In other exemplary embodiments, instead of the pre-filled syringe 900 a cartridge or a container may be used which includes the drug Dr or medicament M and engages a removable needle (e.g., by threads, snaps, friction, Luer lock, etc.).

In an exemplary embodiment, the cap 200 may be removably disposed at the distal end DE of the device body 700 or the case. The cap 200 may include a grip element the grabber 400 (e.g. comprising barb(s), hook(s), narrowed section(s), etc.) arranged to engage the protective needle shield RNS or SNS 914 of the pre-filled syringe 900. The cap 200 may also engage with the needle cover 500 and/or with the device body 700. The cap 200 may comprise grip features for facilitating removal of the cap 200 (e.g., by twisting and/or pulling the cap 200 relative to the device body 700). Moreover, the cap 200 may comprise a visual and/or tactile indication of the direction for removing the cap 200 from the device body 700, e.g. an arrow. The cap 200 may be a single part integrally formed, e.g. by injection molding. Alternatively, the cap 200 may comprise several parts, e.g. a cap body 201 and a cap lid 300.

In an exemplary embodiment, the needle cover spring 600 may be arranged to bias the needle cover 500 in a distal direction D against the device body 700.

In an exemplary embodiment, the drive spring 1100 may be arranged within the device body 700, e.g. mounted on the drive spring holder 1200. The plunger 1000 may serve for forwarding a force of the drive spring 1100 to the plunge stopper 910 within the pre-filled syringe 900 or within another drug container.

In an exemplary embodiment, the plunger 1000 may be hollow and the drive spring 1100 may be arranged within the plunger 1000 biasing the plunger 1000 in the distal direction D with respect to the device body 700 and/or the drive spring holder 1200.

In another exemplary embodiment, the plunger 1000 may be solid and the drive spring 1100 may engage a proximal end of the plunger 1000. Likewise, the drive spring 1100 could be wrapped around the outer diameter of the plunger 1000 and/or extend within the pre-filed syringe 900.

In an exemplary embodiment, a plunger release mechanism may be arranged for preventing release of the plunger 1000 prior to retraction of the needle cover 500 relative to the device body 700 and for releasing the plunger 1000 once the needle cover 500 is sufficiently retracted.

In an exemplary embodiment, a pre-use needle cover lock mechanism may be arranged to prevent retraction of the needle cover 500 relative to the device body 700 when the cap 200 is in place, thereby avoiding unintentional activation of the autoinjector, i.e. the drug delivery device 100 (e.g., if dropped, during shipping or packaging, etc.).

Moreover, there may be an after-use needle cover lock mechanism that prevents proximal movement of needle cover 500 after use of drug delivery device 100.

When the cap 200 is attached to the drug delivery device 100, axial movement of the cap 200 in the proximal direction P relative the device body 700 may be limited by the cap 200 abutting the device body 700. When the cap 200 is pulled in the distal direction D relative to the device body 700 the grabber 400 of the cap 200 may grasp the RNS or SNS 914 and may allow removal of the RNS or SNS 914, too.

In the shown embodiment, the cap 200 may comprise a closable opening for inserting a front assembling tool. The cap 200 may be permanently closed at its distal end.

The drug delivery device 100 may comprise at least one clicker 1300 for producing an audible and/or tactile feedback of completion of drug Dr or medicament M delivery. In the context of the disclosure, the clicker 1300 may also be referred to as an audible and/or tactile indicator. The audible and/or tactile indicator 1300 may be formed for example as a monostable or bistable spring, e.g. a leaf spring, and may be held in the drive spring holder 1200 or rear case.

The drive spring holder 1200 or rear case may be adapted to prevent axial movement of the pre-filled syringe 900 after assembling, e.g., during storage, transportation and normal use. In detail, the drive spring holder 1200 may comprise at its front-end resilient arms, e.g. two resilient arms. The resilient arms may be formed as labyrinth arms to damp impact forces. The resilient arms may be mounted on more rigid arms, e.g. two rigid arms, of drive spring holder 1200. The rigid arms may extend from a proximal end plate of drive spring holder 1200 into the distal direction. Both rigid arms may be arranged in parallel to each other or essentially in parallel. Drive spring holder 1200 may comprise a central pin for guiding the drive spring 1100. The central pin and the proximal end plate may be an integral part of drive spring holder 1200 or may be separate parts thereof, e.g. integrated into one single part that is separate with regard to drive spring holder 1200.

In an exemplary embodiment, the drug delivery device 100 may be formed from at least two subassemblies, e.g., a control or front subassembly and a drive or rear subassembly, to allow for flexibility as to the time and location of manufacture of the subassemblies and final assembly with the pre-filled syringe 900.

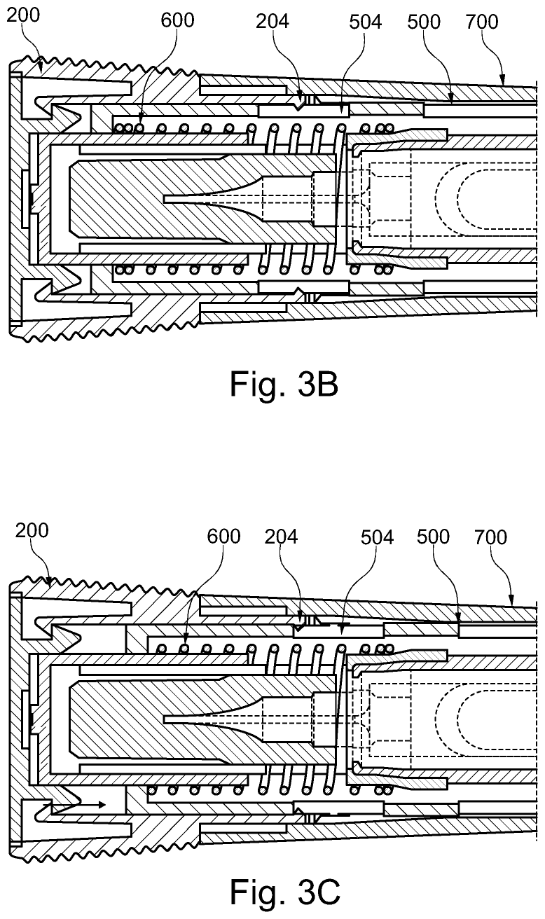

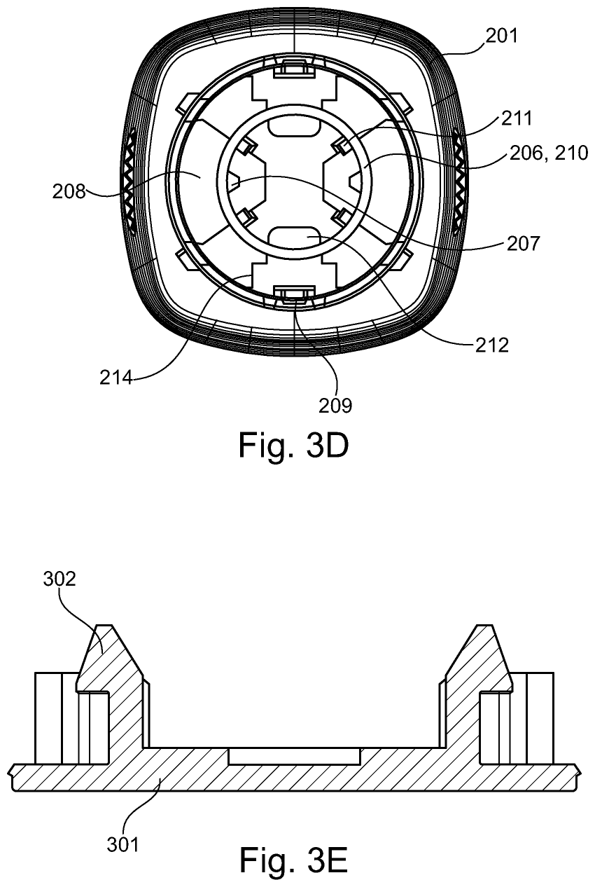

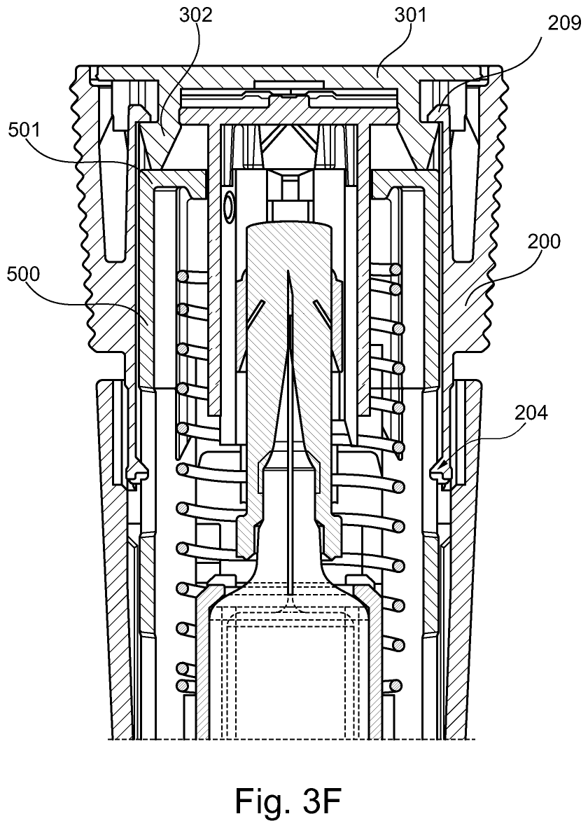

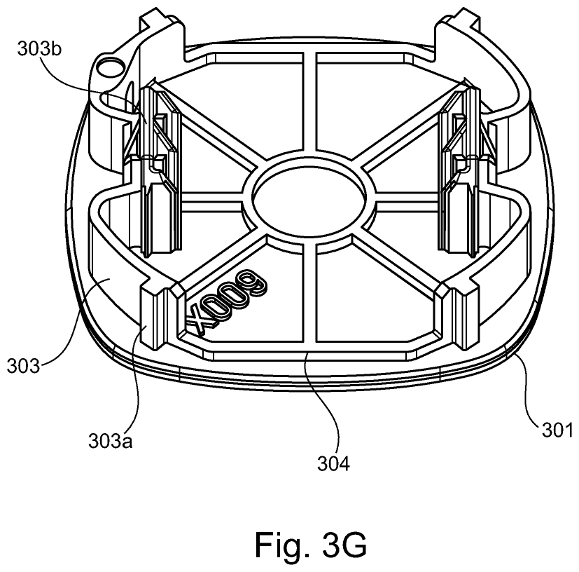

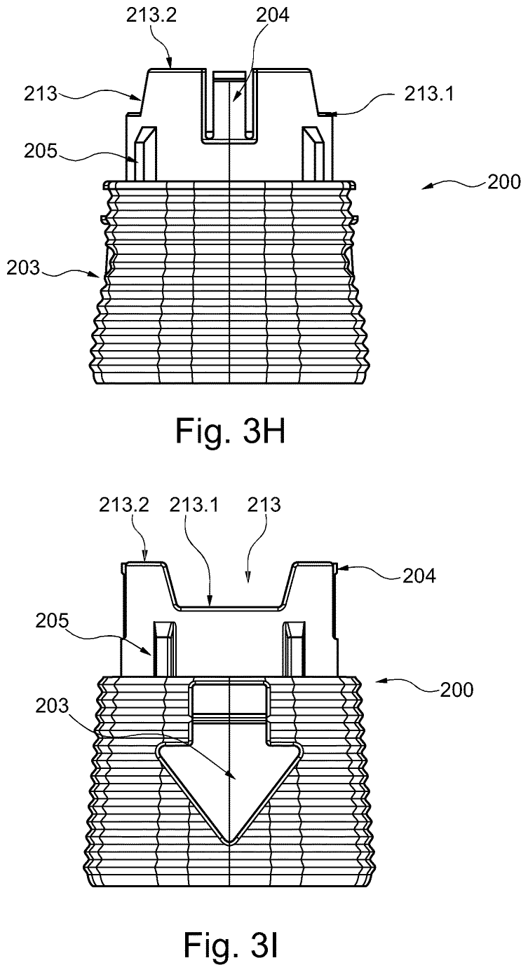

### 3. Cap and Cap Lid (FIGS. 3A to 3I)

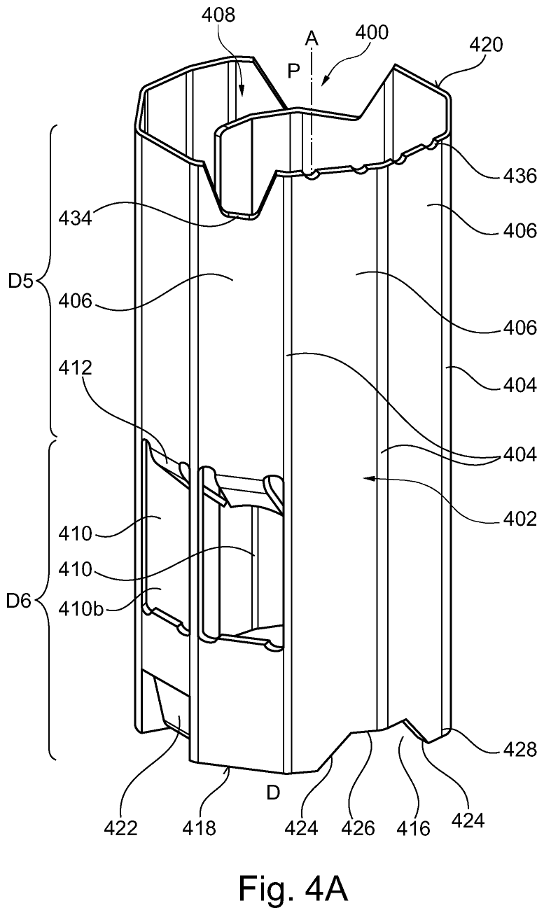

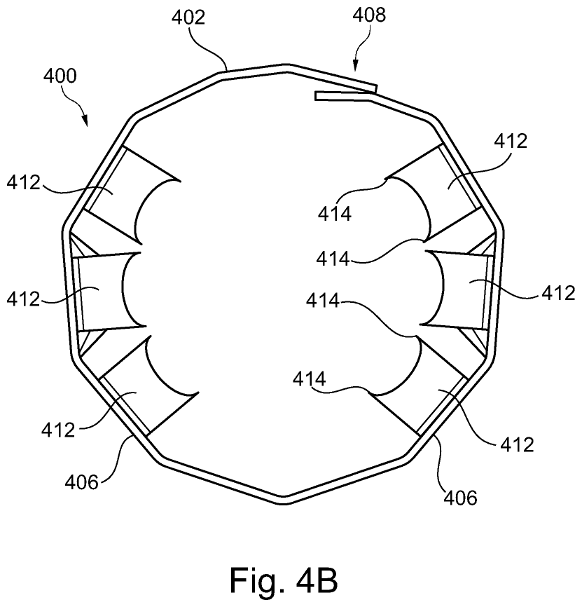

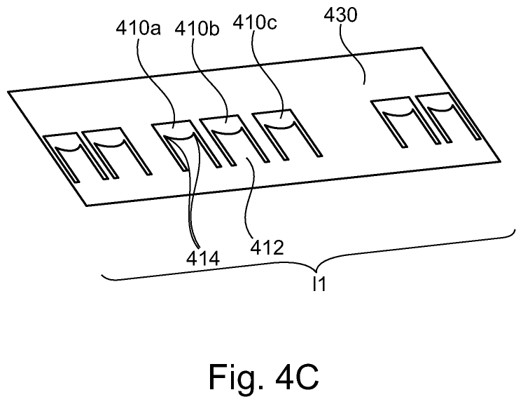

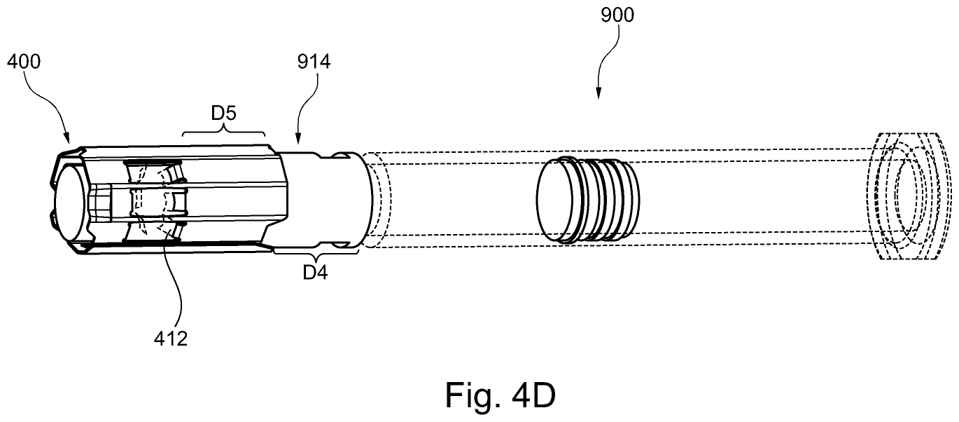

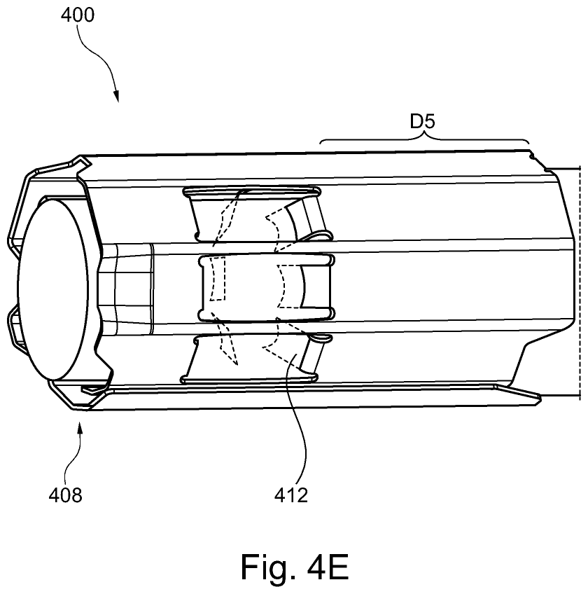

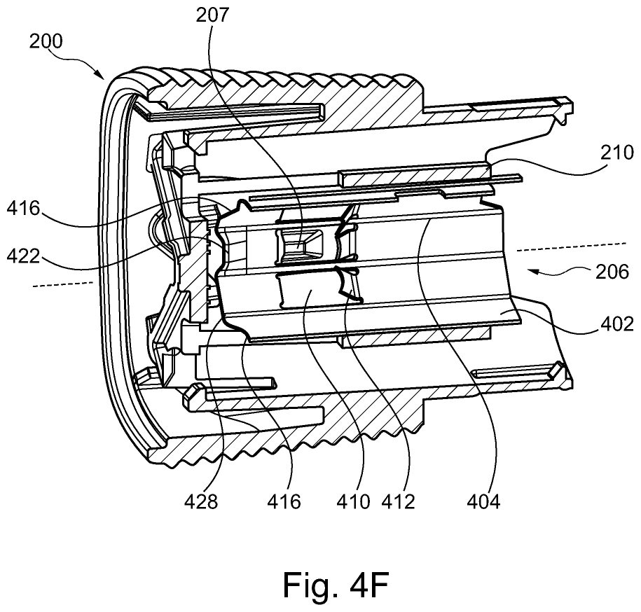

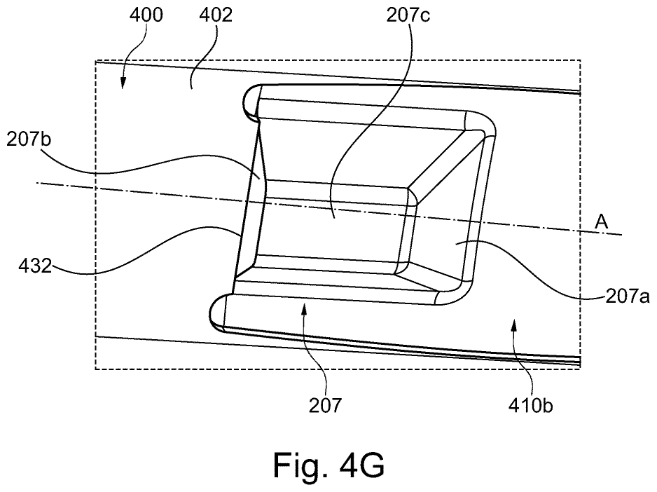

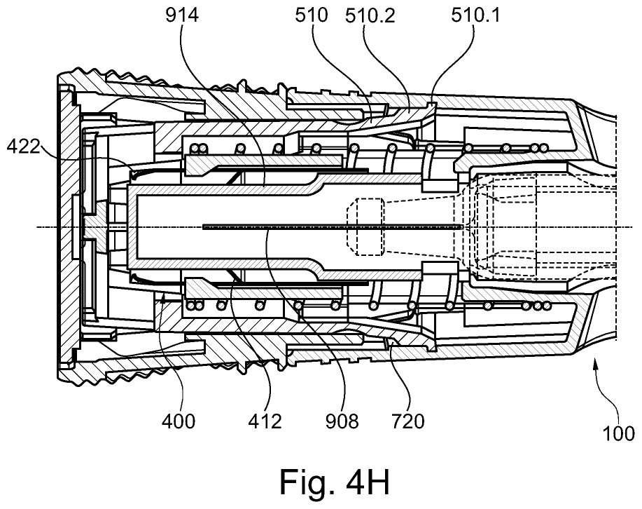

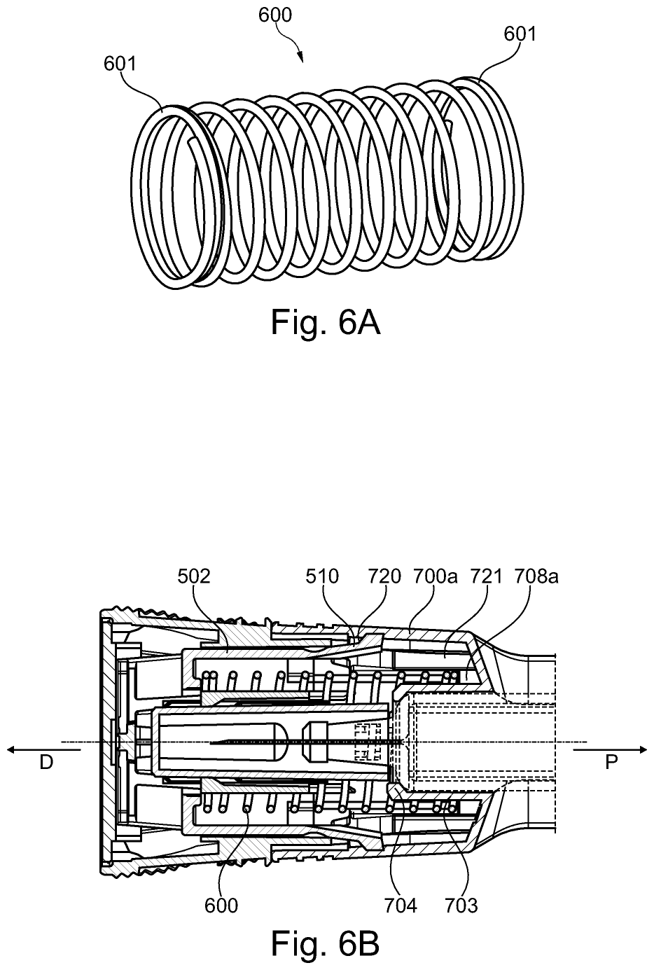

FIG. 3A illustrates the optional cap 200 with the optional cap lid 300 mounted thereon, wherein the cap 200 and the cap lid 300 are detached from the device body 700 and the needle cover 500. The cap 200 may have a different color than the device body 700. The cap 200 shown in FIGS. 3A to 3I may correspond to or be identical with the caps 200 shown in FIGS. 4F, 4H and 6B. The material chosen for the cap 200 and/or the cap lid 300 may be Bayblend M850XF, a medical grade PC/ABS blend. PC/ABS may be chosen primarily for its strength, flexibility and for its strength at high temperature-allowing a shortened injection molding cycle time and, hence, lower part cost. The cap 200 comprises a cap body 201. The cap body 201 (e.g. when seen in plan view) has the shape of a truncated cone, with the radial and/or circumferential dimension of the cap body 201 increasing along the longitudinal axis A in the distal direction D. This shape supports the user in gripping the cap 200 and pulling it off in the distal direction D, e.g. by a purely axial movement relative to the device body. Viewed from above, i.e. looking along the longitudinal axis A, the cap body 201 can have an oval shape or a rectangular shape with rounded corners. This shape may also support the user in gripping the cap 200 and pulling it off in the distal direction D. Moreover, such a shape can prevent the drug delivery device 100 from rolling away when put down by the user. To further facilitate handling for the user, especially when removing the cap 200, the cap 200 features a gripping surface 202, e.g. a side surface of the cap 200. The gripping surface 202 can have a ribbed, flared, square geometry. As shown in FIG. 3A, the cap 200 has at least one cap-user-indicator 203 on its surface. For example, the cap 200 has two cap-user-indicators 203, which are arranged opposite each other. The cap-user-indicator 203, in the depicted embodiment, has the shape of an arrow pointing in the distal direction D. In the proximal direction before the onset of the arrow, the cap 200 can have a rectangular recess. The cap-user-indicator 203 and a body-user-indicator 733 (as described in section 7 below) may form a user-indicator. The cap-user-indicator 203 thus shows the user in which direction the cap 200 must be pulled when removing it from the drug delivery device 100. Since the arrow is designed as a recess in the cap surface, the arrow also supports the user's secure grip when gripping and pulling the cap 200. Thus, the cap-user-indicator 203 provides both a visual and haptic aid for the user. Furthermore, the cap 200 comprises at least one cap clip 204 to connect or mount the cap 200 to the needle cover 500 and thus to the device body 700. As depicted in FIGS. 3B, 3C, 3F, 3H and 3I, the cap clips 204 may be designed as elastic components with free ends in the proximal direction that engage a corresponding part of the needle cover 500. The corresponding part of the needle cover 500 may be the cap clip window 504 as described below. The cap 200, for example, has two opposing cap clips 204. As depicted in FIGS. 3B, 3C, 3F, 3H and 3I the cap clips 204 are an integral part of the cap 200. The cap clips 204 have a proximal free end which delimits the clips 204 in the proximal direction. This free end has an inwardly directed hook or retaining element in the radial direction. The retaining element is designed to engage in the cap clip window 504 of the needle cover 500. The cap 200 has two diametrically opposite cap recesses 213. The recesses 213, in the depicted embodiment, are on the same side as the arrow (see FIG. 3I). For example, the recesses 213 have the shape of a trapezoid (when seen in plan view). The width of the respective recess increases in the proximal direction.

In addition, the cap 200 comprises at least one, for example, more than one, e.g. four, anti-rotation ribs 205. The anti-rotation ribs 205 may assist in enabling fitting of the cap 200 to the device body 700 in only one orientation so that the body-user-indicator 733 and the cap-user-indicator 203 are rotationally aligned and the combined indicator is axially oriented. When the cap 200 is attached to the drug delivery device 100, the anti-rotation ribs 205 prevent accidental rotation of the cap 200 relative to the housing body 700 which could lead to damage or needle-shield coring as the needle shield is expediently rotationally locked to the cap. As can be seen for example in FIGS. 3A, 3H and 3I the anti-rotation ribs 205 may be elongated bars extending along the longitudinal axis of the cap 200 or the device in a proximal direction. The anti-rotation ribs 205 may be integral protrusions of the cap 200. Each anti-rotation rib 205 may be chamfered at its proximal end so that the anti-rotation rib 205 has a ramp at its proximal end.

The anti-rotation ribs 205 may be designed as elongated (e.g., having their main extension along the longitudinal axis A) locking lugs that slide into corresponding recesses or cap groove 725 in the device body 700 when the cap 200 is joined to the device body 700. Thus, it is not possible to rotate the cap 200 relative to the device body 700, i.e. the drug delivery device 100. The cap-user-indicator 203 may be on the same face as the anti-rotation ribs 205 and the cap-recess 213.

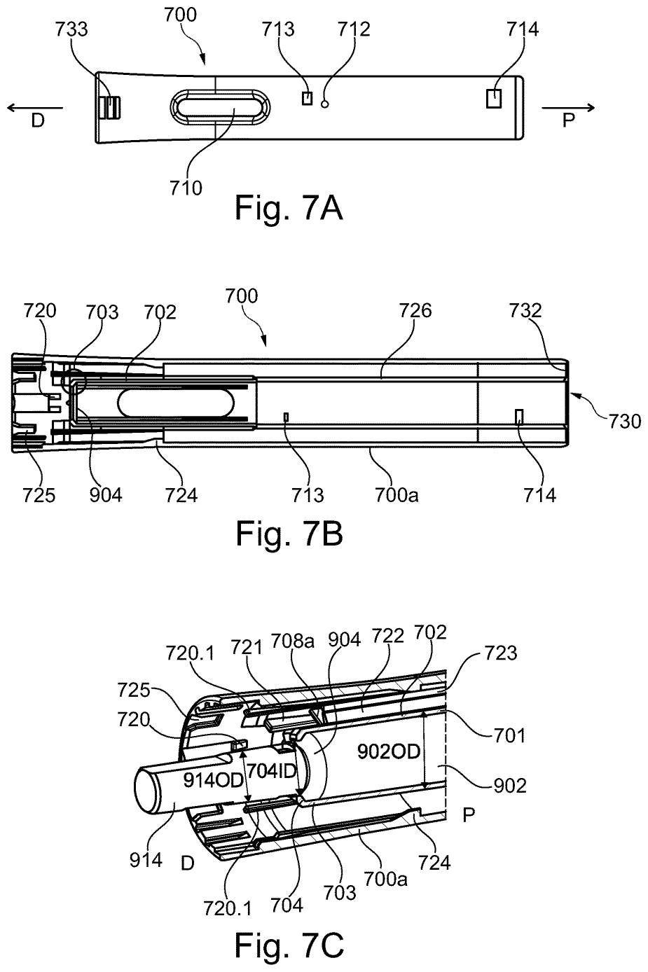

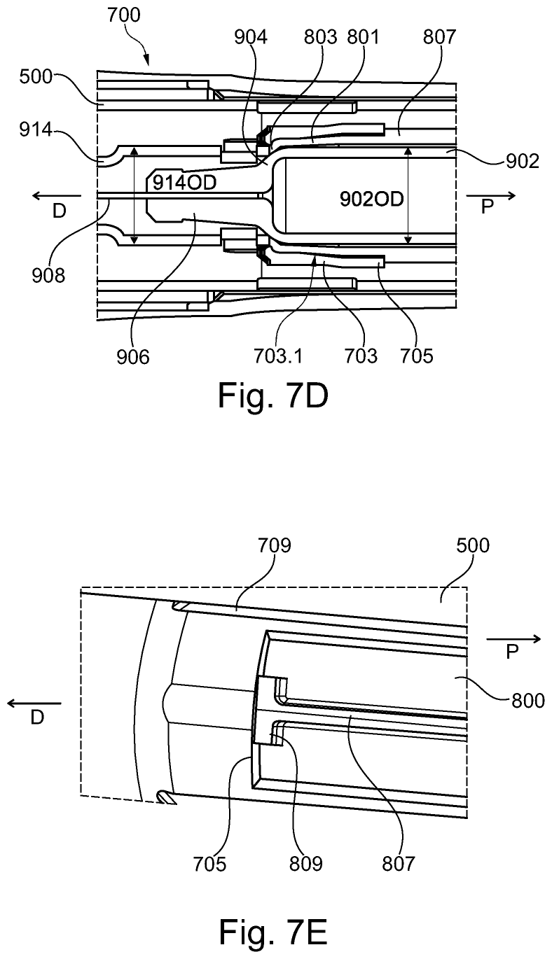

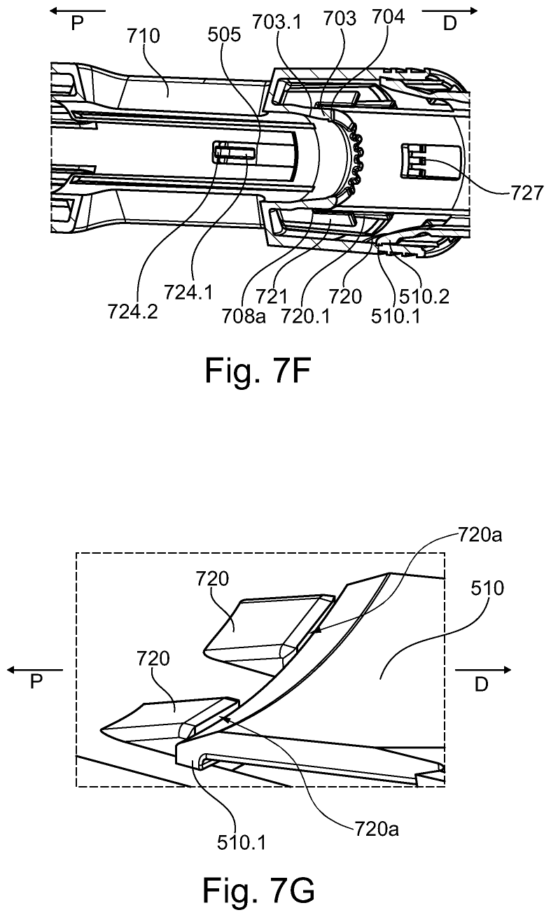

As shown in FIGS. 3B and 3C, the cap clip 204 can help to implement a drop protection, i.e. a drop protection mechanism. In the depicted embodiment the device 100 comprises two cap clips 204. The drop protection mechanism expediently prevents the drug delivery device 100 from firing in the event of it being dropped. Without this mechanism in place, if the drug delivery device 100 is dropped with the cap 200 upwards, the needle cover 500 may continue to move under its own inertia when the drug delivery device 100 hits the ground from the first cover position X (see FIG. 1B) to the second cover position Y (see FIG. 1C), allowing the device 100 to fire. This is prevented by the cap clips 204. FIG. 3B shows the drug delivery device 100 in its pre-use state, wherein the needle cover 500 is in the first cover position X biased forward by the needle cover spring 600. The cap clips 204 sit in a corresponding cap clip window 504 of the needle cover 500 and, as depicted in FIG. 3C, limit a rearward motion of the needle cover 500 in proximal direction P. Thus, the needle cover 500 cannot reach the second cover position Y if the cap clips 204 sit in a corresponding cap clip window 504. The cap clips 204 are restrained by cap ribs 727 on the device body 700 (see FIG. 7F) to prevent them from deflecting outwards. When the cap 200 is removed by the user, the cap 200 first moves forwards, allowing the cap clips 204 to move into a wider section of the device body 700 before the cap clips 204 come into contact with the needle cover 500, enabling the cap clips 204 to deflect outwards and the cap 200 to be removed.

FIGS. 3D and 4F show the interior of the cap 200. The cap 200 has a cap opening 206 for receiving the grabber 400. The cap opening 206 extends distally to a cap case or cap tube 210. The cap tube 210 can have a cylindrical shape. The cap opening 206 may be defined by the cap tube6. The cap opening 206 may be at the proximal end section of the cap tube 210. A plurality of grabber retention bosses 207 (for example two grabber retention bosses 207) are disposed, e.g. equidistantly, along the inner circumferential surface of the cap tube to prevent movement of the grabber 400 relative to the cap 200 in the proximal direction P. The retention bosses are positioned distally from the cap (tube) opening 206. Regarding the interaction between the cap opening 206 and the grabber 400, reference is made to the description of FIGS. 4A-4H. As can be seen in FIG. 3D, the cap has several apertures, distal holes 208 or device priming holes in the longitudinal direction. These holes 208 can serve as access for a tool to prime the device. Priming may involve bringing the device into a condition in which it can be triggered. Priming may involve movement of the needle cover and be a step conducting during assembling, e.g. close to the end of the assembling of the device (which is described further below).

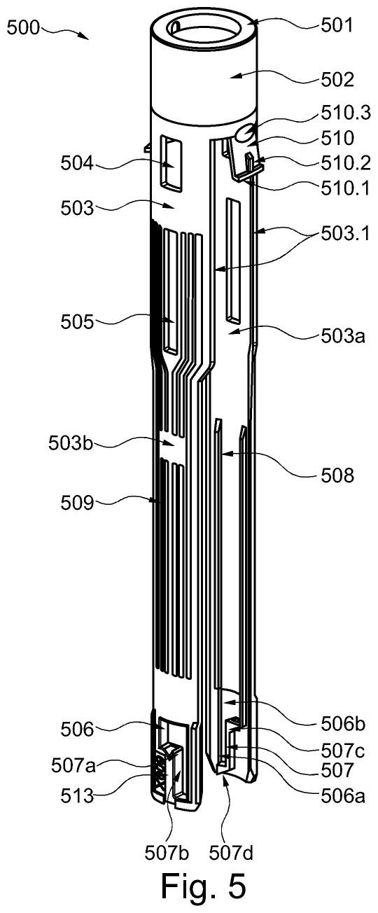

Further, the cap 200 comprises at least one, for example, two, cap-lid clips 209 to join the cap lid 300 to the cap 200. For example, the two cap-lid clips 209 are arranged opposite each other. The cap 200 may have surfaces 214 for an interference fit of the cap lid 300. As can be seen for example in FIGS. 2, 3A, 3B, 3C, 3E and 3F, the cap lid 300 can be arranged in the distal end region of the cap 200. FIG. 3E shows a lateral sectional view of the cap lid 300. The cap lid 300 has a cap lid outer surface 301 which closes the cap 200 in the distal end region. Thus, the distal holes 208 of the cap 200 can be sealed (rendered inaccessible). Another function of the cap lid 300 is to protect the user from re-applying the cap to a drug delivery device 100 that has already been used, wherein this is implemented with the aid of an anti-recapping mechanism. The cap lid 300 further comprises an inner surface facing the proximal direction P. The inner surface has at least one cap lid spacer 302. For example, as depicted in FIGS. 3E and 3F, the cap lid 300 comprises two cap lid spacers 302. The cap lid spacers 302 are arranged opposite each other. Furthermore, the distance between the two end regions of the cap lid spacers 302 pointing in the proximal direction P corresponds at least essentially to and/or is adjusted to the distance between diametrically opposite points on the skin contact surface 501 of the needle cover 500 (see FIG. 5). In other words, the distance between the cap lid spacers may be between an inner diameter of a ring defining the ring-like skin contact surface and an outer diameter of the ring. As shown in FIG. 3F, once the drug delivery device 100 has been removed from the injection site and the needle cover 500 has been brought to its final locked position (see FIG. 1D) after the device has been used or fired, the anti-recapping mechanism prevents the cap 200 from being placed back on the used drug delivery device 100. Since the needle cover 500 protrudes further distally from the device body 700 after dispensing (see FIG. 1D) than before dispensing (see FIG. 1B), and since the distance between the cap lid spacers 302 is chosen appropriately, attempting a reattachment of the cap 200 to the device body 700, as shown in FIG. 3F will result in direct contact between the needle cover 500 and the cap lid 300 before the cap has been connected to the remainder of the device. This means that the cap lid 300, i.e. the cap lid spacers 302, comes into contact with the needle cover 500, i.e. the skin contact surface 501, before the cap 200 can be fully seated on the device body 700. Since the anti-recapping mechanism prevents the cap 200 from being placed on the drug delivery device 100 in the same way as it was initially by a cooperation of the needle cover and the cap lid, the external appearance of the drug delivery device 100 with the cap 200 having attempted to be replaced also differs, so that a used drug delivery device 100 with the cap 200 is visually and tactilely distinguishable from an unused drug delivery device 100 with the cap 200. Advantageously, the cap can no longer be placed on the device body at all are at least not into a position, which the cap had initially, i.e. before the cap was separated from the reminder of the device for use of the device.

As depicted in FIG. 3G, the cap lid 300, for example, has two oppositely disposed positioning structures or positioning arcs 303. For example, each of the positioning arcs 303 has a positioning guide 303a in the end area which is designed as an integral protrusion of the positioning arc 303. The cap lid 300 thus has four positioning guides 303a. The cap lid 300, for example, has interference fit ribs 303b in the middle of the respective positioning arcs 303a. For example, each positioning arc 303 has three ribs 303b. The ribs 303b are configured to form an interference fit with the corresponding interference fit surfaces 214 of the cap 200. Moreover, the cap lid 300, for example, has two oppositely disposed recesses 304.

### 4. Grabber (FIGS. 4A to 4H)

FIGS. 4A to 4H illustrates the optional grabber 400. The cap 200 may be adapted to form part of a needle shield remover or removal assembly. For this purpose, the cap 200 and the grabber 400 may be connected in such a manner that removal of the cap 200 together with the grabber 400 from the drug delivery device 100 removes the needle shield 914 from the needle 908. In other words: The grabber 400 may be coupled to the cap 200 in a manner such that, when the cap 200 is removed, the needle shield 914 is also removed from the needle 908. The grabber may be axially locked to the cap.

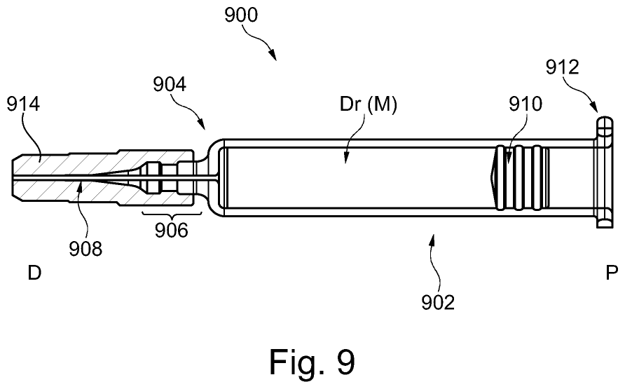

FIGS. 4A and 4B illustrates a perspective view of the optional grabber 400 and a cross section of the grabber 400, respectively. The grabber 400 may be a sheet metal component which sits inside the cap 200 and removes the needle shield 914 from the pre-filled syringe 900 during cap removal. The needle shield 914 (shown e.g. in FIG. 9) can be a rigid needle shield (RNS) or a soft needle shield (SNS).

The grabber 400 in this example may be formed of a single piece, e.g. a sheet such as a metal sheet or a metal alloy sheet (see e.g. FIG. 4C). The grabber 400 may comprise at least a main body or grabber carrier 402. The grabber carrier 402 may be multiply bent or kinked along a plurality of longitudinal fold edges, kinks or bends 404 to form a plurality of carrier portions 406. The respective carrier portions 406 may have or comprise a plan outer surface region or an essentially plan outer surface region.

Furthermore, the grabber carrier 402 plan outer surface region can be bent or angled in such a manner that the outer carrier portions 406 are partly overlapping in an overlapping area 408. Hence, in the bent state the grabber carrier 402 may have a pipe-form or tube-form with e.g. a polygonal cross section. Other cross sections are possible as well, e.g. circular cross sections. The partly overlapping area 408 in the bent state of the grabber carrier 402 may allow compensation of manufacturing tolerances of the grabber 400. The grabber carrier 402 may have free longitudinal ends which may be arranged close to the overlapping area 408.

To grip the needle shield 914, more than one of the plurality of carrier portions 406 may comprise a cut-out or opening 410 from which a respective barb 412 may be bent and may project inwards from the inner surface of the grabber carrier 402 and thus of the carrier portions 406. In the assembled state, the inwardly angled barbs 412 may extend in the distal direction D of the grabber 400 and hence of the drug delivery device100.

The barbs 412 may be adapted to deflect and grip the needle shield 914 during assembly of the needle shield 914 into the drug delivery device 100 (see e.g. FIGS. 4D, 4E and 4H) and may be adapted to further grip the needle shield 914 when the cap 200 is being removed from the drug delivery device 100.

The barbs 412 may be designed as hooks or may have a prong-form. For example, the barbs 412 may be inwardly projected from the inner surface of the carrier portions 406 and may comprise on its free end prongs 414. The prongs 414 may be adapted to be abut or to dig into the outer surface of the needle shield 914. The prongs may be designed to form an interference fit and/or form fit and/or force fit during assembling or a positive and/or non-positive connection at least during removal of the needle shield 914 from the needle (e.g. see FIGS. 4D and 4E). According to another aspect, the prongs 414 may be adapted to already dig into the outer surface of the needle shield 914 when the grabber 400 is being assembled to the needle shield 914. That is to say, a form-fit or positive fit may already be applied during the assembling process and not just after commencement of the cap removal process.

According to the present embodiment, the prongs 414 may be configured as double spikes respectively arranged on each barb 412. This configuration may be realized by a concave shape respectively between two prongs 414 per barb 412. Due to the concave shape and thus controlling the distance between the prongs 414, a penetration depth into the surface of the needle shield 914 may be limited. This may be important, when the needle shield 914 is a rubber needle shield where penetration beyond a certain limit could impact sterility by accessing the needle 908.

The openings 410 with the respective barbs 412 may be arranged on a distal portion D6 (e.g. on the distal half) of the grabber carrier 402, while a proximal portion D5 (e.g. the proximal half) of the grabber carrier 402 may comprise the grabber portions 406 without any openings or barbs.

The proximal portion D5 and the distal portions D6 may be substantially the same length seen along a longitudinal direction. The proximal portion D5, may however also be longer than the distal portion D6. The proximal portion D5 may for example be ca. 10 mm and the distal portion may be 9 mm.

The sum of the length of the proximal portion D5 and the distal portion D6 corresponds to the total length of the grabber 400 seen along a longitudinal direction.

The total length of the grabber may for example be between 15 mm and 25 mm, for example 19 mm.

The grabber 400 may have two opposite axial ends, the first or leading end 418 on the distal portion D6 of the grabber carrier 402 and the second or trailing end 420 on the proximal portion D5 of the grabber carrier 402.

The first end 418, e.g. the leading end 418, of the grabber 400 may be the end which is firstly introduced into the cap opening 206 of the cap 200 during the assembling process. The grabber 400 may have an oblique surface region 422 disposed at the leading end 418. The region 422 may be inclined and directed away from the axis as seen from the first end 418.

The region 422 may be designed to interact with the grabber retention bosses 207 which should engage the associated grabber interface feature 410b, e.g. the opening 410b and/or retaining slot 410b (see FIGS. 4F and 4E). The oblique surface region 422 may be aligned angularly with the grabber interface feature 410b and/or the grabber retention bosses 207 which it should engage when it is assembled to the cap 200. Axially the oblique surface region 422 is offset towards the distal end from the grabber interface feature 410b, which is formed by the opening 410 in the embodiment depicted in FIG. 4A. When the grabber retention bosses 207 contacts the surface region 422 during insertion of the grabber 400 into the cap opening 206 and the grabber 400 is guided further into the cap opening 206, an elastic deformation of the grabber 400 in the radial direction may increase, e.g. until the grabber interface feature-opening 410b/retaining slot 410b-engages the grabber retention bosses 207 (see e.g. FIG. 4F). When the engagement is established, the elastic bias of the grabber 400 may be reduced, e.g. until the grabber 400 abuts the cap 200.

In FIG. 4A, the cutout forming the grabber orientation feature 416 is shown. The cutout may have oblique side surfaces 424 delimiting the cutout angularly. Axially, the cutout may be delimited by a surface 426 as seen in the axial direction away from the first end 418 (e.g. in the proximal direction P). The angular extension of the cutout may reduce or decrease with increasing distance from the first end 418. In other words, the cutout may taper towards the second end 420 (e.g. towards the proximal direction P). The surface 426 delimiting the cutout axially may run perpendicularly to the axis A when seen in plan view or top view onto the cutout. The angle of the surfaces 424 relative to the axis may be less than 90°, e.g. 45 degrees or less, when seen in plan view or top view onto the cutout.

The kinked, folded or bent regions 404 may extend along the longitudinal direction of the grabber 400, for example, along the entire axial extension of the grabber 400. Accordingly, at the leading end or leading edge 418, the kinks, folds or bends 404 may define corners 428. The respective corner may be an angled region of the edge of the grabber 400. The edge or corner 428 may be oriented in the axial direction, i.e. face away from the cap opening 206.

The trailing end 420 of the grabber carrier 402 may comprises at least one further cutout 434. The cutout 434 has the function of aiding the assembly head to maintain orientation during assembly, for example of the grabber 400 with the cap.

The trailing end 420 may comprise also smaller indentations 436, these are indentations formed during production of the sheets of metal used later to form the grabber 400.

FIG. 4C shows an exemplary embodiment of a single piece of a sheet 430 which can form the grabber carrier 402. The metal sheet and hence the grabber 400 may comprise two groups of three openings 410a, 410b, 410c each. Each opening 410 may comprise a respective barb 412. Webs between openings 410a, 410b, 410c may be optional, e.g. there may be a common opening for several barbs 412. All barbs 412 may have the same length and/or shape. Alternatively, at least one of the barbs 412 may have a different shape and/or different length compared to the shape and/or length of other barb(s) 412.

The two groups of openings, e.g. 410a, 410b, 410c, may be arranged in such a way on the metal sheet, that when the grabber carrier 402 is formed the two groups of openings 410 and their respective barbs 412 may be substantially positioned opposite to each other. As such, the force applied on the needle shield by the barbs 412 during removal of the needle shield (RNS or SNS) 914 is more homogeneously and/or symmetrically distributed and a better removal of the needle shield may be achieved.

According to an aspect of the present disclosure, the grabber 400 may be produced by the following steps:
providing the grabber carrier 402 in form of the sheet 430, e.g. a metal sheet such as a stamped or punched (punched-out) metal sheet;
forming a plurality of barbs 412 into the grabber carrier 402 by cutting, punching, stamping or die stamping;
multiple bending or kinking the grabber carrier 402 along a plurality of longitudinal fold edges or lines 404 to form a plurality of carrier portions 406 in such a manner that more than one of the plurality of carrier portions 406 may comprise a respective barb 412;
bending the barbs 412 in such a manner that the barbs 412 project from an inner surface of the associated carrier portion 406, e.g. as it is shown in FIGS. 4A and 4B.

The sheet 402 may be a single piece of sheet metal, which may be cut, e.g. by punching or stamping, to form the cutouts or openings 410 and barbs 412 in the cutouts or openings 410.

The sheet may comprise stainless steel, e.g. EN 1.4310, a high strength stainless steel;

The maximal outer diameter of the grabber 400 in an assembled state may depend on the transversal length I1 of the sheet. The maximal outer diameter of the grabber 400 in an assembled state, however may be smaller than the outer diameter of the folded sheet 430, when the two longitudinal edges of the sheet abut. This may be the case because of the presence of the overlapping area 408 of the grabber 400 in its assembled state (see e.g. FIGS. 4A and 4B)

The openings 410 may have a substantial rectangular form. The extension of the openings 410 along a transversal axis of the sheet 420 may represent the wideness or width of the openings 410, e.g. 410a, 410b and 410c.

The barbs 412 may have a smaller width than the openings 410 and may extend longitudinally along at least a third of the respective opening 410.

For example, the barbs 412 may have a length along a longitudinal axis of greater than or equal to 0.5 mm, 1 mm or 2 mm, measured from their proximal end to the distal end to the prongs of the barbs 412

For example, the barbs 412 may have a length along a longitudinal axis of less than or equal to 3 mm, 2 mm or 1 mm. For example, the barbs may have a width of 1 mm, 2 mm or 3 mm.

FIGS. 4D and 4E show the grabber 400 in an engaged position with the needle shield 914 of the syringe 900. All other elements, such as the body or barrel 902 of the syringe 900 and the cap 200 are not depicted in this figure.

As can be seen, the grabber 400 may be arranged on a distal portion of the needle shield 914 such that at least a proximal portion D4 of the needle shield between the proximal end of the grabber 400, e.g. the trailing edge 420, and the proximal end of the needle shield 914 is not covered.

Accordingly, the length of the grabber 400 may be shorter than the length of the needle shield it is intended to grab, such that a proximal portion of the needle shield 914 extends proximally out over the proximal portion of the grabber 400.

The leading edge 418 of the grabber 400 may be aligned with the distal end of the needle shield 914 along a perpendicular plane or it may be essentially be aligned with the distal end of the needle shield 914.

In an assembled position of the grabber 400 with the needle shield 914, the barbs 412 are expediently bent and penetrate the needle shield 914. As can be seen from FIG. 4D, the contact point, e.g. the penetration point, of the barbs 412 with the needle shield 914 may be offset distally with respect to a longitudinal middle point of the needle shield 914. In other words, the grabber 400 may grab the needle shield 914 at a distal portion of the needle shield 914. In even other words, the grabber 400 may interact with the needle shield 914 through the barbs 412 at a substantially combined distance given by the portions D4 and D5 (and the length of the barbs 412) from the proximal end of the needle shield 914.

The proximal portion D5 of the grabber 400 may have the advantage of stabilizing the needle shield 914 during removal of the cap 200 and therefore of the needle 0itself. As such sterility of the needle 110, 908 may be further maintained.

The required size of the grabber 400 depends on the drug delivery device, the pre-filled syringe and, for example, on the needle shield used in the particular drug delivery device and may therefore vary accordingly.

FIG. 4F shows a cutout view of the grabber 400 of the previous embodiment assembled within a cap 200.

As can been seen the grabber 400 is inserted in the cap opening 206 (shown in FIG. 3D), which is configured and/or dimensioned to receive the grabber 400 when it is introduced. The cap opening 206 may be defined by a tubular or sleeve-like portion of the cap 200, which may be dimensioned to receive the grabber 400 in its interior.

Furthermore, for correct orientation of the grabber 400 during assembling within the cap 200, the grabber 400 may comprise the orientation element 416 indicating an assembling orientation. The orientation element 416 may be designed as a tactile indicator or visual indicator or a combination of them. For example, one of the front surfaces of the grabber carrier 402 is profiled, e.g. waved or pronged. The orientation feature 416 may be a cutout as described above in more detail.

The cap 200 may further comprise at least two lugs, bosses or the grabber retention bosses 207 which may be designed to engage one of the openings 410, for example, the middle one 410b, of a group comprising three openings 410. In an assembled state the grabber retention bosses 207 may abut to the respective distal end 432 of the opening 410 and retain the grabber 400 in its position within the cap 200, see FIG. 4G.

In the context of the present disclosure, “angular” may refer to the azimuthal direction, i.e. the direction defined by the azimuthal angle or rotation angle relative to an axis, e.g. relative to the longitudinal axis running through the cap opening 206.

The grabber 400 may be elastically deformed during the assembling process. Here, the grabber 400 is at first is slightly elastically deformed, e.g. on account of the cap opening 206 having a smaller diameter than the non-deformed grabber 400, before the grabber 400 engages the grabber retention bosses 207. Then, the radial elastic deformation is increased. Accordingly, there may be a force acting in the radial direction and this force may tend to enlarge the diameter of the grabber 400 in one or more regions which are angularly offset from the grabber retention bosses 207.

As depicted inter alia on FIG. 3D the cap 200 may comprises at least one, for example, four, grabber guide features 211 and inner distal holes 212.

In an embodiment suitable to reduce or prevent scraping or flake generation the sensitive region of the cap 200 may comprise the inner distal holes 212.

The inner distal holes 212 may extend radially through a section of the cap, e.g. the cap case 210. The inner distal holes 212 may be defined during molding of the cap 200.

The inner distal holes 212 may overlap axially with the grabber retention bosses 207.

The inner distal holes 212 may extend axially in the region distally or offset away from the cap opening 206 from the grabber retention bosses 207, for example, in the entire region up to the end of the receiving space of the cap case 210.

The opening may overlap axially with the grabber retention bosses 207. In some examples, using the inner distal holes 212 has been proven to be advantageous as well with regards to avoiding scrapes or flakes.

It is noted that there is no grabber guide feature 211 in the sensitive region due to the inner distal holes 212 being provided in that region.

The no grabber guide features 211 may be angularly offset from the inner distal holes212 or the sensitive region as depicted.

Therefore, despite of the inner distal holes 212 in the sensitive region, there may still be a grabber guide features 211 established, which guides the interface features of the gripper and the cap into engagement during assembly.

In FIG. 4G the interaction between the grabber retention bosses 207 of the cap 200 and the opening 410b/retaining slot 410 of an exemplary grabber 400 is illustrated more in detail.

The grabber retention bosses 207 comprises at its proximal end an inclined region or sloped section 207a. The, for example, plane, surface of that section or region may form or define an acute angle, e.g. less than 45°, with the longitudinal axis A. At its distal end, the grabber retention bosses 207 may be arranged to interact with a surface 432 of the grabber 400, e.g. a surface 432 delimiting the retaining slot 410b and/or the opening 410b distally. The end surface 207b of the grabber retention bosses 207 which is provided distally, for example, defines or forms an angle with the axis A which is greater than the angle defined with the axis A by the proximal sloped section 207a. For example, the end surface 207b may be oriented perpendicularly relative to the axis A. The proximal sloped section 207a and the end surface 207b may be connected by a connection region 207c which may extend substantially parallel to the axis A.

In an assembled position the end surface 207b of the grabber retention bosses 207, may abut to the surface 432 of the retaining slot 410b and/or the opening 410b distally.

FIG. 4H shows a cross section of a frontal end of an injection device 100 with a mounted cap 200 and a grabber 400 mounted thereon and interacting with the needle shield 914.

As illustrated the barbs penetrate the needle shield 914 in order to grab it and to be able to remove the needle shield 914 by the removal of the cap 200. The length of the barbs 412 may be such, that only a tip of the barbs penetrates the needle shield 914 in order to maintain the sterility of the needle 908.

In this illustration, the grabber guiding features 422 do not contact the needle shield 914, because the latter has at its front end, e.g. its distal end, a diameter smaller than at its proximal end. In embodiments in which the needle shield 914 has a constant diameter from the proximal end to the distal end, the leading edges 422 would contact the outer surface of the needle shield 914 and provide a guiding aid during assembly of the grabber 400 onto the injection device 100 for injection needles that comprise a needle shield which does not change diameter.

### 5. Needle Cover (Needle Shroud) (FIG. 5)

As depicted in FIGS. 1B to 1D, the drug delivery device 100 may further comprise the needle cover 500. The needle cover 500 is illustrated in FIG. 5 in more detail. The needle cover 500 may protrude distally from the device body 700 and/or may be covered by the cap 200 when the cap 200 is attached to the device body 700. The needle cover 500 may be movable relative to the device body 700 from the first cover position X (see FIG. 1B) to the second cover position Y (see FIG. 1C).

The needle cover 500 may be provided to extend beyond the distal tip of the needle 908 which may protrude from the device body 700 before the drug delivery operation is commenced. The needle cover 500 may be movable in the proximal direction P relative to the device body 700. During this movement, e.g. before the needle cover 500 reaches the second cover position Y, the needle 908 may pierce the skin of the user. The needle cover 500 may serve as a trigger member of the drug delivery device 100. The needle cover 500 as trigger member, when displaced proximally from the first cover position X to the second cover position Y, may automatically initialize the drug delivery operation, for example, when it is in the second cover position Y. The needle cover 500 may be maintained in contact with the skin until the drug delivery operation has been completed, which may be indicated by an audible, tactile, and/or visual indication provided by the drug delivery device 100. After completion of the drug delivery operation, the needle cover 500 may be moved distally relative to the device body 700 to a third cover position Z (see FIG. 1D) to cover the tip of the needle 908.

The drug delivery operation of the drug delivery device 100 may be initialized by removing a mechanical lock which prevents movement of the plunger 1000 in the distal direction or by moving plunger 1000 to disengage a mechanical lock via the moving needle cover 500. Alternatively, the needle cover 500 when moved from the first cover position X to the second cover position Y and expediently when in the second cover position Y may only enable triggering of the drug delivery operation. In this case, a separate trigger member, e.g. a trigger button on the proximal end of the device body 700, may be provided to initiate the drug delivery operation. Operating the trigger button to initiate the drug delivery operation may only be possible when the needle cover 500 is in the second cover position Y. In yet another alternative, the needle cover 500 may only be provided to prevent needle stick injuries before and/or after use of the drug delivery device. In this case, the needle cover 500 may be completely decoupled from the drive mechanism 101 and/or not be involved in triggering or enabling triggering of the drug delivery operation at all. In the presently described device, the needle cover acts as trigger member. Thus, no separate trigger member needs to be actuated by the user.

As described in more detail below, the drug delivery device 100 may comprise the needle cover spring 600. The needle cover spring 600 may be operatively coupled to the needle cover 500 in order to move the needle cover 500 into the distal direction D relative to the device body 700 when the drug delivery device 100 is removed from the skin. The force of the needle cover spring 600 has to be overcome in order to move the needle cover 500 in the proximal direction P away from the first cover position X. In the final or third cover position Z (see FIG. 1D) after the drug delivery operation has been completed, the drug delivery device 100 has been removed from the skin and the needle cover spring 600 has displaced the needle cover 500 distally, the needle cover 500 may be locked against proximal movement with respect to the device body 700.

FIG. 5 shows a detailed exemplary illustration of the needle cover 500. The needle cover 500 may comprise the circular shaped or otherwise shaped skin contact surface 501, which is arranged at a cylindrical distal end portion 502 of the needle cover 500, and is designed to be placed on the user's skin. The skin contact surface 501 may have an opening concentric to the circular shape that extends axially through the cylindrical distal end portion in the proximal direction, wherein this opening encloses the needle 908 in the assembled state of the drug delivery device 100 (when seen in plan view). Starting from the e.g. cylindrical distal end portion 502, the needle cover 500 may have a side region 503 extending in the proximal direction P. In the example shown, the needle cover 500 may have two side regions 503, but it should be noted that the needle cover 500 may have more than two, such as three or four side regions 503, and the respective side regions 503 may have all of the features of the side region 503 described below, respectively. The two side regions 503 are arranged opposite from each other and are designed to enclose the optional syringe holder 800 if present, the pre-filled syringe 900, the plunger 1000 and/or the drive spring 1100 when the drug delivery device 100 is assembled. The side regions may be legs.

Each of the two side regions 503 comprises an inner side region surface 503a and an outer side region surface 503b, wherein the inner side region surface 503a is facing the longitudinal axis in the radial direction and the outer side region surface 503b is facing away from the longitudinal axis in the radial direction. The side region 503 comprises two lateral edges 503.1, The side region 503 comprises three recesses, a cap clip window 504, a front stop slot 505, and a plunger boss slot 506. In the depicted embodiment, the front stop slot 505 may be located between the cap clip window 504 and the plunger boss slot 506 in axial direction, wherein the cap clip window 504 may be offset from front stop slot 505 in the distal direction and the plunger boss slot 506 is offset from the front stop slot 505 in proximal direction.

The cap clip window 504 may be a (e.g. rectangular) recess into which the cap clips 204 can engage when the cap 200 is mounted to the device body 700. The connection between the cap clip 204 and the cap clip window 504 may prevent axial movement of the needle cover 500 relative to the device body 700. This connection may provide a safety feature that may prevent accidental triggering of the dispensing mechanism, for example, if the drug delivery device 100 is accidentally dropped down by the user.

The front stop slot 505 may be a (e.g. rectangular) rectangular recess which may interact with a needle cover front stop 724, e.g. with bosses 724 as illustrated in FIG. 7C, located on an inner surface of the device body 700 to define a maximal distal position of the needle cover relative to the device body 700 after an operation of the drug delivery device, e.g. at the end of injection.

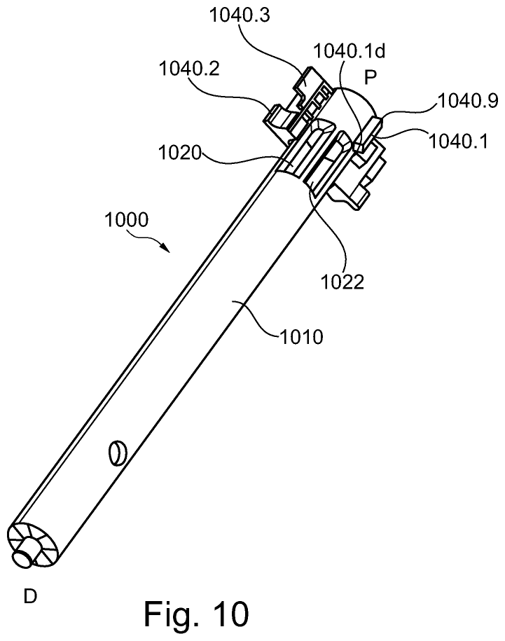

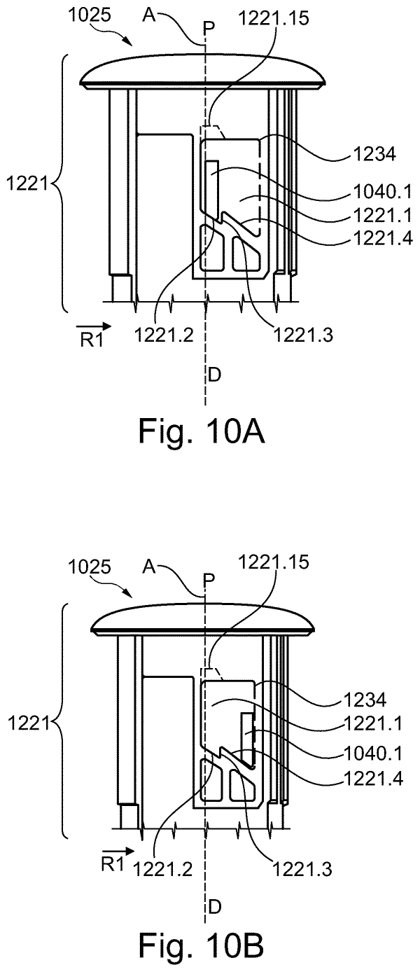

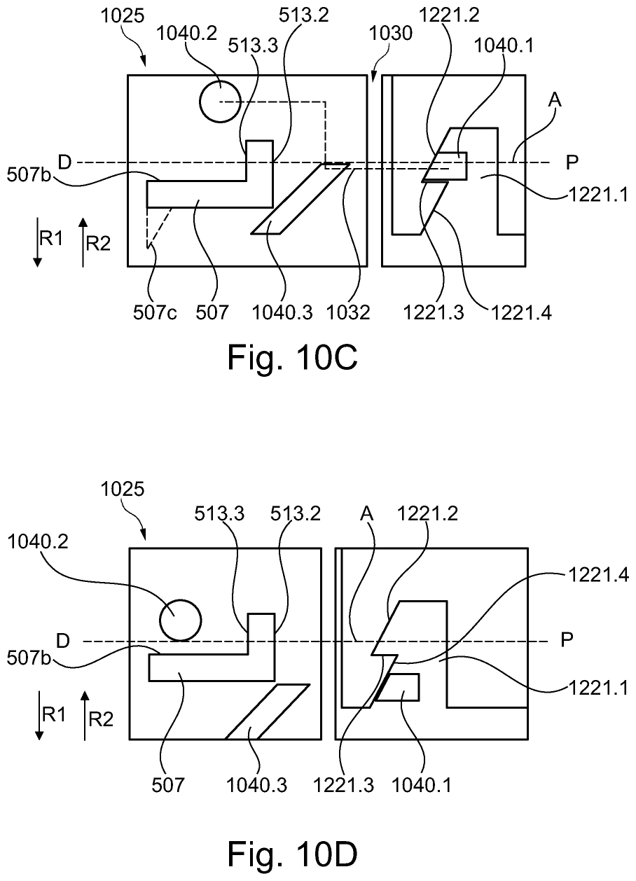

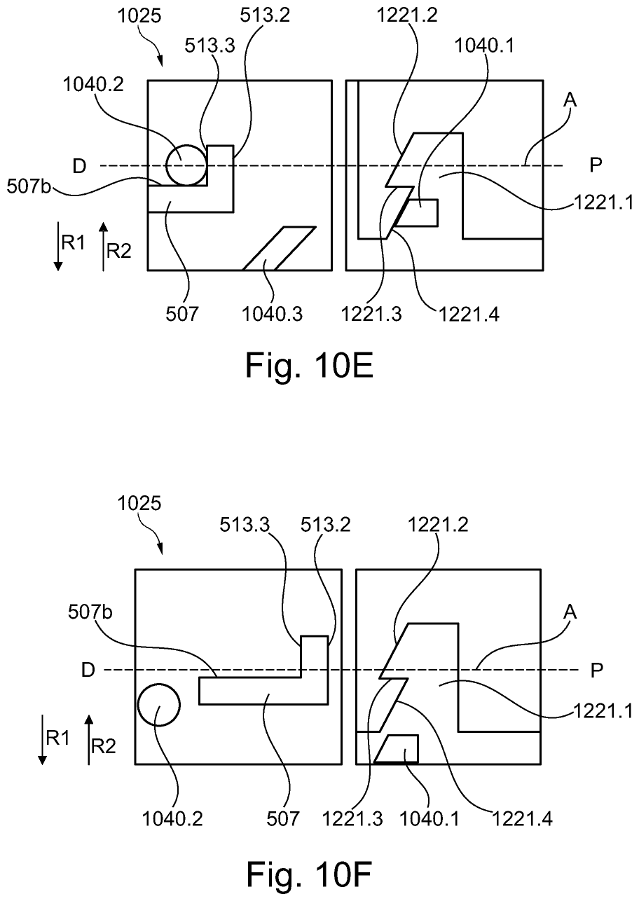

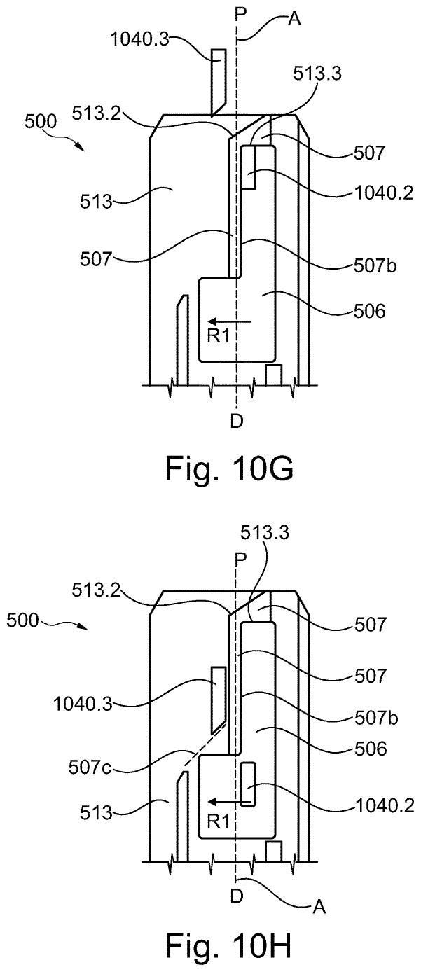

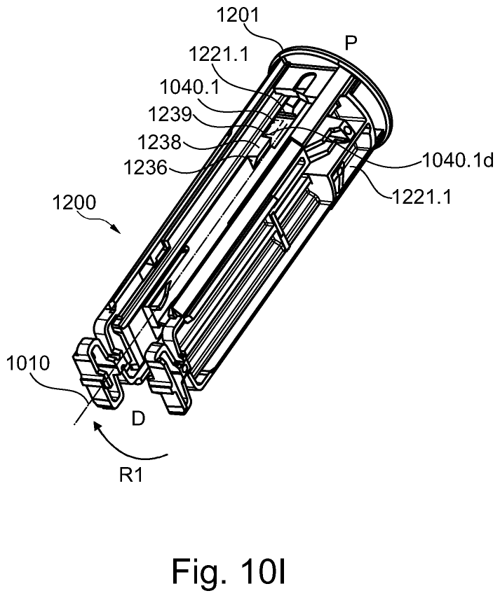

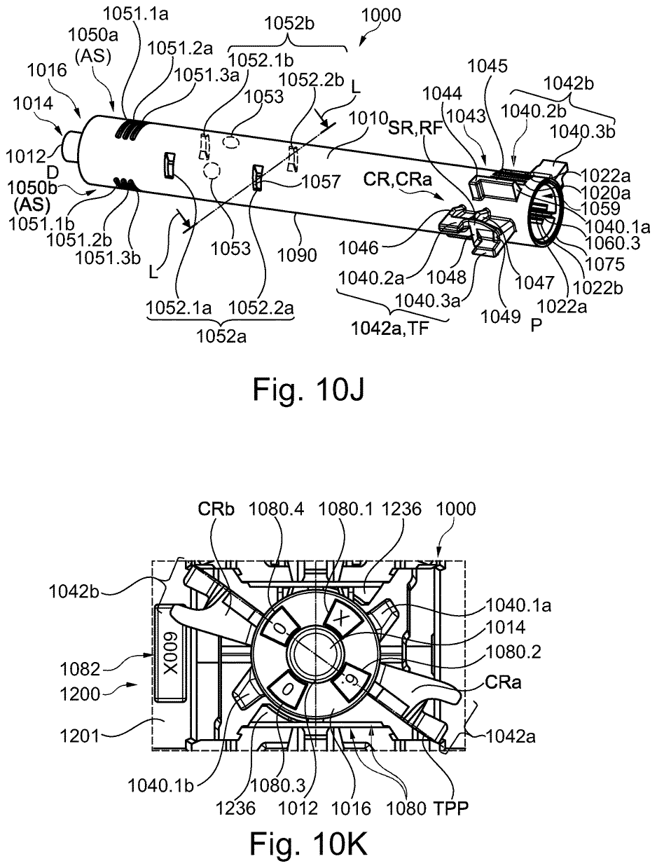

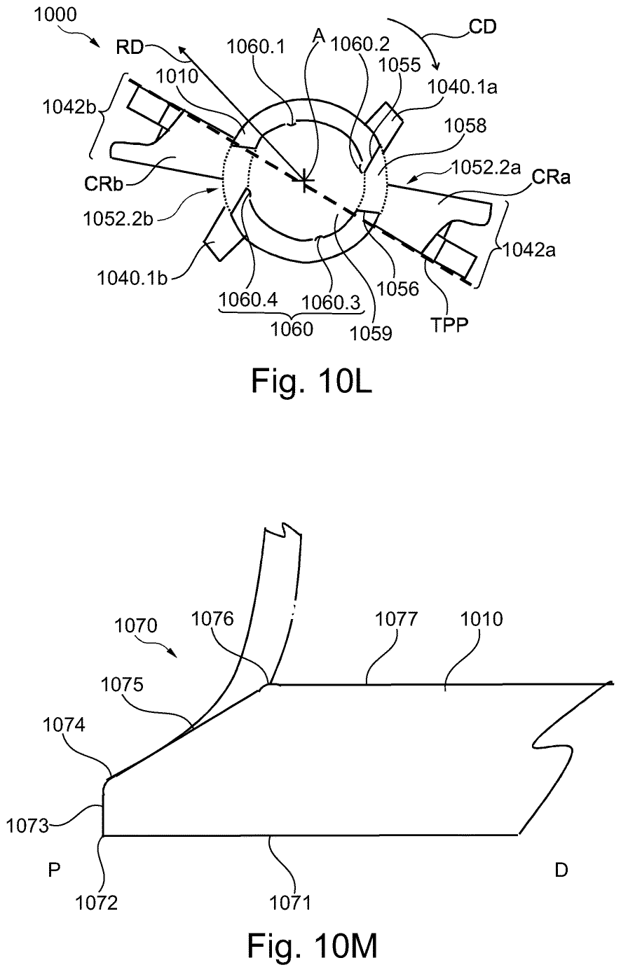

The plunger boss slot 506 may be an L-shaped recess, i.e. a recess formed by two rectangles of different sizes, i.e. a proximal slot 506a and a distal slot 506b, placed directly next to each other. Proximal slot 506a may have a smaller angular width compared to the angular width of distal slot 506b. The plunger boss slot 506 and at least one plunger boss 1040.2, 1040.3 (see FIG. 10) of the plunger 1000 may form a mechanical lock, e.g. a rotational lock. The needle cover 500 may remain in the first cover position X as long as the mechanical lock is established. The plunger boss slot 506 may be configured to allow relative movement of the needle cover 500 to the plunger 1000 when the needle cover 500 is moved from the first cover position X to the second cover position Y in order to release the mechanical lock. Here, due to a plunger boss 1040.2 (see FIG. 10) of the plunger 1000, rotational movement of the plunger 1000 relative to the needle cover 500 may be prevented until the plunger boss 1040.2, 1040.3 (see FIG. 10) is moved in an axial direction from the proximal slot 506a into the distal slot 506b.

The plunger boss slot 506 may comprises a slot rib 507 located on the side of the transition from the proximal slot 506a to the distal slot 506b which may contain a shoulder 507a due to the different rectangle sizes. The slot rib 507 may comprise an abutment surface 507b on the inner side region surface 503a, wherein the abutment surface 507b may be designed for the plunger boss 1040.2 (see FIG. 10) to rest against it. The slot rib 507 may further comprise an optional first ramp 507c and an optional second ramp 507d which may interact with the plunger 1000, e.g. with plunger boss 1040.3 as illustrated in FIGS. 10G and 10H. The optional first ramp 507c may be located at the transition from proximal slot 506a to the distal slot 506b and may interact with the plunger boss 1040.3, see FIGS. 10G and 10H. In the unlikely event that the plunger 1000 does not spontaneously rotate (e.g. when the needle cover is in the second position), the first ramp 507c may interact with the plunger boss 1040.3, see FIGS. 10G and 10H, in order to additionally provoke or initiate the rotation of the plunger 1000. The second ramp 507d may be located at the proximal end of the side region 503 and may be designed to facilitate priming of the plunger 1000 during the final assembly of the drug delivery device 100, e.g. by acting on the plunger boss 1040.3 as illustrated in FIGS. 10G and 10H.

As can be seen in FIG. 5, the needle cover 500 may comprise plunger guide ribs 508 on the inner side region surface 503a that are designed to provide angular guidance to the plunger 1000, e.g. for one of or both of bosses 1040.2 and 1040.3. Furthermore, the needle cover 500 may have grooves 509 on the outer side region surface 503b. The grooves may be provided to reduce part warping during injection molding when manufacturing the needle cover 500 and/or thereafter. Mechanical stability may be raised by grooves 509 as well.

FIG. 5 also shows that the needle cover 500 may have at least one needle cover blocking means 510. The needle cover blocking means 510 may be offset 90 degrees from each cap clip window 504 in the direction of rotation. The needle cover blocking means 510 may be embodied as elastically pivotable flexible arms 510 which are biased or can be biased in a radial direction away from the longitudinal axis in the assembled state of device 100. The needle cover 500 may have several flexible arms 510. For example, the needle cover 500 may have one, two, three, four, five, six, seven, eight or more flexible arms 510. The flexible arms 510 may be evenly distributed along the circumference of the needle cover 500. Furthermore, the flexible arms 510 are arranged opposite to one another. After the drug delivery operation has been completed, the drug delivery device 100 may be removed from the skin of the user. The needle cover 500 may be biased relative to the device body 700 towards the first cover position X by the needle cover spring 600. Thus, when the drug delivery device 100 is removed from the skin the needle cover 500 may be moved towards the first cover position X, e.g. beyond its first cover position X, into the final, locked or third cover position Z relative to the device body 700 as shown in FIG. 1D. In the third cover position Z the needle cover 500 may expediently be axially locked relative to the device body 700 against movement in the proximal direction, by the locking engagement between the flexible arms 510, of the needle cover 500 and associated protrusions of the device body 700, e.g. a needle cover lock structure 720 on an inner surface of a sidewall 700a of the device body 700. The needle cover lock structure 720 may also be referred to as a blocking element 720 or a ramp-like element 720. As it is axially locked, the needle cover 500 can no longer be displaced proximally relative to the device body 700 into the second and/or into the first cover position of needle cover 500. This may protect the user from needle stick injuries after use. In addition, it may no longer be possible to reattach the cap 200 when the needle cover 500 is in the third cover position Z, e.g. due to flexible arms 510 and/or due to features on cap 200, especially on optional cap lid 300.

As shown in FIG. 5, the flexible arm 510 can have a, e.g. cuboidal, elevation 510.1 at its proximal end region in the radial direction. The elevation may protrude radially from the flexible arm. This elevation 510.1 forms the proximal end of the flexible arm 510. The elevation may also be referred to as a stopping surface. In the circumferential direction, the elevation 510.1 may extend further than in the axial direction of the needle cover 500. Due to the cuboid shape and the difference in height in the radial direction relative to the remaining outer surface of the flexible arm, the flexible arm has an edge in the distal direction and in the proximal direction. The elevation 510.1 of the flexible arm 510 can engage a corresponding protrusion at an inner circumferential surface of the device body 700 to lock the needle cover 500 against axial movement relative to the device body 700 (e.g. in the third cover position Z). It should be noted that in the present disclosure, the first cover position X is also referred to as the intermediate position X and the third cover position Z is also referred to as the initial position Z. For example, one of the surfaces of the elevation 510.1, e.g. the surface pointing in the proximal direction P, may abut a distal surface of the needle cover lock structure 720, when the needle cover is in the third cover position Z. Thus, a proximal movement of the needle cover 500 relative to the device body 700 is limited. In other words, the elevation 510.1 and the needle cover lock structure 720 provide an after-use needle cover lock mechanism, which may prevent injury of the user, by preventing exposure of the needle tip.

For example, the after-use needle cover lock mechanism may prevent a proximal movement of the needle cover 500 with respect to the device body 700, if a proximal force applied to the needle cover 500 is less than 60N, for example, less than 50N e.g., less than 40N.

In the present disclosure, the elevation 510.1 may also be referred to as a stop surface 510.1. The flexible arm 510 can also have a web 510.2, as shown in FIG. 5. In the present disclosure, the web 510.2 may also be referred to as a protrusion 510.2. The web 510.2 may have a free end in the distal direction, the free end may be chamfered. The opposite proximal end of the web may transition into the elevation. The web 510.2 may have a lower height or an identical height in the radial direction as the elevation 510.1. The web 510.2 may interact with the needle cover lock structure 720 of the device body 700 (see also FIG. 4H). For example, the web 510.2 may interact with a recess of the needle cover lock structure 720, in order to prevent rotational movement of the needle cover 500 relative to the device body 700. Alternatively or additionally, due to the interaction, there may be less inward deflection of the flexible arm 510 necessary when the flexible arm 510 moves along the needle cover lock structure 720, e.g. when the needle cover 500 moves from its second cover position Y to its third cover position Z relative to the device body 700. This reduces the load on the flexible arm.

As shown in FIG. 5, the flexible arm 510 has a recess 510.3 at its interface with the rest of the needle cover body. The circular recess 510.3 may be a circular material recess. The material recess provides a hinge region between the flexible arm 510 and the rest of the needle cover body. The region of the needle cover adjoining the flexible arm, e.g. the distal end portion 502 may be a cylindrical sleeve like region of the needle cover. In some examples, only this region protrudes from the device body in any one of the positions of the needle cover relative to the device body.

Furthermore, it may be possible for the needle cover 500 to move slightly in the distal direction immediately after the cap 200 is removed but before the needle cover 500 is placed on the skin surface and before the plunger 1000 and the energy of the drive spring 1100 is released. In this case, the needle cover 500 slides forward slightly, since the needle cover spring 600 is released and the plunger 1000 rotates to a position where it is ready for use. For example, this may be the case when the cap 200 is directly engaged with the device body 700 of the drug delivery device 100 or the device body 700 and the needle cover (biased by the needle cover spring) abuts the cap before it is being removed.

It is noted that all features of the needle cover 500, i.e. in the skin contact surface 501, distal end portion 502, side region 503, inner side region surface 503a, outer side region surface 503b, cap clip window 504, front stop slot 505, plunger boss slot 506, proximal slot 506a, distal slot 506b, slot rib 507, shoulder 507a, abutment surface 507b, first ramp 507c, second ramp 507d, plunger guide rib 508, grooves 509, and flexible arm 510, may be or are integral part of the needle cover 500. Accordingly, the needle cover 500 with all its features may represents a one-piece component. However, a two-part or a multi-part needle cover 500 may be used as well.

### 6. Needle Cover Spring (FIGS. 6A, 6B)

FIGS. 6A and 6B illustrate a needle cover spring 600. As illustrated in FIG. 6B, the needle cover spring 600 may extend between the needle cover 500 and a device body 700 (described below). For example, the needle cover spring 600 may extend between a proximal facing surface of the needle cover 500 and a distal facing surface of a central support structure 701 of the device body 700. In some examples, the needle cover spring 600 may extend between a proximal facing inner surface of the distal end portion 502 of the needle cover 500 and a needle cover spring support 708a of the device body 700, as described below.

In one embodiment, the needle cover spring 600, e.g., a proximal end thereof, may be supported in a radial outward direction by a needle cover back stop 721 (described below) of the device body 700. In other words, tilting of the needle cover spring 600 relative to the device body 700 and/or a radial outward deflection of the needle cover spring 600 may be prevented by the needle cover back stop 721.

The needle cover spring 600 may be configured to provide a force to the needle cover 500. This force biases the needle cover 500 in the distal direction D. The needle cover spring 600 may be configured such that its force may push the needle cover 500 distally, when a force smaller than the force of the needle cover spring is applied to the needle cover in the proximal direction P, e.g. when the drug delivery device 100 is removed from the skin of the user after injection, or when the injection is interrupted. Moreover, the needle cover spring 600 may be configured such that it ensures that the device 100 can only be activated, if the needle cover 500 is pressed against the user's skin with a sufficiently large force. In other words, if the user does not press the needle cover 500 against the injection site with a sufficiently large force, the drug delivery device 100 is not activated/triggered. Hence, the needle cover spring 600 may be configured such that a minimum activation force requirement is met. For example, the minimum force required to activate the drug delivery device may be between 1 N (Newton) and 50 N, for example, less than 20 N.

In one embodiment, the needle cover spring 600 may be made of a high strength stainless steel. For example, the needle cover spring 600 may be made of an austenitic steel with sufficient elastic properties allowing for an elastic compression of the needle cover spring 600. In one embodiment, the needle cover spring 600 may be made of an austenitic chromium-nickel steel. In one embodiment, the needle cover spring 600 may be made of a DIN EN 1.4310 steel.

In one embodiment, a length of the needle cover spring 600 may be chosen such that a flat force profile can be achieved and an excessive activation force can be avoided.

In one embodiment, the needle cover spring 600 may be made of a coiled wire. A wire diameter may be selected according to the stresses that occur when the needle cover spring 600 is compressed. Further, the wire may be a soap lubricated wire in order the to aid manufacturability.

In one embodiment, the needle cover spring 600 may have between 5 and 50 coils, for example, between 5 and 25 coils e.g., 10 coils. An outer coil diameter may be chosen in accordance with the geometry of the needle cover 500, e.g., the flexible arms 510 of the needle cover 500, such that collisions with the flexible arms 510 can be avoided when the flexible arms 510 are deflected over a needle cover lock structure whilst the needle cover spring 600 surrounds an axial support front end 703 of the device body 700.

In one embodiment, the outer coil diameter may be between 5 mm and 20 mm, for example, between 10 mm and 15 mm, between 12 mm and 14 mm, e.g. 13 mm. An inner coil diameter may be between 5 mm and 20 mm, for example, between 10 mm and 15 mm, between 11 mm and 13 mm, e.g. 12 mm.

In one embodiment, the coiled wire may have a double or triple winding 601 at its ends. The double or triple winding 601 may be formed by two or three coils axially contacting each other along their circumference. The double or triple winding 601 may provide a contact surface for securely contacting the needle cover spring 600 with the needle cover 500 and the device body 700.

In one embodiment, the needle cover spring 600 may have a length between 30 mm and 100 mm. In one embodiment, the needle cover spring 600 may have a length between 50 mm and 80 mm. In one embodiment, the needle cover spring 600 may have a length between 60 mm and 70 mm, for example, 66 mm.

### 7. Device Body (FIGS. 7A to 7G)

FIGS. 7A and 7B illustrate a device body 700 according to an embodiment of the disclosure. The device body 700 may be the main housing of the drug delivery device 100. The device body 700 may provide a space for housing some or all of the components of the drug delivery device 100.

The device body 700 may have a cylindrical shape. In other words, the device body may have a distal end and a proximal end which are connected to each other by a sidewall 700a. The sidewall 700a defines a cross-sectional area of the device body 700. The cross-sectional area may be substantially constant along the axial length of the device body 700. Optionally, towards the distal end, the cross-sectional area may increase such that at the distal end the cross-sectional area may be larger than at the proximal end.

In one embodiment, the cross-sectional area may increase from a drug window 710 (sidewall window) in the sidewall 700a, e.g. a middle thereof, until the distal end. The increase may be linear or non-linear, e.g. parabolic, such that an outer surface of the sidewall 700a of the device body 700 may be curved towards the distal end.

At its proximal end, the device body 700 comprises a proximal aperture 730. The proximal aperture 730 may be defined by a proximal edge 732 of the sidewall 700a.

The sidewall 700a may provide a user gripping surface allowing the user to handle and/or operate the drug delivery device 100.

The sidewall 700a of the device body 700 may comprise at least one opening. The opening may be the drug window 710. The drug window 710 may be arranged in a distal half of the sidewall 700a, for example, in a fourth and/or a fifth section thereof, when assuming an axial length of the sidewall 700a measured from the proximal end to be divided in six equally long sections. The drug window 710 may be an elongated window, which extends longer in an axial direction than in a circumferential direction. On an outer surface, the sidewall may further comprise a portion for labelling.

Through the drug window 710, the user may be able to see a plunger stopper 910 of a syringe 900 (described below) and/or the plunger 1000 (described below). Through the drug window 710, the user may further see the drug Dr before and during injection. For example, during operation of the drug delivery device 100, the user firstly may see the drug Dr and a barrel 902 of the syringe 900 (described below), which may be a pre-filled syringe containing the drug Dr. During injection, the user may see the plunger stopper 910 and then the plunger 1000 moving inside the barrel 902 in a distal direction D.

An outer surface of the sidewall 700a and/or an inner surface of the sidewall may comprise interaction elements for supporting other components of the drug delivery device 100, e.g. the cap 200 and/or the needle cover 500 and/or a syringe holder 800 (described below) and/or a drive spring holder 1200 (described below). The interaction elements may be predominantly arranged on the inner surface of the sidewall 700a and may comprise elements such as ribs, grooves, protrusions, notches and the like, which enable a physical interaction with corresponding features of the other components of the drug delivery device 100.

In one embodiment, the sidewall 700a may further comprise a first body connection structure. The first body connection structure may comprise at least one, for example, at least two recesses. As illustrated in FIG. 7A, the recesses may be proximal cut-outs 714. The proximal cut-outs 714 may be arranged proximally offset from the drug window 710, e.g. close to the proximal end of the device body. For example, the proximal cut-outs 714 may be arranged in the first 20 percent or in the first 10 percent of the length of the sidewall 700a, when measured from the proximal end. The proximal cut-outs 714 may be offset from each other by 180 degrees in the circumferential direction of the device body 700. At least one of the proximal cut-outs 714 may overlap with the drug window 710 in the circumferential direction.

As described below, e.g. in section 12, the proximal cut-outs 714 may interact with snap protrusions 1203.2 of snap arms 1203 of the drive spring holder 1200, e.g. when the drive spring holder is in a (first) drive spring holder position (closure part position). Further, as described below, the proximal cut-outs 714 may interact with holding clips 806 of the syringe holder 800, e.g. when the syringe holder 800 is in a first syringe holder position (first container holder position).

The sidewall 700a may further comprise an injection molding gate recess 712. The injection molding gate recess 712 may be formed at an outer surface of the sidewall 700a. The injection molding gate recess 712 may not penetrate the sidewall 700a. The injection molding gate recess 712 may be distally offset from the proximal cut-outs 714. The injection molding gate recess 712 may be proximally offset from the drug window 710. In one embodiment, the injection molding gate recess 712 may be arranged near to or at the middle of the sidewall 700a in the axial direction of the device body 700.

In one embodiment, the sidewall 700a may further comprise a second body connection structure. The second body connection structure may comprise at least one, for example, at least two recesses. As illustrated in FIG. 7A, the recesses may be distal cut-outs 713. The distal cut-outs 713 may be arranged proximally offset from the drug window 710. The distal cut-outs 713 may be distally offset from the injection molding gate recess 712. The distal cut-outs 713 may be distally offset from the proximal cut-outs 714. In one embodiment, the distal cut-outs 713 may be arranged at the middle or close to the middle of the sidewall 700a in the axial direction. The distal cut-outs 714 may be angularly offset from each other by 180 degrees.

The proximal cut-outs 714 may be larger than the distal cut-outs 713. In other words, the proximal cut-outs 714 may further extend in at least in one spatial direction than the distal cut-outs 713. In one embodiment, at least at least one of the distal cut-outs 713 may be aligned with at least one proximal cut-out 714.

At least one, for example, all, of the distal cut-outs 713 may be overlapped by a corresponding number of proximal cut-outs 714 in the circumferential direction. The overlap can be partially.

For example, the proximal cut-outs 714 fully overlap the distal cut-outs 713 in the circumferential direction. Further, at least one of the distal cut-outs 713 may be overlapped by or aligned with the drug window 710 in the circumferential direction.

As described below, the distal cut-outs 713 may interact with holding clips 806 of the syringe holder 800. Thus, the syringe holder 800 may be secured to the device body 700, e.g. in a second syringe holder position (second container holder position).

The cut-outs 713 and/or 714 may be arranged offset from a center line between the holder guide ribs 726 (described below in more detail). Alternatively, or additionally, the cut-outs 713, 714 may be offset from a longitudinal center line of the drug window 710 extending in the axial direction, as is illustrated in FIGS. 7A and 17. Alternatively, or additionally, the cut-outs 713 and/or 714 may be arranged in the middle, i.e. centered, between holder guide ribs 726.

The device body 700 may comprise a body-user-indicator 733 at an outer surface of the sidewall 700a. The body-user-indicator 733 may be configured to indicate to a user a position of the device body 700 with respect to other components of the drug delivery device 100, e.g. the cap 200.

When the sidewall 700a is labelled, the injection molding gate recess 712 and/or the cut-outs 713, 714 may be hidden by the label, such that at least one of these features, at least two of these features or all of these features are not visible to the user. This may provide comfort to the user.

The device body 700 may be formed of PC (polycarbonate) Makrolon 2258, a medical grade PC or another material. PC may be chosen primarily for its strength, flexibility, toughness and for its strength at high temperature—allowing a shortened injection molding cycle time and hence lower part cost.

As shown in FIG. 7B, certain features of the device body 700 may form a syringe support mechanism. The syringe support mechanism may be formed inside the device body 700. The syringe support mechanism may be configured to position the syringe 900 within the device body 700 such that a needle extension requirement is met. The needle extension requirement may be, for example, that a distal end of a needle 908 of the syringe 900 extends beyond the distal end of the device body by a certain length, e.g. between 4 mm (millimeter) and 8 mm, whilst withstanding an impact load from an impact of the plunger 1000 on a plunger stopper 910 of the syringe 900 at the start of injection.

In one embodiment, the syringe support mechanism is configured to support a syringe, e.g. a pre-filled syringe 900, in the device body 700, e.g. against a distal movement relative to the device body 700. In one embodiment, the syringe support mechanism may be configured to support a shoulder 904 of the syringe 900. Thus, manufacturing tolerances may be compensated better compared to designs in which the syringe 900 is supported at its proximal flange 912. This leads to less variability in the extension of a distal needle end beyond the device body 700, when the syringe 900 is in a final assembled position in the device body 700.

Specific design of the assembly process may be required to ensure that the components reach their correct final positions and the syringe 900 is properly supported.

As illustrated in FIG. 7C, the syringe support mechanism may comprise a central support structure 701 formed at the interior of the device body 700. In one embodiment, the central support structure 701 may be configured to support a barrel 902 of the syringe 900, e.g. at the shoulder 904. In one embodiment, as illustrated in FIGS. 7D and 7E, the central support structure 701 may be configured to support a syringe holder 800.

The central support structure 701 may comprise a central tube 702 configured to radially support the barrel 902 of the syringe 900 (see FIG. 7C) or the syringe holder 800 (not illustrated). In one embodiment, the central tube 702 may have a non-closed circumference, e.g. it may comprise at least one axial recess extending in the axial direction of the central tube 702. The central tube 702 may have an axial length that is more than half or more than three quarters of the axial length of the barrel 902 of the syringe 900, e.g. when measured from the shoulder 904 to the distal surface of the syringe flange 912 (not illustrated).

The central support structure 701 may comprise an axial support front end 703 configured to support the syringe 900 in the axial direction, e.g. against a distal movement relative to the device body 700. The axial support front end 703 may be formed at a distal end of the central tube 702.

In one embodiment, the axial support front end 703 may comprise a radially inward extending protrusion 704 at its distal end. The axial support front end 703 and the radial protrusion 704 may interact with the shoulder 904 of the syringe 900, thereby preventing a distal movement of the syringe 900 beyond the axial support front end 703, e.g., beyond the protrusion 704. In other words, the axial support front end 703 may define the maximum distal position of the syringe 900 relative to the device body 700 and hold the syringe 900 in its desired axial position relative to the device body 700.

In one embodiment, the axial support front end 703 may have a closed circumference. Thus, the axial support front end 703 may surround the shoulder 904. The closed circumference may enable a homogenous distribution of forces over the whole contact surface. It further may enable the axial support front end 703 to withstand higher loads and impacts in the distal and/or radial direction.

In one embodiment not illustrated in the Figures, the axial support front end may have a non-closed circumference, e.g. interrupted by at least one recess extending in the axial direction. In this embodiment, axial support front end 703 may be thicker in a radial direction in order to be able to withstand the forces of the drug delivery device 100 during assembly and/or operation, e.g. a force of the drive spring 600.

In one embodiment, the radial inward protrusion 704 may have an inner diameter 704ID which is smaller than an outer diameter 902OD of the barrel 902. For example, the inner diameter 704ID may be at least 2 percent, at least 5 percent, at least 10 percent, or at least 20 percent smaller than the outer diameter 902OD.

In one embodiment as illustrated in FIG. 7C, an outer diameter 914OD of a needle shield 914 (described below) may be smaller than the inner diameter 704ID of the protrusion 704, thereby allowing the needle shield 914 to move distally beyond the protrusion 704. For example, the outer diameter 914OD may be at least 2 percent, at least 5 percent, at least 10 percent, or at least 20 percent smaller than the inner diameter 704ID of the protrusion 704. The outer diameter 914OD of the needle shield 914 may be smaller than an outer diameter 902OD of the barrel 902. Further details regarding the syringe 900 are described in section 9 below.

In one embodiment as illustrated in FIG. 7D, an outer diameter 914OD of the needle shield 914 may be larger than the outer diameter 902OD of the barrel 902, e.g. at least 2 percent, at least 5 percent, at least 10 percent, or at least 20 percent larger. This may require the use of a syringe holder 800 in order to enable the assembly of the syringe 900 in the drug delivery device 100, e.g., in the device body 700. However, a syringe holder 800 may also be used if the outer diameter 914OD of the needle shield 914 is equal the outer diameter 902OD of the barrel 902 or even if the outer diameter 914OD of the needle shield 914 is smaller than the outer diameter 902OD of the barrel 902.

In one embodiment, the axial support front end 703 may be configured to support the syringe holder 800 in an axial direction relative to the device body 700 (see FIG. 7D). For example, the axial support front end 703 may be configured to secure the syringe holder 800 against a distal movement relative to the device body 700. For example, the axial support front end 703 may be configured to surround flexible holder arms 801 of the syringe holder 800. The radial inward protrusion 704 may form an abutment surface for the holder protrusion 803. The axial support front end 703 may have a closed circumference. Thus, the axial support front end 703 may surround the holder arms 801. The axial support front end 703 may have a conical inner surface 703.1 with a reducing diameter in the distal direction D. The conical inner surface 703.1 may be configured to interact with the holder arms 801, when the syringe holder 800 is moved distally relative to the device body 700. Thus, the conical inner surface 703.1 may limit a radial outward movement of the holder arms 801 or even cause the holder arms 801 to deflect radially inwardly. As regards further details of certain interactions between the device body 700 and the syringe holder 800, the above-described interactions between the device body 700 and the syringe 900 apply, and vice versa, if possible from a technical point of view.

The central support structure 701, e.g., the central tube 702 thereof, may comprise at least one central support window 709 (see FIG. 7E). The central support window 709 may be aligned with the drug window 710 or at least partially overlap with the drug window 710 in order to allow inspection of the syringe 900, the drug within syringe 900, the plunger stopper 910 and/or the plunger 1000, e.g. during assembly and/or injection. If a syringe holder 800 is used in the drug delivery device 100, the central support window 709 may be aligned or at least partially overlap with a holder window 808 (described below).

The central support structure 701 is connected to the sidewall 700a of the device body 700 by at least one connecting element. The connecting element may comprise at least one connecting rib 708 extending from an inner surface of the sidewall 700a to an outer surface of the central support structure 701. The connecting rib 708 may further extend in the axial direction of the device body 700. For example, the connecting rib 708 may extend along at least 50 percent, at least 60 percent, or at least 75 percent of an axial length of the central support structure 701. The connecting rib 708 may extend distally until a distal end of the axial support front end 703.

In one embodiment, there may be several connecting ribs 708, e.g. at least two, at least three or at least four connecting ribs 708. The connecting ribs 708 may be arranged equidistantly in a circumferential direction of the device body 700. Alternatively, as illustrated in FIG. 7C, the connecting ribs 708 may be arranged with different angular offsets between each other. For example, two connecting ribs 708 may be connected to each other by a proximal surface and/or distal surface of the drug window 710, which extend radially from the central support 701 to an outer surface of the sidewall 700a. The angular offset between two connected connecting ribs 708 may be less than 90 degrees, e.g. less than 80 degrees, less than 70 degrees or less than 50 degrees. Consequently, the angular offset between two connecting ribs 708 which are not connected by the proximal and/or distal surface of the drug window 710 may be greater than 90 degrees, e.g. greater than 100 degrees, greater than 110 degrees or greater than 130 degrees. The connected configuration was explained exemplarily, only. Similar angular offsets of the connecting ribs 708 are possible, even if the connecting ribs 708 are not connected to each other.

Thus, a contact between the needle cover 500 and the device body 700 may be improved, which may improve the axial and/or rotational stability of the needle cover 500 inside the device body 700.

At a radial inner section at least one, for example, all, connecting ribs 708, may form a needle cover spring support 708a. In this context, the radial inner section is the section of the connecting rib 708 which is connected to the central support structure 701. In other words, it is the section which is farther radial inwards than the section which is connected to the inner surface of the sidewall 700a. The needle cover spring support 708a may interact with the needle cover spring 600, e.g. a proximal end thereof, in order to support the needle cover spring 600 in the axial direction.

The illustrated embodiment comprises four connecting ribs 708 extending in the axial direction of the central support structure 701, e.g. from a proximal end of the central support structure 701 to the axial support front end 703. In other words, the illustrated central support structure 701 may be connected to the sidewall 701a along at least 70 to 95 percent of its axial length.

According to an embodiment which is not illustrated, instead of a central tube 702, the syringe support mechanism may comprise, at least two, at least three or at least four support arms extending in the axial direction of the device body 700. The support arms may be arranged equidistantly or with different angular offsets around the circumference of the central support structure 701. In the circumferential direction, the support arms may extend over various angles, depending on the number of support arms. For example, if there are two support arms, each support arm may cover less than 90 degrees of the circumference of the central tube 702. Hence, an angle between the support arms may be 90 degrees or more. At their distal ends, the support arms may be connected to each other, thereby forming an axial support front end as described in the foregoing. All specifics of this axial support end may be similar to the specifics described in the foregoing with respect to the embodiments having the central tube 702 with the axial support end 703.

In one embodiment, the proximal cut-outs 714 are configured to form a space into which snap protrusions 1203.2 of snap arms 1203 of the drive spring holder 1200 can deflect, when being aligned with the proximal cut-outs 714. In other words, the snap arms 1203 and the cut-outs 714 may form a case snap mechanism.

The case snap mechanism may secure the drive spring holder 1200 to the device body 700, preventing the drug delivery device 100 from being disassembled by the user or under the impact load of the drive spring 1100 at the start of injection, e.g. as the plunger 1000 contacts the plunger stopper 910. For example, when the snap arms 1203 interact with the proximal cut-outs 714, an axial movement of the drive spring holder 1200 relative to the device body 700 may be limited, for example, avoided. Thus, the drive spring holder 1200 may be in a first drive spring holder position, which may be a closure part position. Alternatively, or additionally, when the snap arms 1203 interact with the proximal cut-outs 714, a rotational movement of the drive spring holder 1200 relative to the device body 700 may be limited, for example, avoided. In other words, in the closure part position, the drive spring holder 1200 may be secured to the device body 700 against axial and/or rotational movement.

In one embodiment, if the device body 700 is used for a drug delivery device 100 with a syringe holder 800, the proximal cut-outs 714 may interact with holding clips 806 of the syringe holder 800, when the syringe holder 800 is moved distally into the device body 700 from a proximal end, e.g. through the proximal aperture 730.

As described below, upon alignment of the holding clips 806 with the proximal cut-outs 714, the holding clips 806, deflect radially outwardly into the proximal cut-outs 714 and secure the syringe holder 800 to the device body in a first container holder position. The first container holder position may be a first engaged position of the syringe holder 800, as described below. In other words, the proximal cut-outs 714 provide a space for the holding clips 806, during assembly of the syringe holder 800 in the device body 700

When a distally directed force is applied to the syringe holder 800, the holding clips 806 may deflect radially inwardly, due to an interaction with the inner surface of the sidewall 700a and thereby disengage from the proximal cut-outs 714. Thus, the syringe holder 800 becomes free to move further distally inside the device body 700. When the holding clips 806 align with the distal cut-outs 713, the holding clips 806 may deflect radially outwardly into the space provided by the distal cut-outs 713, thereby locking the syringe holder 800 with respect to the device body 700 in a second container holder position. The second container holder position may be a second engaged position of the syringe holder 800, as described below.

In one embodiment, the device body 700 may comprise a needle cover positioning structure. The needle cover positioning structure may comprise at least one needle cover front stop 724. The needle cover front stop 724 may comprise at least one protrusion protruding radially inwardly from the inner surface of the sidewall 700a of the device body 700. The needle cover front stop 724 may comprise a ramp-like section 724.1 with a radial inward inclination in the proximal direction P and a cuboid section 724.2 (see FIG. 7F). The cuboid section 724.2 may be arranged proximal to the ramp-like section 724.1. The cuboid section 724.2 may protrude in a radial inward direction as much as a proximal end of the ramp-like section 724.1. Alternatively, the cuboid section 724.2 may protrude in a radial inward direction more or less than the ramp-like section 724.1.

The needle cover front stop 724 may be configured to interact with a corresponding securing feature of the needle cover 500. The securing feature may be the front stop slot 505 of the needle cover 500. For example, a distal facing surface of the front stop slot 505 may abut on a proximal facing surface of the cuboid section 724.2, when the needle cover 500 is moved distally relative to the device body 700, e.g. from the second cover position Y to the third cover position Z, as described above. Thus, the needle cover front stop 724 is configured to provide a limitation for a distal movement of the needle cover 500 relative to the device body 700. In order words, a maximum distal extension of the needle cover 500 beyond the distal end of the device body 700, is defined by the interaction between the front stop slot 505 and the needle cover front stop 724, e.g. after use, when the needle cover 500 is in its final position relative to the device body 700.

The ramp-like section 724.1 may be configured to deflect the side regions 503 of the needle cover 500 radially inwardly when the needle cover 500 is inserted into the device body 700, thereby enabling an assembly of the needle cover 500 in the device body 700. The cuboid section 724.2 may provide additional axial strength to the needle cover front stop 724, thereby improving the stability of the device body 700. For example, this may be beneficial for withstanding the distal force applied to the needle cover 500 by the needle cover spring 600.

In one embodiment, the device body 700 may comprise a needle cover back stop 721. The needle cover back stop 721 may be formed at a distal end of at least one connecting rib 708. As illustrated in FIG. 7C, the needle cover back stop 721 may be arranged at the outer section of the connecting rib 708, where the connecting rib 708 is connected to the inner surface of the sidewall 700a. The needle cover back stop 721 comprises a distal surface which may extend to a distal end of the axial support front end 703. The distal surface may interact with the needle cover 500, e.g. with a proximal facing surface of a recess between the side region 503 and the flexible arm 510, when the needle cover 500 moves proximally relative to the device body 700, e.g. when the skin contact surface 501 is pressed against the user's skin with a sufficient force, as described above. Thus, the needle cover back stop 721 may define the maximum proximal position of the needle cover 500 relative to the device body 700.

The embodiment illustrated in FIGS. 7C and 7F comprises four connecting ribs 708, each of which having a needle cover backstop 721, as described before. The needle cover back stop 721 extends from the connecting rib 708 in the distal direction beyond needle cover spring support 708a, e.g. up to the protrusion 704 (see also FIG. 6B). Alternatively, the needle cover back stop 721 may extend farther or less far in the distal direction.

In one embodiment, the device body 700 may comprise a needle cover lock structure 720. The needle cover lock structure 720 may comprise at least one protrusion, e.g. one or more ramp-like elements 720 (see FIGS. 7B, 7C, and 7F), protruding radially inwardly from an inner surface of the sidewall 700a of the device body 700. The ramp-like elements 720 may have a radial inward inclination in the distal direction D.

The needle cover lock structure 720 may be arranged close to the distal end of the device body 700, e.g. in the last 30 percent, in the last 20 percent or in the last 10 percent of the axial length of the device body 700, when measured from the edge 732 to the distal end. The needle cover lock structure 720 may be aligned with the drug window 710 in the circumferential direction. The needle cover lock structure 720 may be distal to the drug window 710. The needle cover lock structure 720 may interact with the flexible arms 510 on the needle cover 500 (as described above and below). Due to this interaction, a proximal movement of the needle cover 500 relative to the device body 700 may be limited, for example, avoided, when the needle cover 500 is in a distal position relative to the device body 700, e.g. after injection. In other words, the interaction may provide an end of dose lockout function.

In one embodiment illustrated in FIGS. 7B, 7C and 7F, the needle cover lock structure 720 may comprise at least one ramp-like element 720. The ramp-like element 720 may be offset from the needle cover front stop 724 in the axial direction. For example, the ramp-like element 720 may be distally offset from the needle cover front stop 724.

Due to the radial inward inclination in the distal direction D of the ramp-like elements 720, when the needle cover 500 moves distally relative to the device body 700 and the flexible arms 510 are proximal to the ramp-like element 720, the flexible arms 510 deflect radially inwardly. After having passed the ramp-like elements 720, the flexible arms 510 may return to their relaxed state by deflecting radially outwardly. Thus, a proximal surface of the cuboidal elevation 510.1 interacts with a distal surface 720a of the ramp-like element 720. For example, the distal surface 720a of the ramp-like element 720 may by perpendicular to the axial direction or only slightly inclined thereto, such that when a force in the proximal direction P is applied to the needle cover 500, a sliding of the cuboidal elevation 510.1 along the distal surface is limited. In other words, the needle cover 500 is locked against a proximal movement relative to the device body 700 by the interaction of its cuboidal elevation 510.1 with the distal surface of the ramp-like element 720.

In one embodiment as illustrated in FIG. 7G, the needle cover lock structure 720 comprises four ramp-like elements 720, grouped in two pairs. In other words, the needle cover lock structure 720 comprises two double-ramps, consisting of two ramp-like elements 720 each. The ramp-like elements 720 may be distally offset from the drug window 710 (see FIG. 7F). Each pair of ramp-like elements 720 may be aligned with the drug window 710 in the circumferential direction. The two ramp-like elements 720 of a pair may be arranged such that an angle between them is less than 90 degrees, for example, less than 45 degrees in a circumferential direction. A space may be formed between the two ramp-like elements of a pair. In the circumferential direction, each pair may be aligned with the flexible arms 510 of the needle cover 500 such that, when the needle cover 500 moves distally relative to the device body 700, the web 510.2 of the needle cover 500 is guided between the two ramp-like elements (as described above). Further, due to the web 510.2 extending into the space between the ramp-like elements 720, there is less radial inward deflection of the flexible arms 510 when sliding along the ramp-like elements 720.

The two pairs of ramp-like elements 720 may be angularly offset from each other, e.g. by 180 degrees. Further, the two pairs may be angularly offset from the needle cover front stops 724, e.g. by 90 degrees.

After the cuboidal elevation 510.1 has passed a distal end of the ramp-like element 720, the flexible arms 510 deflect radially outwardly. Thus, a proximal surface of the cuboidal elevation 510.1 interacts with a distal surface of the ramp-like elements 720, when a force in a proximal direction is applied to the needle cover 500 after use (see FIG. 7G). Due to the interaction, the needle cover 500 is prevented from moving proximally relative to the device body 700, i.e. the after-use needle cover lock mechanism.

In the embodiment illustrated in FIGS. 7B, 7C, 7F and 7G, the device body 700 comprises two needle cover front stops 724 and the needle cover lock structure 720 is formed by four ramp-like elements 720. However, there can be more or less than two needle cover front stops 724 and more or less four ramp-like elements 720. The needle cover front stops 724 may be angularly offset from the ramp-like elements 720, e.g. by 90 degrees or more or less, depending on geometry of the needle cover 500 and/or the number ramp-like elements 720 and/or the number of front stops 724. The ramp-like elements 720 may be grouped, e.g. in pairs, such that the pairs of ramp-like elements 720 are angularly offset from each in the circumferential direction, for example, such that the pairs are arranged equidistantly in the circumferential direction. For example, the angle between two pairs may be 180 degrees. Each pair of ramp-like elements 720 may be offset from each needle cover front stop 724 by 90 degrees in the circumferential direction. Thus, the pairs of ramp-like elements 720 and the needle cover front stops 724 may be arranged equidistantly around the circumference of the inner surface of the sidewall 700a. For example, there may be two pairs of ramp-like structures 720, which are angularly offset from the needle cover front stops 724 by 90 degrees, while the front stops are angularly offset from each other by 180 degrees. The needle cover front stops 724 may be arranged proximally offset from the ramp-like elements 720.

In one embodiment, the needle cover front stops 724 may be arranged with an angular offset of 90 degrees with respect to the drug window 710. The needle cover front stops 724 may be, at least partially, overlapped by the drug window 710 in the axial direction (see FIG. 7D).

In one embodiment, the device body 700 may comprise a needle cover lock disengagement prevention structure 720.1. The needle cover lock disengagement prevention structure 720.1 may comprise a protrusion, e.g. a rib 720.1, protruding radially inwardly from the inner surface of the sidewall 700a and extending in the longitudinal direction of the device body 700 (see FIG. 7C). The needle cover lock disengagement prevention structure 720.1 is configured to limit a deformation of the needle cover 500 relative to the device body 700, when the needle cover 500 is in interaction with the needle cover lock structure 720 and/or the needle cover front stop 724. This may limit the risk of disengagement of the needle cover 500, e.g., the cuboidal elevation 510.2 of the flexible arms 510 or the slot 505, from the needle cover lock structure 720 or the front stop 724, when the device body 700 deforms relative to the needle cover 500, e.g. because the body is dropped or squeezed be the user. Additionally, the needle cover lock disengagement prevention structure 720.1 may provide stiffness to the sidewall 700a.

As illustrated in FIGS. 7C and 7F, the needle cover lock disengagement prevention structure 720.1 may comprise eight elements, e.g. eight ribs 720.1, arranged with different angular offsets at the sidewall 700a. The ribs 720.1 may be arranged such that a pair of ribs 720.1 may be angularly offset from two single ribs 720.1. For example, a pair of ribs 720.1 may be enclosed by two single ribs 720.1 in the circumferential direction in each half of the circumference of the sidewall 700a. The ribs 720.1 may extend from a proximal end of the ramp-like elements 720 in the proximal direction. In one embodiment not illustrated, the ribs 720.1 may overlap the ramp-like structures 720 and/or the front stops 724 in the axial direction.

In the proximal direction, the ribs 720.1 may have a decreasing radial inward extension from the sidewall 700a. In one embodiment, the ribs 720.1 may transition to the inner surface of the sidewall 700a, e.g. in a portion overlapping with the central support window 709 in the axial direction. For example, the radial inward extension from the sidewall 700a may become zero at an axial position corresponding to a proximal end of the central support window 709 or a proximal end of the needle cover front stop 724.

In one embodiment, the device body 700 may comprise at least one needle cover guide rib 723 (see FIG. 7C). The needle cover guide rib 723 may be configured to prevent a rotation of the needle cover 500 relative to the device body 700. For example, the needle cover guide rib 723 may interact with a side region 503 of the needle cover, e.g. with the lateral edge 503.1 of the side region 503, thereby preventing a rotational movement of the needle cover 500 relative to the device body 700. The needle cover guide rib 723 may be formed on a circumferentially facing surface of at least one of the connecting ribs 708, several of the connecting ribs 708 or all of the connecting ribs 708. The needle cover guide rib 723 may extend in the axial direction of the central support structure 701. For example, the needle cover guide rib 723 may extend axially from a proximal end of the axial support front end 703 up to a distal end of a holder guide rib 726 (described below). Other axial extensions, e.g. with reference to the central support window 709 are possible, as well, as long as the needle cover guide rib 723 is arranged to interact with and provide a rotational support to the needle cover along the full axial movement of the needle cover 500 relative to the device body 700. The needle cover guide rib 723 may have a triangular cross-section, such that, a surface of the needle cover guide rib 723 extending predominantly in the circumferential direction may be shorter than a surface of the needle cover guide rib extending predominantly in the radial direction. Thus, a larger interaction surface for interacting with the lateral edge 503.1 of the side region 503 may be formed. This may beneficial for preventing a disengagement of the lateral edge 503.1 from the needle cover guide rib 723.

In one embodiment, the needle cover guide rib 723 is formed on a circumferential surface (lateral) of the connecting rib 708, e.g. a circumferential surface facing towards a needle cover radial support rib 722 (described below) and/or facing towards one of the ribs 720.1 of the needle cover lock disengagement prevention structure.

In one embodiment, the device body 700 may further comprise at least one needle cover radial support rib 722 (see FIG. 7C). The needle cover radial support rib 722 may be configured to support the side regions 503 of the needle cover 500 such that the side regions 503 are prevented from deflecting radially inwardly. In some examples, this may be relevant when the device body 700 is deformed, e.g. squeezed. In this case, the part of the needle cover 500 extending inside the device body 700 may be deformed as well. This might cause a disengagement of the lateral edges 503.1 from the needle cover guide ribs 723 or a disengagement of the cuboidal elevation 510.1 from the needle cover lock structure 720, which both are potentially dangerous to the user or the correct function of the drug delivery device.

As illustrated, the needle cover radial support rib 722 may extend radially outwardly from the central support structure 701. In the axial direction, the needle cover radial support rib 722 may extend along the same length as the needle cover guide rib 723. In the axial direction, the needle cover radial support rib 722 may, at least partially, overlap with the needle cover guide rib 723. For example, the needle cover radial support rib 722 and the needle cover guide rib 723 overlap over the full axial length of the shorter of the two ribs. As illustrated in FIG. 7C, the device body 700 may comprise four needle cover radial support ribs 722, e.g. one for each connecting rib 708. In the circumferential direction, the needle cover radial support ribs 722 may be arranged such that they support the side regions 503 of the needle cover 500 along their circumferential extension. For example, in the circumferential direction, the needle cover radial support ribs 722 may be arranged such that the most lateral sections of the side regions 503 of the needle cover 500 are supported against radial inward deflection.

In one embodiment, the needle cover radial support ribs 722 may be arranged to circumferentially overlap the needle cover lock disengagement prevention structure, e.g. the ribs 720.1. Alternatively, the angular offset between the needle cover radial support ribs 722 and the needle cover lock disengagement prevention ribs 720.1 may be small, e.g. less than 45 degrees, less than 20 degrees, less than 10 degrees or less than 5 degrees. A small offset may be advantageous as it may improve the support of the needle cover 500, in both radial directions. In other words, due to the needle cover radial support ribs 722 extending in a radial inward direction and the needle cover lock disengagement prevention ribs 720.1 extending in a radial outward direction, the side regions 503 may be secured against a radial movement.

In one embodiment, the device body 700 may further comprise a syringe holder front stop 705 (see FIGS. 7D and 7E). The syringe holder front stop 705 may be formed in a distal half of the central tube 702, e.g. at a proximal end of the axial support front end 703. The syringe holder front stop 705 may be formed by a proximal facing surface of the central support structure 701. For example, the syringe holder front stop 705 may be formed by a distal end of an axial recess in the central tube 702. The syringe holder front stop 705 may define a final distal position of the syringe holder 800 within the device body 700. For example, the syringe holder front stop 705 may interact with a stop feature 809 (described below) of the syringe holder 800, when the syringe holder 800 is moved distally relative to the device body 700, e.g. during assembly of the syringe holder 800 in the device body 700. As illustrated in FIG. 7E, an axial centerline of the syringe holder front stop 705, which is parallel to the axial direction of the central support structure 701, may be offset by 90 degrees from an axial centerline of the central support window 709 and/or the drug window 710.

In one embodiment, the device body 700 may comprise at least one cap groove 725. The cap groove 725 may be formed on the inner surface of the sidewall 700a. The cap groove 725 may extend axially from the distal end of the device body 700 in the proximal direction P. In the circumferential direction, the extension of the cap groove 725 may cover at least 1 percent of the circumference. The cap groove 725 may be configured to interact with the anti-rotation ribs 205 of the cap 200, thereby preventing a rotation of the cap 200 relative to the device body 700, when the cap 200 is connected to the device body 700, as described above. As illustrated in FIGS. 7B and 7C, the device body 700 may comprise at least four cap grooves 725. The cap grooves 725 may be arranged equidistantly around the circumference of the inner surface of the sidewall 700a. Alternatively, the cap grooves 725 may be arranged with different angular offsets in the circumferential direction.

In one embodiment, the device body 700 may comprise at least one holder guide rib 726 (see FIG. 7B). In one embodiment, the device body 700 comprises four holder guide ribs 726. The holder guide ribs 726 may extend radially inwardly from the sidewall 700a. The holder guide ribs 726 may distally extend from the proximal end of the device body 700, e.g. the aperture 730, until approximately half the length of the central support structure 701. For example, the holder guide ribs 726 may distally extend from the proximal end of the device body 700 up to a proximal end of the needle cover guide ribs 723 and/or up to a proximal end of the needle cover radial support ribs 722. Alternatively, or additionally, the holder guide ribs 726 may distally extend from the proximal end of the device body 700 up to at least a proximal end of the needle cover lock disengagement prevention structure 720.1, e.g. until the axial position where the radial inward extension of the rib 720.1 from the sidewall 700a becomes zero.

The holder guide ribs 726 may have a triangular cross-section or a cross section comprising a rectangle, e.g. a square, with a triangle on its radial inner surface, such that a tip of the triangle points towards a symmetry axis of the device body 700. The holder guide ribs 726 may be angularly offset from each other by at least 30 degrees, at least 45 degrees or at least 60 degrees. In other words, the offset of one holder guide rib 726 to its first neighboring holder guide rib may be smaller than the offset to its second neighboring holder guide rib. In one embodiment, the holder guide ribs 726 may be equidistantly arranged in the circumferential direction.

In one embodiment, the holder guide ribs 726 may be configured to interact with the syringe holder 800. For example, the holder guide ribs 726 may be configured to interact with guiding features 811 of the syringe holder 800 during and/or after the assembly of the syringe holder 800, as described below. For example, the holder guide ribs 726 may prevent the syringe holder 800 from rotating relative to the device body 700, thereby defining the spatial orientation the syringe holder 800 relative to the device body 700, e.g. during and/or after the assembly of the syringe holder 800 to the device body 700.

Alternatively, or additionally, the holder guide ribs 726 may be configured to interact with the drive spring holder 1200 (described below). For example, the holder guide ribs 726 may be configured to interact with guide ribs 1202.1 of the drive spring holder 1200 during and/or after the assembly of the drive spring holder 1200 to the device body 700, as described in more detail in section 12 below. For example, the holder guide ribs 726 may prevent the drive spring holder 1200 from rotating relative to the device body 700, thereby defining the spatial orientation the drive spring holder 1200 relative to the device body 700, e.g. during and/or after the assembly of the drive spring holder 1200 to the device body 700

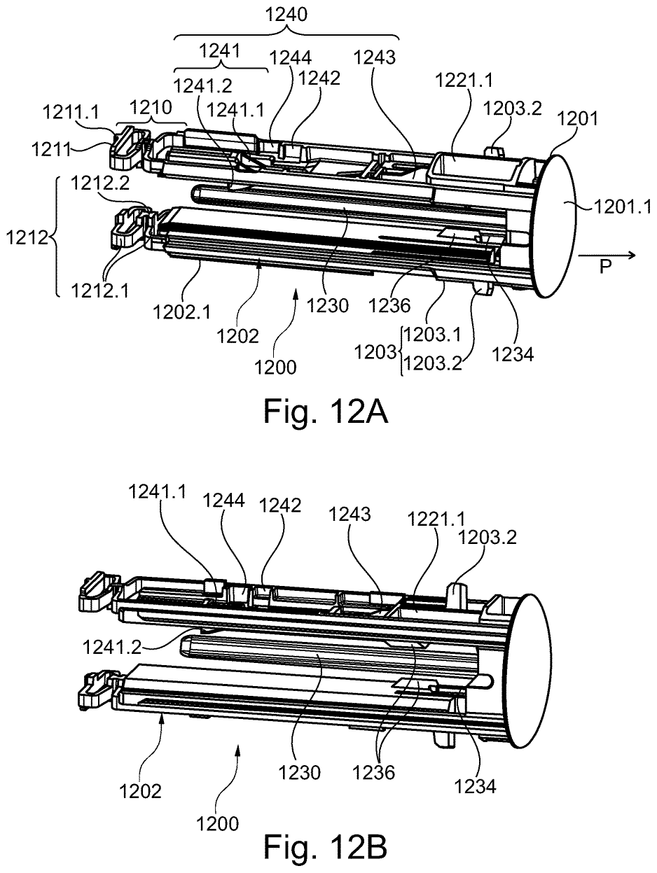

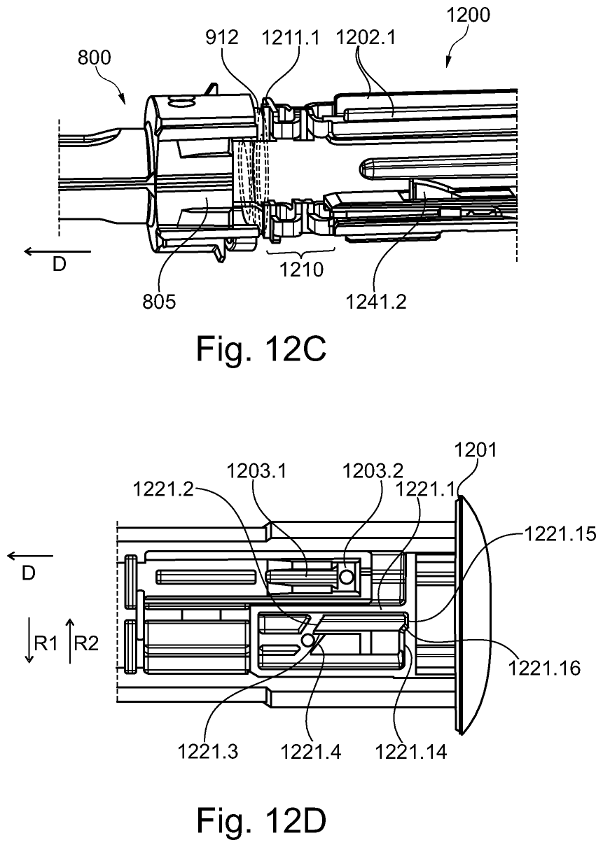

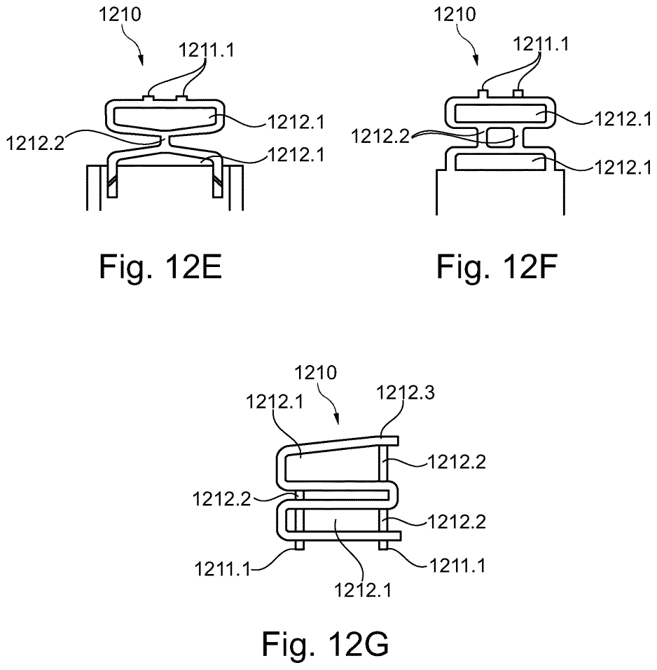

In one embodiment, the device body 700 may comprise at least one, for example, at least four cap ribs 727 (see FIG. 7F). The cap ribs 727 may protrude radially inwardly from the inner surface of the sidewall 700a. The cap ribs 727 are configured to interact with the cap clips 204. For example, the cap ribs 727 may prevent a radial outward movement of the cap clips 204, thereby preventing a disengagement of the cap clips 204 from the cap clip windows 504 of the needle cover 500. As illustrated in FIG. 12F, the device body 700 may comprise two groups having three cap ribs 727 each. The groups may be angularly offset from each other by 180 degrees.

The cap ribs 727 within each group may be equidistantly arranged in the circumferential direction. Each group of caps ribs 727 may be formed at substantially the same axial position as the needle cover locking elements 720 with an angular offset of 90 degrees thereto. In other words, the group of cap ribs 727 may be aligned with the needle cover fronts stops 724 in the circumferential direction. The cap ribs 727 may be arranged distally of the needle cover front stop 724.

In one embodiment, the device body 700 comprises a label on an outer surface of the sidewall 700a. The label may be attached to or connected to or directly integrated in the sidewall 700a. The label may prevent the injection molding gate recess 712 and/or the cut-outs 713, 714 from being viewable by the user. This may provide comfort to the user. The label may contain information regarding the drug delivery device 100, e.g. information about the drug Dr to be administered with the drug delivery device 100 or a date of manufacturing of the drug delivery device 100.

In one embodiment, the label comprises an NFC (Near Field Communication) tag. The NFC tag may be a passive NFC tag, e.g. configured to direct the user to a website or an app. Alternatively, or additionally, the NFC tag may be an active NFC tag which may function as a sensor. For example, the NFC tag may be an RFID (Radio Frequency Identification) tag.

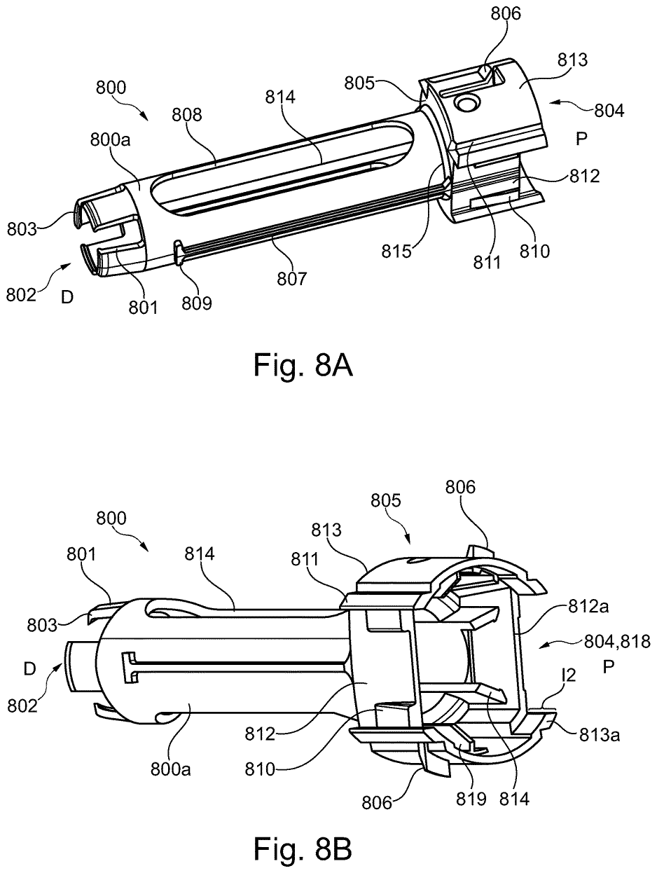

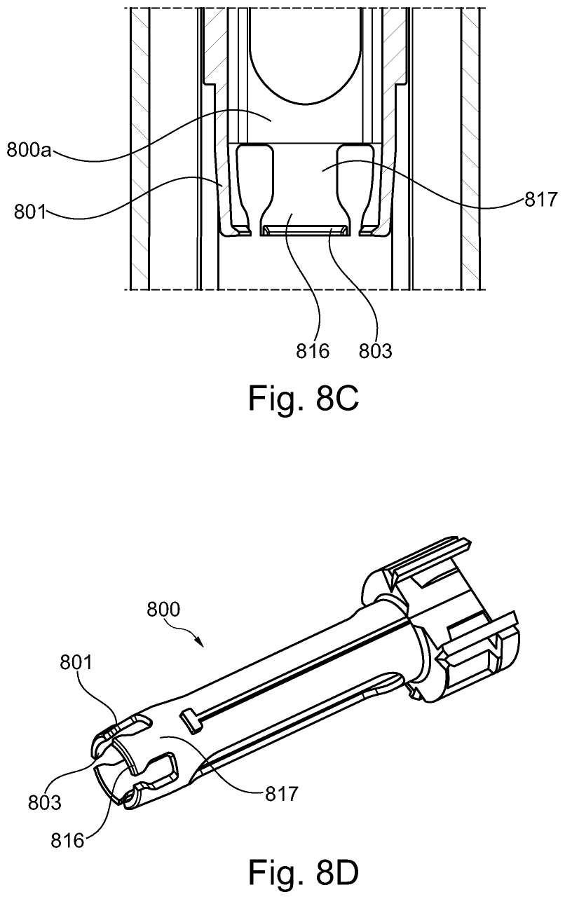

### 8. Syringe Holder (FIG. 8A to 8D)

FIGS. 8A and 8B illustrate an optional syringe holder 800 to allow an accurate support of the pre-filled syringe 900 during and after assembling. The drug delivery device may comprise the syringe holder 800, e.g. a container holder, in specific embodiments. The syringe holder 800 may be adapted to assemble and hold the pre-filled syringe 900, e.g. a medicament container, within the device body 700 and is further explained in more detail in the following.

For example, the syringe 900 may be a 1.0 ml pre-filled syringe 900 with a rigid protective needle shield 914 (RNS). Usually, the syringe 900 and/or the protective needle shield 914 (also called “needle shield”) may have variations in dimensions, e.g. in length and/or in diameter. To allow accurate support of the pre-filled syringe 900 in a mounted position despite these variations, the design of the syringe holder 800 and the device body 700 (front case) may be adapted to displace and position the needle shield 914 to a predetermined position during assembly to provide sufficient clearance to support the pre-filled syringe 900 at its datum in the mounted position. The datum may be a distal shoulder of the syringe barrel. Alternatively or additionally, the radial diameter of the syringe shoulder may be less than the one of the needle shield, e.g. often the case for 1 ml syringes, such that the syringe holder facilitates providing access to the shoulder of the syringe.

Therefore, the syringe holder 800 may comprise flexible holder arms 801 adapted to engage and/or position the syringe 900 and/or hold it in a mounted position. The flexible holder arms 801 may protrude inwards in a relaxed state. Alternatively, the flexible holder arms 801 may be protruded outwards in a relaxed state. Other configurations of the arms in the relaxed state are also possible.

The syringe holder 800 may comprise a holder housing 800a, e.g. a main body portion, adapted to receive the pre-filled syringe 900 and at least two flexible holder arms 801 adapted to couple with the pre-filled syringe 900 in the mounted position, e.g. four flexible holder arms 801. The holder housing 800a may be formed as a hollow cylinder or cylindrical portion.

The flexible holder arms 801 may be distally extended from an axial holder front end 802, e.g. a distal end 802, of the holder housing 800a and may be protruded inwards in a relaxed state, e.g. are inwardly formed, e.g. angled. The flexible holder arms 801 may comprise at its distal ends holder protrusions 803 that may be directed radially, e.g. inwardly.

The flexible holder arms 801 may have the same width throughout their extension. i.e. the wideness of a one flexible holder arm at the syringe holder front end 802 corresponds to the wideness of the flexible holder arm at its distal end (see FIG. 8A).

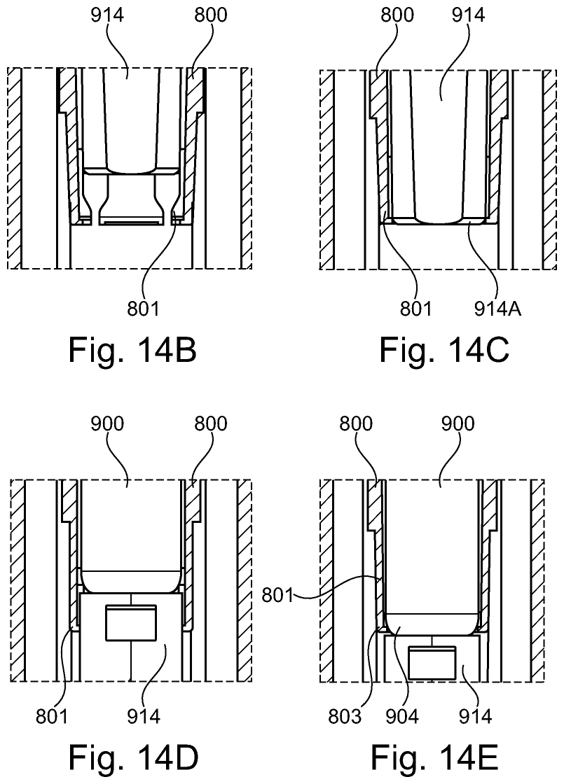

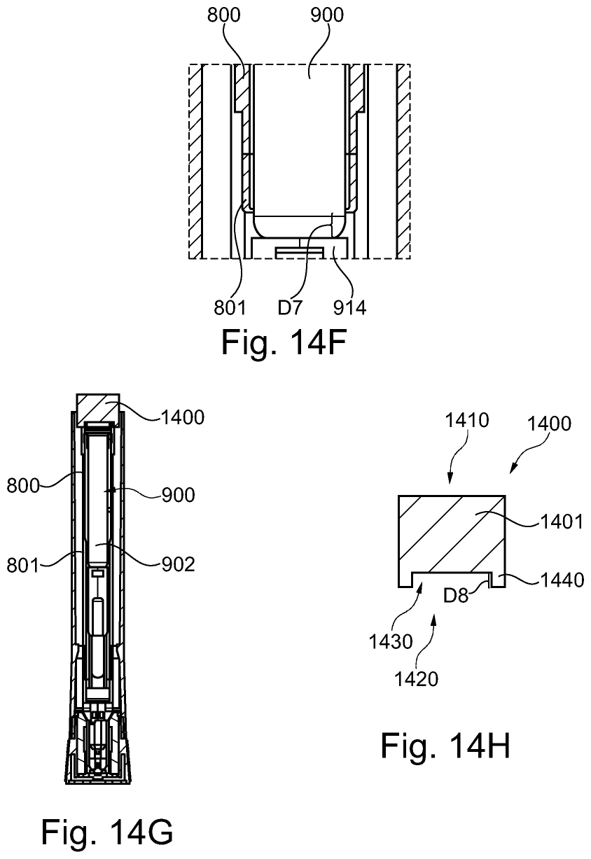

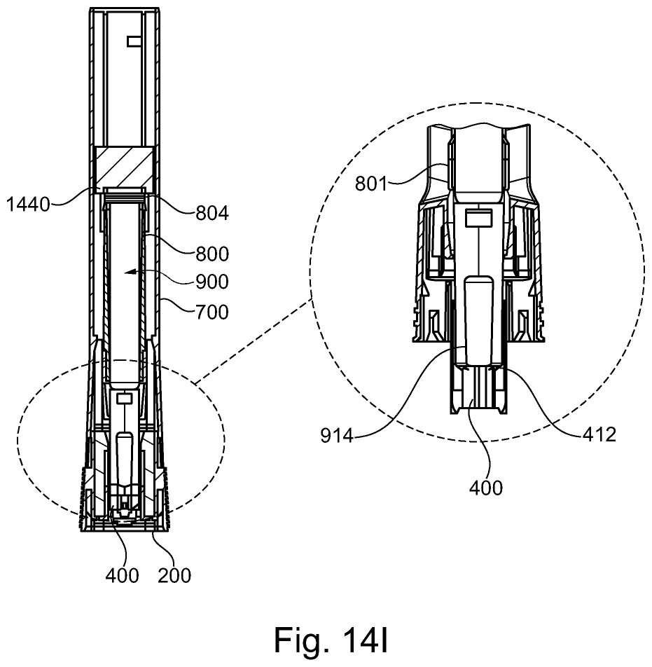

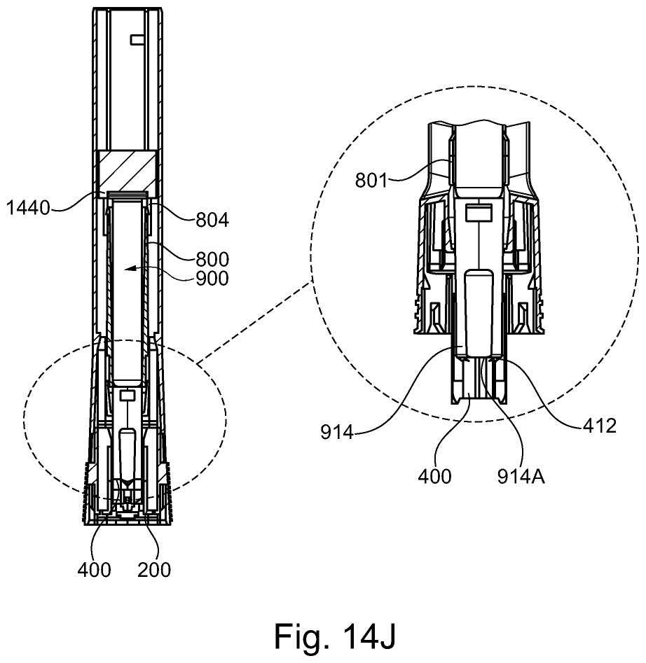

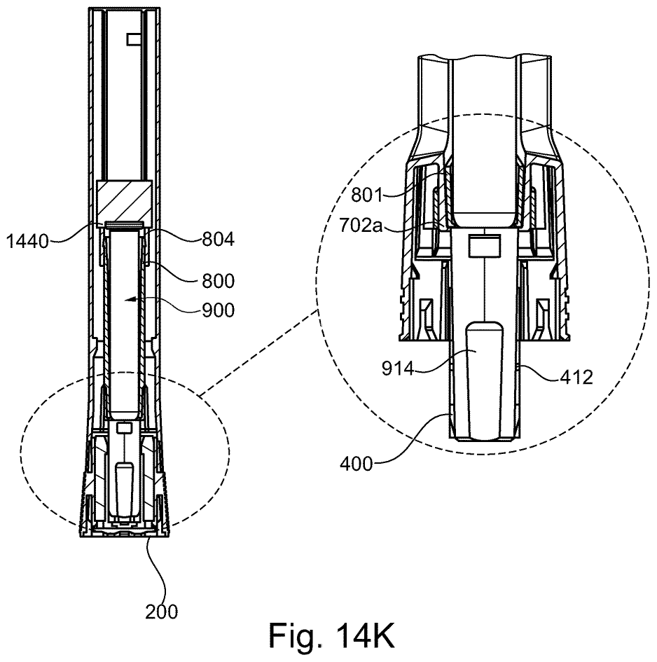

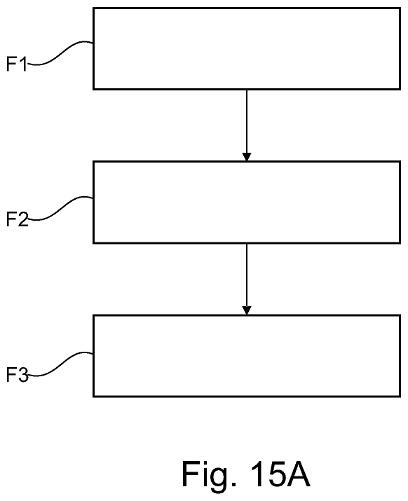

The holder protrusions 803 may comprise on their radially inner facing surface ramps increasing in height in a proximal direction (best seen in FIG. 8B) which aid the assembly process as described below in FIGS. 14B to 14E.

The holder protrusions 803 may have the function of stabilizing the connection between the syringe holder 800 and the pre-filled syringe 900 in a mounted position of the pre-filled syringe 900 and/or of the syringe holder. In some examples, the holder protrusion 813 may be adapted to engage the space between the proximal end of the needle shield 914 and the shoulder of the pre-filed syringe 900.

To support the final assembling of the pre-filled syringe 900 into the syringe holder 800, the at least two flexible holder arms 801 may be adapted to couple with the pre-filled syringe 900 in the mounted position in such a manner that the outwardly pre-stressed flexible holder arms 801 return back or snap back radially inwards, e.g. into the relaxed state, in the mounted position, e.g. between the rigid needle shield 914 and the shoulder 904 of the pre-filled syringe 900. The flexible holder arms 801 may return back into the relaxed state due to a relative movement of the syringe holder 800 with respect to the syringe 900, e.g. a distal movement of the syringe holder. This relative movement may be caused by an axial force operating on the syringe holder 800, e.g. on a holder rear end 804.

Furthermore, the device body 700 (front case) may be adapted to restrain the outward deflection of the flexible holder arms 801 when the syringe 900 is in the mounted position. This secured the syringe in the syringe holder.

The syringe holder 800 may comprise a holder rear end 804, i.e. proximal end, opposite to the holder front end 802. At the holder rear end 804, the holder 800 may comprise a holder flange portion 805 that comprises holding clips 806 for releasable intermittent holding of the syringe holder 800 relative to the device body 700.

The holder flange portion 805 may form a receiving space 818 through which a pre-filled syringe 900 is inserted distally into the hollow cylinder formed by the holder body 800a.

The holder flange portion 805 may be non-circular, e.g. comprising two rounded sections 813 of the holder flange portion 805 arranged oppositely, e.g. diametrically opposite, to each other and two in a radial inward direction recessed sections 812 being flatter than the rounded sections 813. As such the two rounded sections extend circumferentially as semicircles. The two recessed sections may be arranged at the other opposite, e.g. diametrically opposite, ends of the holder flange portion 805 and on opposite sides of the holder flange portion 805 with respect to the rounded sections 813. The two recessed sections 812 may define a recessed outer flange surface.

The two rounded sections 813 may be circumferentially aligned with the windows 814 of the syringe holder 800. This may be because the cut out 714 as described with respect to the body 700 may be circumferentially aligned with the drug window 710 of the device body 700. The recessed sections 812 may be arranged circumferentially 90 degrees offset from the windows 814 of the syringe holder 800 and may be circumferentially aligned to the ribs 807, described later in more detail.

The axial extension of the recessed sections 812 in the proximal direction may be less than the axial extension of the rounded sections 813 in proximal directions, as indicated by the length 12 in FIG. 8B. In other words, the proximal end 813a of the rounded sections 813 may be further proximal than the proximal end 812a of the recessed sections 812.

As such the two rounded sections 813 extending axially in proximal direction further out, may define a receiving space for receiving the syringe flange of the pre-filled syringe 900 when mounted on the syringe holder 800.

Once mounted the pre-filled syringe 900 may be rotationally locked with respect to the syringe holder 800 and/or with respect to the device body 700. Specifically, the rounded sections 813 may comprise on the inner surface ribs 819, which may prevent the rotation of the mounted pre-filled syringe 900 in an assembled state.

The pre-filled syringe 900 may however also be rotatable with respect to the syringe holder 800 and/or the device body 700 in embodiments where such a rotation is deemed necessary or advantageous.

The two recessed sections 812 may be recesses 812 in the holder flange portion 805 and extending axially throughout the entire holder flange portion 805. The edges between the rounded sections 813 of the holder flange portion 805 and the flat recesses of the holder flange portion 805 may also comprise or form guiding features 811, for aiding the positioning of the syringe holder 800 into the body during assembly. In other words the syringe holder 800 may comprise guiding features 811 extending along the longitudinal axis on the holder flange portion 805, the guiding features 811 being arranged on the sidewalls of the holder flange portion 805 delimiting the space defined by the recessed sections 812.

The flat recessed sections may form a receiving space for the needle cover arms, e.g. during the assembly process or in the device.

Each recess may further comprise at least one, e.g. two ramp-like protrusion 810 increasing in height towards the syringe holder rear end 804, e.g. proximal end 804. The ramp-like protrusion 810 may serve as guiding features for the needle cover arms during the assembly step of inserting the pre-filled syringe 900 and the syringe holder 800 into the device body 700.

The ramp-like protrusion 810 may be arranged proximally on the recessed section 812 such hat the proximal end of the ramp-like protrusions 810 lie substantially in a plane with the proximal end 812a of the recessed section 812.

The two ramp-like protrusions 810 may be arranged angularly in opposite ends of the recessed outer flange surface. In case of two ramp-like protrusions, the two ramp-like protrusions may define a channel arranged centrally between the two-ramp-like protrusions, the channel being configured to permit the passing of a rib of a portion of the needle cover during assembly of the drug delivery device and/or in a mounted position of the container holder.

The recessed section 812 may be radially delimited by a surface of the holder flange portion 805. The holder flange portion 805 may be a rounded section 813 of the proximal end region of the holder body. The recessed section 812 may define a space delimited angularly by sidewalls of the holder flange portion 805, wherein the recessed section 812 may be open on a distal end and on a proximal end.

The space defined by the recessed section 812 may be adapted to engage and/or receive a portion of a needle cover of a drug delivery device in a mounted position of the container holder 800. The space defined by the recessed section 812 has an inwardly radial depth which decreases in the proximal direction. In some examples, this is useful when assembling the drug delivery device 100 as it deflects the needle cover side regions, e.g. legs, as required during assembly of the drug delivery device. The space defined by the recessed section 812 has an inwardly radial depth which does not change.

The holding clips 806 may be integrally formed on the holder flange portion 805 as tongues or clips. For example, the holding clips 806 may have a flexible portion, which substantially extends in the axial direction and which is deflectable in the radial direction. The holding clips 806 may be arranged on the rounded sections 813 of the holder flange portion 805. The holding clips 806 may be arranged circumferentially offset with respect to a middle point of the respective rounded section 813 on which they are arranged. The holding clips 806 may be on circumferential opposite sides to each other.

The holding clips 806, e.g., the flexible portion thereof, may further extended axially from a distal end of the holder flange portion 805 towards the proximal end of the holder flange portion 805. The holding clips 806 may be configured to not extend throughout the axial elongation of the holder flange portion 805. In some examples, the holding clips 806 may be extended in a proximal direction for the same length as the retracted portions, e.g. the recess portions 812.

In other words the holding clip 806 may extend proximally less than the holder flange portion 805.

Proximal ends of the holding clips 806 may be radially outwardly directed to engage cut-outs 713, 714 of device body 700. In one embodiment, the proximal ends may have an inclined surface with a radial outward inclination in the proximal direction P. In an embodiment, the syringe holder 800 may comprise two holding clips 806 arranged opposite to each other. Instead of cut-outs, the device body 700 may comprise an inner support to releasable hold the holding clips 806. For example, the inner support may be formed as an inner groove.

The holding clips 806 are configured such that in a first engaged position of the syringe holder, the holding clamps 806 interact with slots, e.g. the proximal cut-outs 714 of the device body 700. In the first engaged position, the syringe holder 800 may be moved distally, but may be prevented from moving proximally relative to the device body 700, thereby exiting the device body 700. For example, this can be achieved for example through a ramped distal surface of the holding clips 806, e.g. a ramp which increases in height in its proximal direction. Further, in the first engaged position, the syringe holder 800 may be prevented from rotating relative to the device body 700.

The holding clips 806 are configured such that, when the syringe holder 800 is moved distally, e.g. during its assembly process, the holding clips 806 disengage from the cut-outs 714. A further distal movement of the syringe holder 800 re-biases the holding clips 806 radially inwards until the holding clips 806 align with distal slots, e.g. the distal cut-outs 713 of the device body 700. Upon alignment, the holding clips 806 interact with the cut-outs 713 by deflecting radially outwardly into the space formed by the cut-outs 713. This is the second engaged position of the syringe holder 800. The interaction between the holding clips 806 and the cut-outs 713 prevents a proximal movement as well as a rotational movement of the syringe holder 800 relative to the device body 700.

At least one optional longitudinal rib 807 may be arranged on the holder housing 800a, e.g. two ribs on sides that are opposite to each other. The longitudinal rib 807 may be used to position the syringe holder 800 axially with regard to device body 700. The longitudinal rib 807 may comprise a stop feature 809 positioned at the end of longitudinal rib 807 towards the holder front end 802. The stop feature 809 is configured to abut a respective element of the device body as shown and described with regards to FIG. 7E. This may limit distal movement of the syringe holder relative to the device body. The stop feature may be wider than the longitudinal rib 807.

Moreover, the holder housing 800a may comprise at least one elongated holder window 808 in order to enable visual inspection of the amount of drug Dr within pre-filled syringe 900 when syringe 900 is mounted within syringe holder 800. The holder housing 800a may comprise the two elongated holder windows 808 on sides that are opposite to each other.

The holder windows 808 may be configured such as to be bigger in size than the drug window 710 of the body 700, such as to reduce the visibility, e.g. hide, the holder housing 800a when looking through the drug window 710 of the body 700. This increases the confidence of users, e.g. patents, as they are not confronted with any internal parts off the drug delivery device and are provided with an unobstructed view of the pre-filled syringe 900 through the holder windows 808 and the drug window 710 of the body 700.

The inner surface of the holder housing 800a may further comprise longitudinal ribs, e.g. support ribs 814 which extend substantially along the inner surface of the holder housing 800a. The support robs 814 may extend further proximal than the holder housing 800a, such as to extend on the inner surface of the recessed sections 812.

The support ribs 804 may serve as support for the syringe barrel 902, once the pre-filled syringe 900 is inserted into the syringe holder 800. The support ribs 814 further have the function of centralizing the pre-filled syringe 900 once inserted into the syringe holder 800, guaranteeing thereby that the pre-filled syringe 900 lies central insider the holder body 800a. Through the centralization of the pre-filled syringe 900 by the supporting ribs 804, the needle of the pre-filled syringe 900 lies on axially parallel with respect to the axial extension of the drug delivery device and, for example, central with respect to the circumference defined by the body of drug delivery device. This guarantees a more precise injection process.

FIG. 7D illustrates the arrangement of the syringe holder 800 within device body 700. The essentially cylindrical central syringe support 701, may be shorter than half of the length of the barrel 902 of syringe 900 or shorter than one quarter of length of barrel 902 syringe 900, e.g. in order to save plastic material. Thus, elongated window(s) may not be necessary and may not be present within central syringe support 701.

An outer diameter of needle shield 914 may be essentially equal to an outer diameter of barrel 902 of syringe 900. Thus, the distal ends of flexible holder arms 801, e.g. the radially inwardly extending holder protrusions 803 may be arranged between shoulder 904 and proximal end of needle shield 914. The needle shield 914 may be moved a slight distance into the distal direction D thereby. However, sterility of the needle 908 may still be guaranteed thereby. Removal of the cap 200 and of needle shield 914 may be eased by providing a gap between proximal end of the needle shield 914 and the shoulder 904. In other embodiments, the needle shield may not be moved.

As shown in FIGS. 3A and 7A, to further facilitate handling for the user, especially when removing the cap 200, the device body 700 comprises the body-user-indicator 733 on its outer surface. The body-user-indicator 733 may be a gripping surface. For example, the device body 700 has two body-user-indicators 733, which are arranged opposite each other. The body-user-indicator 733 has the shape of three rectangles located on the distal end of the device body 700, the area size of the three rectangles increasing in the distal direction D and the rectangles adjoining each other in the axial direction A. The cap-user-indicator 203 (as described in section 3 above) and the body-user-indicator 733 may form the user-indicator. The body-user-indicator 733 thus shows the user in which direction the cap 200 must be pulled when removing it from the drug delivery device 100. Since the rectangles are formed as a recess in the device body 700, the rectangles also support the user's secure grip when gripping the device 100. Thus, the body-user-indicator 733 provides both a visual and haptic aid to the user. The body-user-indicator 733 is located distally relative to the drug window along the longitudinal axis of the device body 700. The body-user-indicator 733 can have three rectangular recesses. The side lines of the rectangles running transversely to the longitudinal axis can have the same length and the side lines of the rectangles running along the longitudinal axis can increase in length in the distal direction. Moreover, the recess of the body-user-indicator 733, which is arranged furthest distally, may be positioned directly against an opening of the device body. The cap-user-indicator 203 can have two recesses, wherein the first recess has the shape of an arrow and the second recess has the shape of a rectangle or a trapeze. The recess with the arrow may be located distally from the recess with the trapezoidal or rectangular shape.

FIG. 8C shows an exemplary embodiment of a possible alternative or additional shape of at least one flexible holder arms 801 of a syringe holder 800.

The flexible holder arms 801 in this embodiment may comprise a distal portion 816 configured to have a wider width than a proximal portion 817 of the flexible holder arms 801.

The distal portion 816 of the flexible arms 801 of FIG. 8C is wider than the distal portion 816 of the flexible holder arms 801 of FIG. 8A. In other words, the width of the flexible arms, seen circumferentially, decreases proximally. By having a wider distal portion 816, the holder protrusions 803 directed radially inwardly and arranged on the distal end of the flexible holder arms 801 are also wider, thereby increasing the contact surface of the holder protrusion with the pre-filled syringe 900, e.g. with the barrel 902 of the pre-filled syringe 900 (see e.g. FIGS. 14a to 14K).

This increases the holding stability of the syringe holder 800 for the pre-filled syringe 900. For example, it has been proven to be advantageous, as it provides an additional stability against a movement in the distal direction of the pre-filled syringe caused for example by the impact of the pre-filled syringe 900 on the flexible holder arm 801 during the injection process, e.g. during an initial impact of a spring-driven plunger on a plunger stopper of the syringe.

FIG. 8D shows a further exemplary embodiment of a syringe holder 800 in which the flexible holder arms 801 of FIG. 8C have been implemented on all four flexible holder arms 801 of the syringe holder 800. The other features of the syringe holder 800 of FIG. 8D are analogous to the features in the syringe holder 800 of FIGS. 8A and 8B.

### 9. Pre-Filled Syringe (FIG. 9)

FIG. 9 illustrates the optional pre-filled syringe 900. For example, the syringe 900 may be a 1.0 ml pre-filled syringe 900 with a RNS 914 or a SNS 914 covering the hollow needle 908.

Other volumes of drug Dr are possible as well. Usually, the pre-filled syringe 900 and the needle shield 914 may have variations in dimensions, e.g. in length and/or in diameter. The needle shield 914 may be configured to cover the needle 908 as well as part of a cone 906 of the front end, e.g. the distal end, of the pre-filled syringe 900. The needle shield 914 may be further configured such that in a mounted position a space is left between the proximal end of the needle shield 914 and the shoulder 904 of the pre-filled syringe 900. The pre-filled syringe 900 may further comprise the syringe flange 912 at its proximal end.

The pre-filled syringe 900 further comprises a barrel 902 comprising a drug Dr, e.g., a medicament M to be injected for example to a patient or user.

Before injection begins, the needle shield 914 has to be removed in order to expose the needle 908. This can be achieved through the removal of the cap 200 with the grabber 400 of the drug delivery device 100 as described above with reference to the grabber 400 and the cap 200. During injection the plunger stopper 910 inserted in the barrel 902 may be pushed towards the distal end of the barrel 902, e.g. towards the needle 908, such as to push the drug Dr, e.g. the medicament M, towards the distal end of the pre-filled syringe 900 and out of the needle 908 into the injection area. The plunger stopper 910 may be configured to prevent the drug Dr to exit the barrel 902 from the proximal direction but to be slidable along the barrel 902 when a force is acting on it towards the distal direction, e.g. towards the needle 908.

Examples of pre-filled syringes 900 are the BD (Becton Dickinson) Neopak 1 ml long pre-filled syringe (with a 27 gauge special-thin-walled needle) and the Ompi EZ-Fill 1 ml long pre-filled syringe (with a 27 gauge thin-walled needle). Both these syringes comprise a RNS and a West 2340 Flurotec Plunger Stopper. Other syringes or other medicament containers may be used as well, especially comprising different amounts of drug Dr volume and/or different needle diameters, especially outer diameters.

According to at least one embodiment the dose volume may range between 0.5-1.14 ml (milliliter), and the viscosity of the drug between 1-25 cP (Centipoise).

Further examples of pre-filled syringes may be a BD (Becton Dickinson) Neopak 2 ml long pre-filled syringe (with a 27 gauge special-thin-walled needle) and an Ompi EZ-Fill 2 ml long pre-filled syringe (with a 27 gauge thin-walled needle), both with a rigid needle shield (RNS) and a West 2340 Flurotec Plunger Stopper.

According to at least one embodiment the dose volume may range between 1.15-2.25 ml and the drug may have a viscosity of between 1-25 cP.

### 10. Plunger (FIGS. 10 to 10M)

FIG. 10 illustrated the plunger 1000. Plunger 1000 may comprise an elongated, for example, cylindrical plunger shaft 1010. Plunger shaft 1010 may be hollow, e.g. in order to provide assembly space for drive spring 1100 and for optional spring support arm/pin 1230. On an inner surface, the plunger shaft 1010 may have at least one longitudinal rib 1060.1 to 1060.4 for guiding of the drive spring 1100. The distal part D of plunger 1000 may be closed, e.g. by a cylindrical end portion that has a smaller diameter compared to the diameter of plunger shaft 1010, e.g. in order to interface with a complementary or essentially complementary recess in the plunger stopper 910.

The proximal end of plunger 1000 may comprise several radial protrusions, e.g. at least two or at least three protrusions or two groups comprising at least two or at least three protrusions respectively:
A first plunger boss 1040.1 configured to interact with a profiled slot 1221.1 of drive spring holder 1200 (described in section 12), see e.g. FIGS. 10A to 10F and 10I,
A second plunger boss 1040.2 configured to interact with a plunger boss slot 506 of needle cover 500, see e.g. figures and FIGS. 10G and 10H, and
An angled plunger rib 1040.3.

The purpose of a distal edge (face) 1040.1d of first plunger boss 1040.1 is described in more detail in the description of FIG. 10I below. The purpose of a proximal end (face) 1040.9 of first plunger boss 1040.1 is described in more detail in the description of FIG. 12D below.

Moreover, optional plunger slots 1020, 1022, etc. may be arranged at the proximal end P of plunger 1010. Slots 1020, 1022 may be used to provide triggering of the clicker 1300 independent of the length of plunger 1000, e.g. directly or indirectly via a flexible arm that supports clicker 1300 in its biased state and that has protrusions adapted to fit into slots 1020, 1022. There may be at least one plunger slot 1020 or at least two plunger slots 1020, 1022, e.g. a pair of plunger slots adapted to be coupled to at least one protrusion or to a pair of protrusions of the flexible arm supporting clicker 1300. Optionally, at least one further slot may be on the lower side of plunger 1000, e.g. in order to provide a symmetrical design and to ease assembling of plunger 1000.

FIG. 10A illustrates the plunger release mechanism 1025 in a first state. The following elements of drive spring holder 1200 may be relevant:
A proximal region 1221,
A profiled slot 1221.1,
A first angled surface 1221.2 of profiled slot 1221.1,
A wall 1221.3 of profiled slot 1221.1, wall 1221.3 may extend essentially in an axial direction of drug delivery device 100 and may be arranged between first angled surface 1221.2 and a second angled surface 1221.4
The second angled surface 1221.4 of profiled slot 1221.1, and
A longitudinal edge 1234 of profiled slot 1221.1 (see also FIGS. 12A and 12B) may be positioned radially in a region in order to not to interact with first plunger boss 1040.1.

The plunger release mechanism 1025 may comprise the first plunger boss 1040.1 arranged on the plunger 1000 and the profiled slot 1221.1 in the proximal region 1221 of the drive spring holder 1200 (rear part of the device body 700). The profiled slot 1221.1 may comprise the first angled surface 1221.2 adapted to engage the first plunger boss 1040.1 to induce a torque in a first rotational direction R1 to the plunger 1000, a wall 1221.3 for limiting movement of the first plunger boss 1040.1 in the first rotational direction R1 when engaged to the first angled surface 1221.2. Furthermore, the profiled slot 1221.1 may comprise the second angled surface 1221.4 adapted to engage the first plunger boss 1040.1 to induce a torque in the first rotational direction R1 to the plunger 1000.

The first angled surface 1221.2 and/or the second angled surface 1221.4 may have an angle of inclination in a range from 30° to 70° relative to a perpendicular on the longitudinal axis A of the drug delivery device 100 which may also be the longitudinal axis of the plunger 1000. In other words, the first angled surface 1221.2 may have an angle of inclination in a range from 30° to 70° relative to the circumferential direction.

In a first state shown in FIG. 10A, the first plunger boss 1040.1 is engaged to the first angled surface 1221.2. Due to the drive spring 1100 acting on the plunger 1000, the first plunger boss 1040.1 is pressed against the first angled surface 1221.2 in a distal direction D such that a torque is induced to the plunger 1000 in the first rotational direction R1 so that the first plunger boss 1040.1 slides along the first angled surface 1221.2 until it abuts the wall 1221.3 so that rotation of the plunger 1000 in the first rotational direction R1 is halted (stopped). The first state may be used to assemble the drive sub-assembly.

Optionally, an indent 1221.15 may be arranged on a proximal face of profiled slot 1221.1. as described below, this indent may be used as a drop protection and/or as a guiding feature, for guiding the first plunger boss (rib) 1040.1 in an opposite rotational direction compared to rotational direction R1.

FIG. 10B shows the plunger release mechanism 1025 in a second state. Starting from the first state, the plunger 1000 has been moved a distance at least as long as the wall 1221.3 in the proximal direction P such that the wall 1221.3 no longer limits movement of the first plunger boss 1040.1 in the first rotational direction R1. The plunger 1000 has then been rotated further in the first rotational direction R1 so that the first plunger boss 1040.1 engages the second angled surface 1221.4, e.g. by using needle cover 500. Due to the drive spring 1100 acting on the plunger 1000, the first plunger boss 1040.1 is pressed against the second angled surface 1221.4 in a distal direction D such that a torque is induced to the plunger 1000 in the first rotational direction R1 so that the first plunger boss 1040.1 slides along the second angled surface 1221.4. If the plunger 1000 is not otherwise prevented from rotating further, the first plunger boss 1040.1 may slide down the second angled surface 1221.4 until disengaging it, allowing the plunger 1000 to advance in the distal direction D to displace the drug Dr or medicament M from the pre-filled syringe 900. However, this will happen only later, i.e. when the drug delivery device 100 is triggered using e.g. needle cover 500 and pressing it against the skin of a user.

In an exemplary embodiment, movement of the plunger 1000 from the first state in the proximal direction P and onto the second angled surface 1221.4 may be achieved by the needle cover 500 (proximal sleeve part 513) interacting with the plunger 1000, e.g. by engaging a plunger boss or rib on the plunger 1000. This may be done during final assembly, i.e. during assembling of the control sub-assembly and of the drive sub-assembly. Moreover, again, this may be different from triggering the device 100 for drug delivery.

Alternatively, other parts of drug delivery device 100 may be used for this purpose, e.g. an endplate of the drive spring holder 1200 comprising an appropriate protrusion whereby first angled surface 1221.2 may be shaped differently, e.g. slanted in the opposite direction compared to the direction illustrated in FIG. 10A to 10F and without usage of wall 1221.3. In this alternative embodiment, plunger rib/protrusion 1040.3 may be omitted or may not be present.

An exemplary embodiment of the plunger release mechanism 1025 is illustrated in more detail in FIGS. 10C, 10D, 10E and 10F. FIG. 10C illustrates the plunger release mechanism 1025 during final assembly of the control sub-assembly and of the drive sub-assembly. The needle cover 500 comprises the proximal sleeve part 513. The proximal sleeve part 513 may comprise:
The slot rib 507, e.g. an angled slot rib 507, comprising a longitudinal extension portion, e.g. slot rib 507, see FIG. 5, and a circumferential extension portion, e.g. the second ramp 507d, see FIG. 5:
A proximal face 513.2, e.g. on the circumferential extension portion (second ramp 507d),
A distal face 513.3, e.g. on the circumferential extension portion (second ramp 507d),
The abutment surface 507b, e.g. on the longitudinal extension portion, e.g. on slot rib 507, and
the optional first ramp 507c, see FIG. 10H.

The proximal sleeve part 513 may comprise the plunger boss slot 506, see FIGS. 10G and 10H. The plunger boss slot 506 is described in section 5 above in more detail, e.g. comprising the proximal slot 506a and the distal slot 506b.

There may be a pair of proximal sleeve parts 513, each interacting with a group of plunger protrusions 1040.2 and/or 1040.3 respectively, e.g. in order to have symmetric forces acting on protrusions 1040.2 and/or 1040.3 and on other parts in order to prevent jamming of parts and to provide smooth operation of drug delivery device 100.

The plunger release mechanism 1025 may have essentially two functions:
a) moving plunger 1000 from its first state to a second state during assembling of control sub-assembly and drive sub-assembly, i.e. needle cover 500 is static relative to device body 700 but the device body 700 comprising the needle cover 500 is moved axially relative to the drive spring holder 1200 or vice versa, see FIGS. 10C and 10D. As mentioned above, needle cover 500 or other parts may be used for this purpose, e.g. other parts of device 100.

b) release plunger 1000, if needle cover 500 is pressed against the skin of a patient, i.e. during a relative movement of needle cover 500 relative to device body 700 (front part of housing) and relative to drive spring holder 1200 (rear part of housing), see FIG. 10F.

The plunger release mechanism 1025 may comprise the plunger 1000, the proximal region 1021, and the proximal sleeve part 513 interacting with each other. The proximal sleeve part 513 and the proximal region 1221 are configured to move only axially relative to each other, e.g. in parallel with or along the longitudinal axis A relative to each other whereas the plunger 1000 can move both in parallel with the longitudinal axis A and rotate about the longitudinal axis A see rotational directions R1 and R2. The parts of the plunger release mechanism 1025 may be essentially rigid and require no deformation in order to function correctly.

The parts arranged for engaging the plunger 1000, proximal region 1221 and proximal sleeve part 513 may comprise:
The first plunger boss 1040.1 on the plunger 1000,
The second plunger boss 1040.2 on the plunger 1000,
The angled plunger rib 1040.3 on the plunger 1000,
The profiled slot 1221.1 in the proximal region 1221 adapted to interact with the first plunger boss 1040.1,
The slot rib 507 on the proximal sleeve part 513, a proximal face 513.2 of the plunger boss slot 506 adapted to interact with the angled plunger rib 1040.3, a distal face 513.3 of the plunger boss slot 506 and a abutment surface 507b of the plunger boss slot 506 adapted to interact with the second plunger boss 1040.2.

A gap 1030 is illustrated in FIG. 10C that makes it clear that there may be a rotational offset between the two parts of the figure. However, the three protrusions of plunger 1000 may have a fixed position to each other, see dashed line 1032.

The profiled slot 1221.1 may comprise a first angled surface 1221.2 adapted to engage the first plunger boss 1040.1 to induce a torque in a first rotational direction R1 to the plunger 1000, a wall 1221.3 for limiting movement of the first plunger boss 1040.1 in the first rotational direction R1 when engaged to the first angled surface 1221.2. Furthermore, the profiled slot 1221.1 may comprise a second angled surface 1221.4 adapted to engage the first plunger boss 1040.1 to induce a torque in the first rotational direction R1 to the plunger 1000.

As mentioned above, during assembly of the drive subassembly, the plunger 1000 with the drive spring 1100 is inserted into the proximal region 1221. Once the plunger 1000 reaches a proximal position, the first plunger boss 1040.1 is axially aligned with the profiled slot 1221.1. By rotating the plunger 1000 in a second rotational direction R2 by an angle, e.g. approximately 30°, the first plunger boss 1040.1 is moved into the profiled slot 1221.1. In this position the first angled surface 1221.2 moves the first plunger boss 1040.1 against the wall 1221.3 by inducing a torque to the plunger 1000 in the first rotational direction R1 due to the drive spring 1100 biasing the plunger 1000 in the distal direction D.

In order to assemble the drug delivery device 100 finally, a syringe 900 may be inserted into the control sub-assembly which may comprise the device body 700 (front part of housing).

Afterwards, the drive sub-assembly is inserted into the control sub-assembly in the distal direction D. The proximal region 1221 and the device body 700 may comprise snap connections to lock them together when assembled. During the final assembly of the drug delivery device 100 the needle cover 500 and proximal sleeve part 513 therewith may be partially depressed to allow initiation of the plunger release mechanism 1025 from the first state to the second state, e.g. by an assembly jig (not illustrated) or in a different way. Initiation is different from triggering of the plunger release mechanism 1025.

FIG. 10D illustrates the plunger release mechanism 1025 during the final assembly. Exemplary, the slot rib 507, especially proximal face 513.2, proximally abuts the angled plunger rib 1040.3 thereby inducing a torque to the plunger 1000 in the first rotational direction R1 and pushing the plunger 1000 in the proximal direction P so that the first plunger boss 1040.1 moves along the wall 1221.3 until it disengages from the wall 1221.3. This action is the priming of the device. Due to the induced torque, the first plunger boss 1040.1 moves in the first rotational direction R1 and engages the second angled surface 1221.4. The depression of needle cover 500 and of the proximal sleeve part 513 therewith may cease and, due to the first plunger boss 1040.1 engaging the second angled surface 1221.4 and the drive spring 1100 acting on the plunger 1000 in the distal direction D, the plunger 1000 may rotate further in the first rotational direction R1.

As the needle cover 500 and consequently proximal sleeve part 513 is not being depressed further, it may move in the distal direction D relative to the device body 700, e.g. under the action of a needle cover spring 600 (sleeve spring, not illustrated). This movement may be limited by the second plunger boss 1040.2 abutting the distal face 513.3 on the slot rib 507. Further rotation of the plunger 1000 in the first rotational direction R1 may be prevented by the second plunger boss 1040.2 abutting the longitudinal face of the slot rib 507. The load of the drive spring 1100 may be resolved within the proximal region 1221 by the first plunger boss 1040.1 engaging the profiled slot 1221.1. This state of the plunger release mechanism 1025 i.e. second state, is illustrated in FIG. 10E.

A sequence of operation of the drug delivery device 100 may be as follows:

The user removes the cap 200 and the cap lid 300 pulling it in the distal direction D away from the device body 700. Removal of the cap 200 and the cap lid 300 may at the same time remove a protective needle shield 914 (e.g. a rigid needle shield or a soft needle shield) from the needle 908.

The needle cover 500 may be in an extended position protruding from the device body 700 in the distal direction D. The extended position may be defined by the second plunger boss 1040.2 proximally abutting the distal face 513.3 of the slot rib 507.

The user may then press the drug delivery device 100 with the needle cover 500 ahead against an injection site, e.g. a patient's skin thereby moving the needle cover 500 from the extended position towards a retracted position against the bias of the needle cover spring 600.

FIG. 10F is a schematic view of the plunger release mechanism 1025 after depression of the needle cover 500 into the retracted position. As the needle cover 500 is being moved from the extended position towards the retracted position the second plunger boss 1040.2 moves (starting from the position shown in FIG. 10E) relative to the needle cover 500 in the distal direction D guided along the abutment surface 507b of the slot rib 507.

In an exemplary embodiment, the abutment surface 507b of the slot rib 507 may comprise an interruption or bump feature (not illustrated) for creating an increase in the force required to depress the needle cover 500 further. This may be used to indicate to the user that needle insertion would commence with further depression of the needle cover 500 and of the proximal sleeve part 513 therewith. Up until this point, the user is free to remove the drug delivery device 100 from the injection site and reposition as the needle cover 500 will re-extend to its initial position under the force of the needle cover spring 600.

If the user continues pressing the drug delivery device 100 against the injection site, the needle cover 500 is moved into the retracted position exposing the needle 908 and inserting it into the injection site.

Once the needle cover 500 is depressed into the retracted position, and the needle 908 is inserted, the second plunger boss 1040.2 has moved distally beyond the slot rib 507 such that the plunger 1000 is no longer prevented from rotating in the first rotational direction R1 due to the torque induced by the drive spring 1100 and the first plunger boss 1040.1 engaging the second angled surface 1221.4 on the profiled slot 1221.1. The plunger 1000 rotates in the first rotational direction R1 due to this torque and the first plunger boss 1040.1 comes clear of the profiled slot 1221.1 and is guided along an inner longitudinal rib 1236, see FIG. 10I. The plunger 1000 is thus released and advances the plunger stopper 910 in the distal direction D displacing the drug Dr or medicament M from the syringe 900 through the needle 908. The release of the first or second plunger boss 1040.1, 1040.2 may provide audible feedback that delivery of the medicament has started.

FIG. 10G is a schematic detail view of the plunger release mechanism 1025 after final assembly and prior to depression of the needle cover 500 and of the proximal sleeve part 513 therewith, i.e. plunger 1000 is in the second state. FIG. 10G is a view onto the inside of the proximal portion of the elongated arm of needle cover 500, especially onto the inside of proximal sleeve part 513. Movement of the needle cover 500 in the distal direction D relative to the device body 700 may be limited by the second plunger boss 1040.2 abutting the distal face 513.3 on the slot rib 507. Further rotation of the plunger 1000 in the first rotational direction R1 may be prevented by the second plunger boss 1040.2 abutting the abutment surface 507b of the slot rib 507.

FIG. 10H is a schematic detail view of the plunger release mechanism 1025 during depression of needle cover 500 and of the proximal sleeve part 513 therewith. FIG. 10H is a view onto the inside of the proximal portion of the elongated arm of needle shroud 500, especially onto the inside of proximal sleeve part 513. As the proximal sleeve part 513 is being moved from the extended position towards the retracted position in the proximal direction P the second plunger boss 1040.2 moves (starting from the position shown in FIG. 9) relative to the needle cover 500 in the distal direction D guided along the abutment surface 507b of the slot rib 507.

If the user continues pressing the drug delivery device 100 against the injection site the needle cover 500 is moved into the retracted position exposing the needle 908 and inserting it into the injection site.

Once the needle cover 500 is depressed into the retracted position and the needle 908 is inserted, the second plunger boss 1040.2 has moved distally beyond the slot rib 507 such that the plunger 1000 is no longer prevented from rotating in the first rotational direction R1 due to the torque induced by the drive spring 1100 and the first plunger boss 1040.1 engaging the second angled surface 1221.4 on the profiled slot 1221.1. The plunger 1000 rotates in the first rotational direction R1 due to this torque and the first plunger boss 1040.1 comes clear of the profiled slot 1221.1. The plunger 1000 is thus released and advances the plunger stopper 910 in the distal direction D displacing the drug/medicament Dr/M from the syringe 900 through the needle 908.

In another embodiment of the plunger release mechanism 1025, in addition to the embodiment described above, a first ramp 507c is provided on the proximal sleeve part 513. As the proximal sleeve part 513 approaches the retracted position, the first ramp 507c engages a rib or boss on the plunger 1000, e.g. the angled plunger rib 1040.3 to actively rotate the plunger 1000 in the first rotational direction R1. If the plunger 1000 should not rotate spontaneously due to the features of the previous embodiments, the additional first ramp 507c will induce rotation of the plunger 1000.

During normal use, the plunger 1000 will release as in the previous embodiments. The first ramp 507c is positioned to only interact with the angled plunger rib 1040.3 if the plunger 1000 has not spontaneously rotated near the end of the depression of the needle cover 500 and of the proximal sleeve part 513 therewith. The skilled person will readily understand that the embodiments would likewise work if only one of the rib or boss on the plunger 1000, e.g. the angled plunger rib 1040.3, or the first ramp 507c was ramped or angled. The same applies to proximal face 513.3.

Another benefit of the further embodiment, i.e. usage of first ramp 507c, is that it provides additional guidance of the plunger 1000 movement as it activates.

In another exemplary embodiment, the first ramp 507c engaging the rib or boss on the plunger 1000, e.g. the angled plunger rib 1040.3, may be the only way to rotate the plunger 1000 out of engagement with the profiled slot 1221.1. For example, the profiled slot 1221.1 may not have an angled surface causing the plunger 1000 to rotate in the first rotational direction R1 out of engagement with the profiled slot 1221.1. In an exemplary embodiment, the profiled slot 1221.1 may only have a transversal surface towards the distal direction D and transversally oriented relative to the longitudinal axis A. The transversal surface may have a detent or bump. In another exemplary embodiment the profiled slot 1221.1 may only have an angled surface causing the plunger to rotate in the second rotational direction R2 maintaining the first plunger boss 1040.1 engaged within the profiled slot 1221.1.

In an exemplary embodiment, the drug delivery device 100 may be an auto-injector.

FIG. 10I illustrates an inner longitudinal rib 1236 arranged at the inside of at least one syringe support arm 1202 of drive spring holder 1200 (see also FIGS. 12A and 12B). In other words, the inner longitudinal rib 1236 may be arranged at a radial inward facing surface of the arm(s) 1202 of drive spring holder 1200. A distal edge/face 1040.1.d of first plunger boss 1040.1 abuts a proximal face 1239 of a gliding face 1238 on the longitudinal rib 1236 in the second state of plunger 1000, e.g. rib 1236 may have a holding function for holding plunger 1000 against the biasing force of the drive spring 1100. Proximal face 1239 may be slanted such that plunger 1000 is further rotated if there is no additional support. However, second plunger boss 1040.2, 1040.2a and 1040.2b, is supported on rib 507a and therefore further rotation of plunger 1000 is prevented as long as the drug delivery device 100 is not fired. If the plunger release mechanism 1025 is triggered for injection by moving needle cover 500 proximally relative to device body 700 and relative to drive spring holder 1200, plunger 1000 is allowed to rotate, i.e. free rotation in direction R1 of second plunger boss 1040.2, see FIG. 10H, and first plunger boss 1040.1 may slide distally via gliding face 1238. Guiding ribs may be used to guide further distal movement of plunger 1000, e.g. by guiding first plunger boss(es) 1040.1.

Thus, longitudinal edge 1234 may not interfere with first plunger boss 1040.1. In other words, the longitudinal edge 1234 may be arranged at a position that is further outwards in a radial direction than the position of inner edges of first angled surface 1221.2 and second angled surface 1221.4 and first plunger boss 1040.1, such that plunger boss 1040.1 does not contact longitudinal edge 1234.

FIG. 10J illustrates a perspective view of a plunger 1000 according to a second embodiment. Plunger 1000 may be used for expelling a drug Dr, M from a drug container 900. Plunger 1000 may comprise an elongated shaft 1010, e.g. an elongated plunger rod 1010, e.g. forming a main body of the plunger, that extends from a proximal end 1011p of the plunger 1000 into the direction of a distal end 1011d of the plunger 1000. The distal end 1011d may be configured to forward a force during expelling of the drug Dr, M.

Optionally, at least one triggering feature TF may be arranged within the shaft 1010 or on the shaft 1010. The triggering feature TF may be configured to allow release of the plunger 1000 from other parts of a drug delivery device 100 in order to start expelling the drug Dr, M from the drug container 900, e.g. a pre-filled syringe 900. Two pairs 1042a, 1042b of plunger ribs may be used as triggering feature TF in the second embodiment of the plunger 1000, e.g. the same as in the first embodiment as illustrated in FIG. 10. At least one common rib CR may be used as a basis to arrange the two pairs 1042a, 1042b of plunger ribs on the plunger 1000, e.g. on the plunger shaft 1010. However, optionally, further radially extending ribs 1046 to 1049 and/or a support rib SR comprising e.g. a rounded supporting feature RF may be used to reinforce triggering feature TF as is described below in more detail.

Optionally, plunger 1000 may comprise at least one interacting feature IF that is configured to interact with a drive source that generates the force for expelling the drug Dr, M. In the second embodiment, again a drive spring 1100 may be used as drive force. Interacting feature IF may comprise an inner elongated cavity 1059 within plunger 1000, more specific within shaft 1010. Moreover, interacting feature IF may comprise inner longitudinal ribs 1060.1 to 1060.4, a slanted face 1075 as well as other optional features as is described below in more detail, see FIGS. 10L and 10M as well as corresponding descriptions.

Additionally or alternatively, plunger 1000 may comprise at least one auxiliary structure AS or at least one group 1050a, 1050b comprising at least two auxiliary structures AS. The at least one auxiliary structure AS or the group 1050a, 1050b comprising the at least two auxiliary structures AS may be configured to enable automatic recognition of at least one of a position of the plunger 1000 and/or of a movement of the plunger 1000, e.g. during testing of a device 100 comprising the plunger 1000. For example, the at least one auxiliary structure AS may be a circumferentially extending auxiliary structure extending at least partially around the circumference of the shaft 1010, for example, at least around a quarter of the circumference of the shaft 1010. However, other types of auxiliary structures may be used as well, e.g. axially extending structures. Group 1050a may comprise from distal to proximal: a groove 1051.1a, a groove 1051.2a and a groove 1051.3a. Group 1050b may comprise from distal to proximal: a groove 1051.1b, a groove 1051.2b and a groove 1051.3b.

The grooves 1051.1a, etc., may have several functions in addition to a testing function, e.g. also a visual feedback function as described later on.

A method for batch testing of a drug delivery device 100 comprising the plunger 1000 or any other plunger comprising appropriate auxiliary structures AS may comprise:
Producing a batch of the plunger 1000 and/or of the drug delivery device 100, for example, using at least one injection molding mold,
Assembling the drug delivery device 100,
Testing the drug delivery device 100, wherein testing may comprise usage of a camera (e.g. a high speed camera and/or using a shutter) in order to detect auxiliary structures AS and to recognize the position of the plunger 1000, for example, automatically, during dose (drug Dr, M) expelling and/or at the end of dose (drug Dr, M) delivery,
Performing a quality control based on at least one result of the test.

A batch may comprise several parts that are produced using the same machines/molds and/or cavity, etc. A batch may comprise a number of parts within the range of 10 to 1000, or in the range of 100 to 500.

The quality control may be a statistical quality control, e.g. defining how many devices have to be tested during production in order to secure the quality, e.g. when and how many devices have to be tested, for example, according to an approved testing plan.

For example, the auxiliary structures AS, e.g. grooves 1051a, 1051.2a, etc., are arranged at an angular position of the shaft 1010 that enables to view the auxiliary structures AS through at least one sidewall window 710 of the drug delivery device 100. However, other arrangements are possible as well, e.g. if an appropriate radiation is used to recognize the plunger position/movement, e.g. a radiation that goes through housing or body 700.

Optionally, the plunger 1000 may comprise at least one lateral opening 1052.1a, 1053 or at least two lateral openings 1052.1a, 1052.1b, for example, on opposite lateral sides of the shaft 1010. The at least one lateral opening 1052.1a, 1053 or the at least two lateral openings 1052.1a, 1052.1b may be configured to allow removal of at least one auxiliary part of a mold used to produce the plunger 1000. The at least one auxiliary molding part may be configured to hold an elongated further auxiliary molding part, e.g. comprising a rod or pin or consisting of a rod or pin, laterally during injection of plastic into the mold. The further auxiliary molding part (e.g. rod or pin) may be an elongated part that may define the inner contour or at least parts of the inner contour of the inner elongated cavity 1059 of the plunger 1000. The inner contour/cavity 1059 of the plunger 1000 may comprise an inner elongated cavity hole, especially an essentially cylindrical hole, for example, comprising a slight draft angle which may facilitate removal of the further auxiliary molding part (e.g. rod or pin) after injection of the plastic material into the mold and appropriate cooling time.

According to the second embodiment, two pairs 1052a, 1052b of molding slots may be used. Pair 1052a may comprise slots 1052.1a and 1052.2a. Pair 1052b may comprise slots 1052.1b and 1052.2b. However, it is also possible to use only one of the pairs 1052a, 1052b of molding slots or to use only one slot on each lateral side of plunger 1000, e.g. overall only two molding slots. Thus, it is possible to use only slots 1052.1a and 1052.1b or to use slots 1052.2a and 1052.2b. Slots 1052.2a and 1052.2b are described in more detail below, see FIG. 10L and corresponding description.

For example, the lateral opening(s) 1052.1a, etc., 1053 are arranged at an angular position of the shaft 1010 that prevents that the lateral opening(s) 1052.1a, etc., 1053 are visible in the at least one sidewall window 710 (drug viewing window) of the drug delivery device 100. However, other positions are possible as well, see FIG. 10J, opening 1053 at the top surface of shaft 1010, e.g. at the same angular position as the middle of upper auxiliary structure AS, 1050a, 1051.1a, etc.

A method for producing the plunger 1000 or any other plunger may comprise:
Preparing a mold for producing at least one plunger 1000 or of a plurality of plungers 1000, wherein the mold comprises two main parts that are configured to be pressed together during molding and wherein the two main parts define the outer contour of at least one of the plungers 1000. An inner contour of the inner elongated cavity 1059 of the plunger 1000 may be defined by an elongated first auxiliary molding part (e.g. comprising a rod or pin or consisting of a rod or pin), for example, arranged onto a first slider that is part of the mold. The mold may comprise at least one second auxiliary molding part that is configured to hold the elongated first auxiliary molding part (e.g. pin or rod) laterally during injection of a plastic material 1090 into the mold, for example, at a free end and/or at a middle portion of the elongated first auxiliary molding part. The at least one second auxiliary molding part may be, for example, integrally arranged onto the mold, i.e. no separate slider may be used here, although alternatively second sliders may be used here. However, each slider may make the mold and therefore the production more complex.
Closing the two main parts of the mold prior to, during or after sliding the first slider (and optionally the second sliders if any) into its molding position, whereby the at least one second auxiliary molding part holds the first auxiliary molding part (e.g. rod or pin) laterally.
Injecting plastic material 1090 into the mold, thereby forming the at least one plunger 1000, wherein the plunger 1000 comprises the at least one molding slot(s) 1052a, 1052b, 1052.1, etc. or at least one other molding opening 1053 at a position defined by the at least one second auxiliary molding part,
Optionally cooling, e.g. forced by using a liquid cooling medium within cooling cavities of the mold or free, e.g. without using a separate cooling medium different from environmental air, for example, using mainly or only heat conduction within the mold.
Opening of the two main parts of the mold and sliding back the first slider (and the second slider(s) if any) into a position that may allow ejection of the at least one plunger 1000 out of the mold,
Ejecting the at least one plunger 1000 out of the mold after opening of the two main parts.

The accuracy of the plunger 1000 may be high due to the usage of the second auxiliary molding part(s) which hold the first auxiliary molding part (e.g. rod or pin), especially at the free end thereof, e.g. in order to prevent displacement during injection of the hot plastic material 1090 under high pressure into the mold.

Optionally, the plunger 1000 or any other plunger may comprise at least one holding structure 1043, 1040.1, for example, a rib 1040.1 comprising a proximal axially extending portion 1045 and a distal supporting portion 1044 that has a greater angular width relative to the width of the axially extending portion 1045. The holding structure 1043 may be configured to interact with a further holding structure 1221.1 (profiled slot) on a part of a drug delivery device 100 such that the plunger 1000 is hold securely within the further holding structure 1221.1 (profiled slot) in a state in which it is biased by a drive spring 1100. For example, an axial length of the axially extending portion 1045 that is greater than an axial length of the distal supporting portion 1045, e.g. greater by factor 2 or greater by factor 3, for example, less than factor 10 as an example.

Optionally, plunger 1000 may comprise the triggering feature TF that is mentioned above. The triggering feature TF may consist or may comprise at least one group 1042a, 1042b, e.g. at least one pair 1042a, 1042b of at least two outwardly directed ribs 1040.2, 1040.2a, 1040.2b; 1040.3, 1040.3a, 1040.3b. Pair 1042a may comprise the ribs 1040.2a and 1040.3a. Pair 1042b may comprise ribs 1040.2b and 1040.3b.

The respective ribs 1040.2, 1040.2a, 1040.2b; 1040.3, 1040.3a, 1040.3b of the at least one group (e.g. pair) 1042a, 1042b may be positioned at the same angular positions or with less than 10 degrees angular offset relative to each other. The respective ribs 1040.2, 1040.2a, 1040.2b; 1040.3, 1040.3a, 1040.3b of the at least one group 1042a, 1042b may have different axial positions, for example, within a distance of less than 15 mm or less than 10 mm. The respective ribs 1040.2, 1040.2a, 1040.2b; 1040.3, 1040.3a, 1040.3b may also extend in the axial direction. The respective ribs 1040.2, 1040.2a, 1040.2b; 1040.3, 1040.3a, 1040.3b may have a minor angular or circumferential extension compared to their radial extension and/or to their axial extension.

The at least two outwardly directed ribs 1040.2, 1040.2a, 1040.2b; 1040.3, 1040.3a, 1040.3b of the at least one group 1042a, 1042b may be arranged on a respective common rib CR, CRa, CRb that may extend axially with regard to the longitudinal axis of the shaft 1010.

For example, at least one supporting rib SR may be arranged on the respective common rib CR, CRa, CRb. The supporting rib SR may extend axially and oblique to the at least two outwardly directed ribs 1040.2, 1040.2a, 1040.2b; 1040.3, 1040.3a, 1040.3b of the adjacent one of the at least two groups 1042a, 1042b, for example, including an angle in the range of 80 degrees to 100 degrees, e.g. of about 90 degrees or of 90 degrees with the more proximally P arranged rib(s) 1040.3a and/or 1040.3b.

For example, the at least one supporting rib SR may comprise a rounded supporting feature RF comprising a curved shape with the curve extending from an axial position equal to the axial position of a proximal end of the first rib, e.g. distal rib (e.g. 1040.2a, 1040.2b), of the at least one group 1042a, 1042b to an axial position equal to the axial position of the proximal end of the second rib, e.g. proximal rib (e.g. 1040.3a, 1040.3b) of the respective one of the at least one groups 1042a, 1042b.

The following radially extending ribs may be used, e.g. for reinforcement purposes:
A rib 1046 at the distal end of common rib CR, CRa, CRb, for example, ending on a distal part of rib 1040.2a, 1040.2b,
A rib 1047 at a middle portion of common rib CR, CRa, CRb, for example, ending on a proximal part of rib 1040.2a, 1040.2b; rounded supporting feature RF may start here,
A rib 1048 on the opposite side of common rib CR, CRa, CRb compared to the side on which the ribs 1046 and 1047 are arranged, for example, ending on a distal part of the angled rib 1040.3a, 1040.3b, and
A rib 1049 on the opposite side of common rib CR, CRa, CRb compared to the side on which the ribs 1046 and 1047 are arranged, for example, ending on a proximal part of the angled rib 1040.3a, 1040.3b; rounded supporting feature RF may end here.

Corresponding ribs may be used onto pair 1042b of plunger ribs 1040.2b and 1040.3b. In general, a rotation symmetry of the plunger 1000 can be in order to ease production (e.g. less warping during molding) and/or assembly, e.g. no further specifics for the mounting direction(s).

Optionally, plunger 1000 may comprise or may consist of a glass filled or glass-fiber filled plastic material 1090, for example, a glass-filled or glass-fiber filled polyamide, for example, glass filled polyamide PA 66. The glass-filled material may have a portion of glass or glass fibers in the range of 23 mass percent to 43 mass percent, or in the range of 30 mass percent to 36 mass percent, e.g. 33 mass percent. For example, DuPont Zytel FGFE5171, especially FGFE5171NC010C may be used which comprises 33 mass percent glass or glass fibers. Alternatively, volume percent may be used instead of mass percent in the ranges or values mentioned above. Other materials may be used as well, e.g. PA (Polyamide, Nylon) 6. PA (Polyamide, Nylon) 66 may be especially well adapted as a material for a medical device, especially for a plunger 1000 that is part of a medical device 100 as it is also appropriate for usage in food industry. Moreover, molding characteristic is excellent.

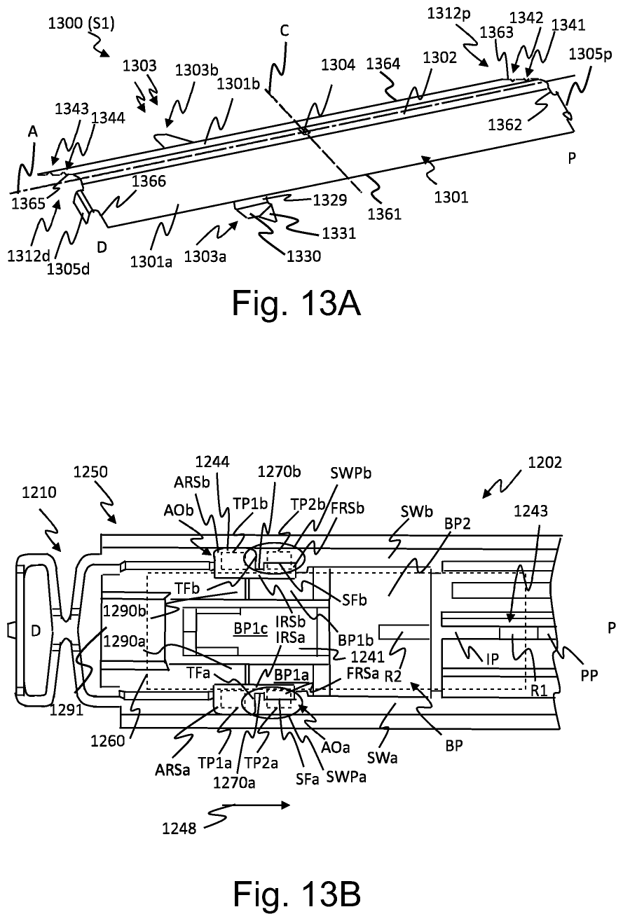

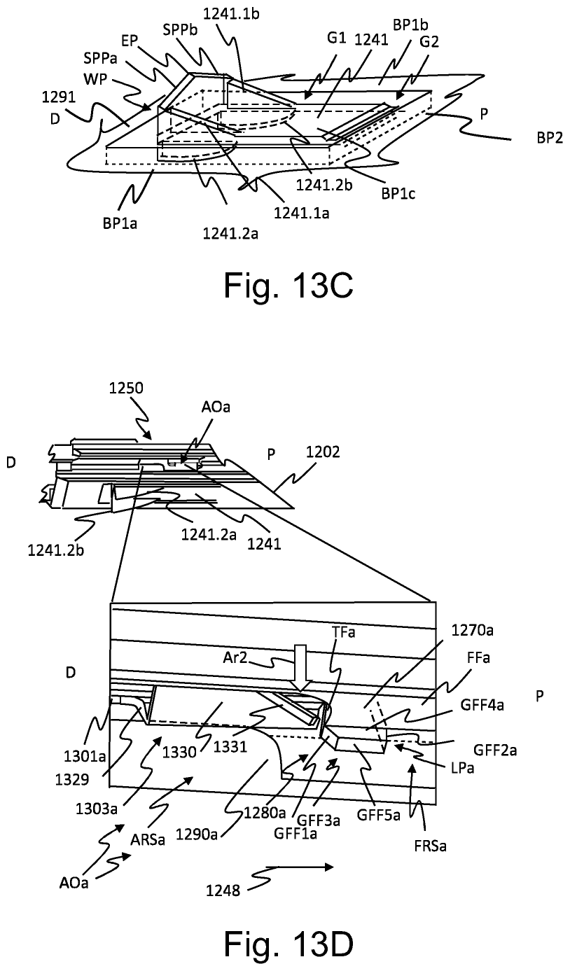

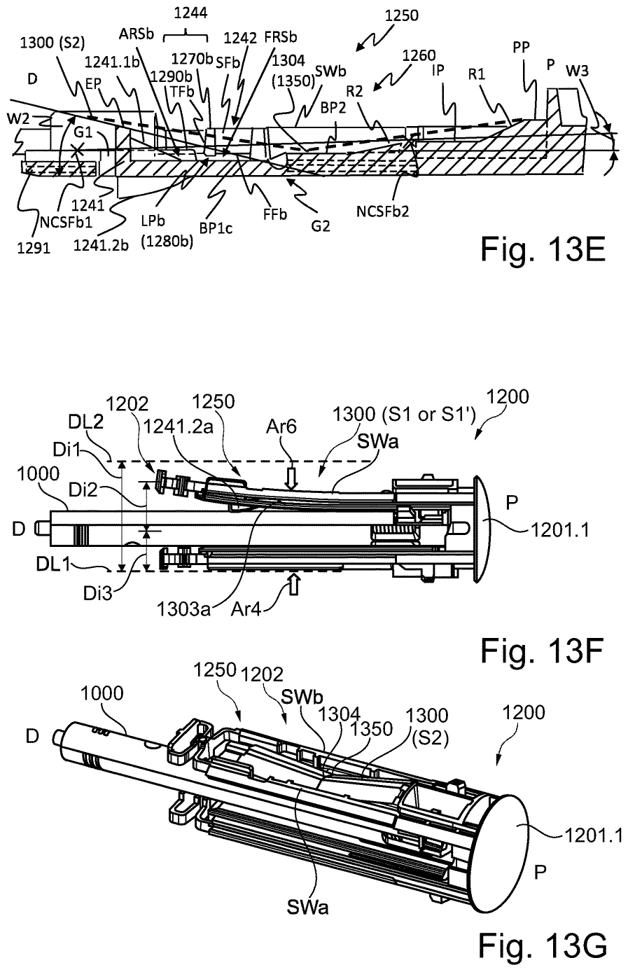

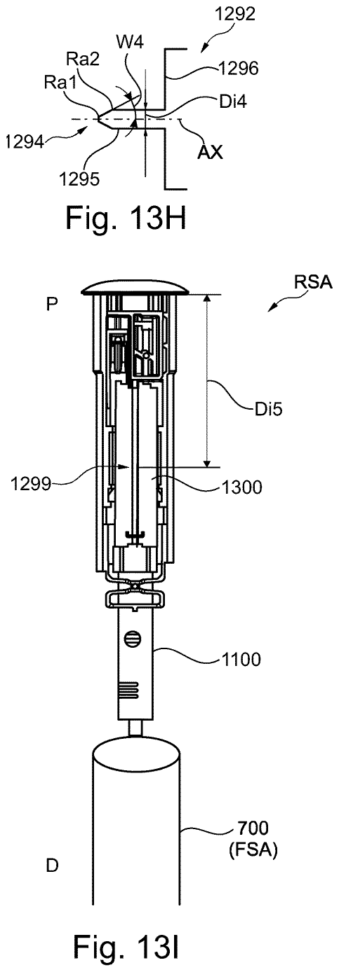

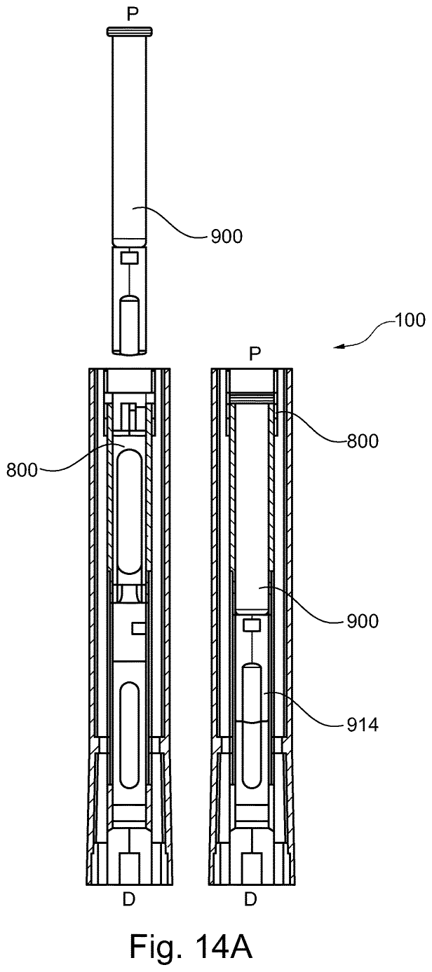

Optionally, plunger 1000 may comprise at least one axially extending cutout (e.g. a slot) 1020a, 1022a, 1020b, 1022b or at least two longitudinal cutouts (e.g. slots) 1020a, 1020b. The at least one axially extending cutout 1020a, 1020b may or the at least two longitudinal cutouts 1020a, 1022a, 1020b, 1022b may be arranged within a proximal portion of the shaft 1010. For example, the at least one axially extending cutout 1020a, 1022a, 1020b, 1022b or the at least two longitudinal cutouts 1020a, 1022a, 1020b, 1022b may be configured to interact with a support arm 1241 as explained in more detail below, e.g. see FIGS. 13B to 13E and corresponding description. Especially, the support arm, e.g. support arm 1241, may be configured to interact and/or to trigger an audible and/or indicator 1300 of a drug delivery device 100, see e.g. FIG. 13A and corresponding description.

Pairs of cutouts may be used. A first pair may comprise cutouts 1020a, 1022a. A second pair may comprise cutouts 1020b, 1022b. If only one audible and/or indicator 1300 is used only one pair may be used to trigger audible and/or indicator 1300. The other pair may not be present or may be present, e.g. in order to provide rotation symmetry of plunger 1000, especially of shaft 1010. Alternatively, only one inwardly directed rib may be used on flexible support arm 1241 or on flexible mounted support arm 1241. In this case, only one cutout may be used to interact with the single radially inwardly directed rib on support arm 1241. A further cutout may be arranged on the other side of shaft 1010. Alternatively, only one cutout is used.

Cutouts 1020a, 1020b, 1022a, 1022b may be longitudinal extending cutouts (slots). The slant of side faces of the first cutout of a pair may be different from the slant of the side faces of the second cutout of the pair, e.g. in order to prevent usage of sliders.

The features mentioned above on plunger 1000 may enable a plurality of functions. Thus, the plunger 1000 may be a multi-functional part of the drug delivery device 100, especially if all of the functions are realized. There may be synergistic technical effects of combinations of these functions, especially if further functions are considered as described below with reference to FIGS. 10 K to 10M.

FIG. 10K illustrates a distal view of the plunger 1000 according to the second embodiment. Plunger 1000 may comprise at least one identification mark 1080, for example, including at least one letter, at least one digit and/or at least one other symbol on the plunger 1000, for example, on the shaft 1010, for example, on a distally facing surface 1014 of the shaft 1010.

Plunger 1000 may comprise at least two, at least three or at least four identification marks 1080.1 to 1080.4 on the distal face 1014 of the shaft 1010, for example, on a distal face 1014 that is outwardly bordered by a distal end of the shaft 1010 and that is inwardly bordered by the proximal part of a tip portion 1012 of the plunger 1000.

In the embodiment, four marks 1080.1 to 1080.4 are arranged on annular face 1016. The four marks 1080.1 to 1080.4 may have equidistant spaces between marks that are adjacent to each other angularly. The identifier “600×” may be indicated by marks 1080.1 to 1080.4, e.g. in order to indicate a special mold and/or a special set of molds (e.g. for all parts of drug delivery device 100) and/or a special cavity within a mold. The value of the identifier “600×” may, only to give an example, indicate that the sixth cavity was used to produce the part.

At least one further marking 1082 may be arranged on other parts, e.g. on drive spring holder 1200, especially on base 1202 of drive spring holder 1200. The same value of the marking or the same marking may be used on several parts of the same drug delivery device 100, indicating that dedicated cavities and/or molds that were used to produce the parts of the drug delivery device 100, see e.g. marking “600×”. Thus, it is an option to combine e.g. only parts that are produced in the sixth cavity of the molds of a dedicated set of molds. Alternatively, to give a further example, always the plunger 1000 produced in the first cavity of a plurality of cavities for the production of plungers 1000 may be assembled with the drive spring holder 1200 produced in the second cavity of a plurality of cavities for the production of drive spring holders 1200.

Usage of markings may enable better control of the production, e.g. better statistical control, compared e.g. to random combinations of parts within one drug delivery device. Specific molds may be used for specific parts, e.g. a mold only for plungers and another mold only for other part(s). Alternatively, different types of parts may be produced within one mold, e.g. plungers 1000 and drive spring holders 1200 or other part of the drug delivery device.

A method for marking of a plunger, e.g. of plunger 1000, may comprise:
Preparing a mold for producing at least one plunger 1000 or of a plurality of plungers 1000, wherein the mold may comprise at least one cavity for the production of the at least one plunger 1000 or a respective cavity for the production of a plurality of plungers 1000,
Marking the at least one cavity by using at least one of grooves or protrusions to print at least one letter, digit or other symbol onto the at least one plunger or onto each plunger of the plurality of plungers, for example, different marks 1080.1 to 1080.4 for different cavities, for example, at least a part of the mark 1080.1 to 1080.4 indicates or is an identifier of the mold and/or cavity or wherein at least one mark comprises an identifier of the mold and/or cavity of the mold,
Using the mold for the production of the at least one plunger 1000, and
For example, tracing the mold and/or the cavity that was used to produce the plunger 1000 for at least one of the plungers 1000 produced, e.g. as part of a quality control method, for example, involving storing digital data, e.g. related to the marks or markings.

Replaceable inserts may be used to ease manufacturing of the mold and markings and/or to be able to change the markings in an easy way if necessary or to omit the markings if appropriate.

A similar method may be used for marking of parts of a device (any mechanical operating device, a housing, etc.), especially of a drug delivery device 100 comprising:
Preparing at least two molds for producing at least two different parts of a drug delivery device 100, for example, including a plunger 1000, wherein the respective mold may comprise at least one cavity for the production of the respective at least one part,
Marking the at least one cavity by using at least one of grooves and protrusions to print at least one letter, digit or other symbol onto the at least one part, for example, different marks 1080.1, 1080.4 for different cavities and/or different marks 1080.1, 1080.4 for different molds, for example, at least a part of the respective mark 1080.1, 1080.4 may indicate or may be an identifier of the mold or wherein at least one mark may comprise an identifier of the mold and/or cavity of the mold,
Using the molds for the production of at least one drug delivery device 100,
Assembling the drug delivery device 100, and
For example, tracing the mold and/or the cavity that was used to produce the device 100 for at least one of the devices 100 produced, e.g. as part of a quality control method, thereby e.g., storing digital data related to the marks or markings,

In FIG. 10K, a tool parting plane TPP is illustrated, i.e. the plane at which the two halves of the mold may contact each other physically. However, other arrangements of the TPP are possible as well. An injection point may be arranged on the distal face 1014 of the plunger tip portion 1012 or on any other appropriated location on the plunger 1000.

In an alternative embodiment, a further mark may be arranged on the distal face 1014 of plunger tip 1012, e.g. if the injection point is not arranged on this distal face 1014. According to a further embodiment at least one mark may be arranged on the distal face of the plunger tip 1012 but not on annular face 1016.

FIG. 10L illustrates a cross section along a radial direction RD through the shaft 1010 of the plunger 1000 according to the second embodiment. A circumferential direction CD is also illustrated in FIG. 10L.

Further, optionally, as mentioned above, the interacting feature IF may consist or may comprise an elongated cavity 1059 within the shaft 1010. Elongated cavity 1059 may be configured to interact with drive spring 1100, for example, with a compression spring. Moreover, elongated cavity 1059 may be configured to retain spring support arm/pin 1230, see e.g. FIG. 12A.

Plunger 1000 may comprise at least two or at least three or at least four inner ribs 1060.1 to 1060.4 extending at least along one quarter, one half, three quarters or along the complete axial length of the elongated cavity 1059. The at least two ribs 1060.1 to 1060.4 may be arranged at equidistant angular positions of adjacent ribs 1060.1 to 1060.4. Four inner ribs 1060.1 to 1060.4 are arranged on the inside of the shaft 1010 in the illustrated embodiment. The proximal end of rib 1060.1 may be arranged between cutouts 1020a and 1020b or at another appropriate position. The proximal end of rib 1060.3 may be arranged between cutouts 1022a and 1022b or at another appropriate position. All inner ribs may form a group 1060 of inner ribs.

Lateral opening 1052.2a may comprise:
A strongly slanted face 1055,
A moderately slanted face 1056 relative to the slanting angle of slanted face 1055,
A side face 1057 (not illustrated in FIG. 10L, see e.g. FIG. 10J, and
A side face 1058.

The radial direction RD may be used to define the slanting angle of slanted faces 1055 and 1056, thereby using the radial direction at the inner border or edge of the respective slanted face 1055 and 1056.

The arrangement of faces 1055 and 1056 may allow usage of mold parts for forming lateral opening 1052.2a without an additional slider as is apparent in FIG. 10L considering the tool (mold) parting plane TPP, e.g. both faces 1055 and 1056 may be produced without generating an undercut with regard to the tool closing direction and the tool opening direction that are perpendicular to the tool (mold) parting plane TPP. This is possible even if the respective mold part has a round feature that interacts with mold pin or mold rod that is used to define or form inner cavity 1059. The same may apply to side faces 1057 and 1058. However, usage of additional slider(s) is possible as well, e.g. if the tool (mold) parting plane TPP is arranged at another location.

For example, all other lateral openings, e.g. 1052.1a, 1052.1b, 1052.2b may comprise the same features compared to the features of lateral opening 1052.2a.

FIG. 10M illustrates a cross section along a longitudinal direction through the shaft 1010 of the plunger 1000.

The plunger 1000, e.g. shaft 1010, may comprise within the cross section from the proximal end 1011p to the distal end 1011d at least one or all of the following features, for example, in the sequence given, and especially on an inner side of the shaft 1010:
For example, a first rounded edge 1074,
A slanted face 1075, for example, slanted relative to slanted relative to outer surface 1071 and/or to inner main surface 1077 of the shaft 1010,
For example, a second rounded edge 1076, and/or
an inner surface 1077 of plunger 1000, more specific of shaft 1010.

Especially, slanted face 1075 may have a significant influence to the generation of noise acoustic wave components generated during release of derive spring 1100. A slant angle of slanted face 1075 relative to the longitudinal axis or relative to slanted relative to outer surface 1071 may be in the range of 30 degrees to 60 degrees, in order to generate a noise comprising a small number of noise components, thereby being agreeable for the user.

Moreover, the following features are illustrated:
An outer surface 1071 of shaft 1010,
An outer edge 1072 of shaft 1010, and
a proximal surface 1073 that is directed proximally.

Additionally, or alternatively, the radius of first rounded edge 1074 and of second rounded edge 1076 may have an influence with regard to noise generation. Thus, a smaller curvature, e.g. greater radius may be used on edge 1074 and/or edge 1076, for example, relative to radius on other edges of shaft 1010, e.g. on edge 1072 and/or on edge 1074.

A drug delivery device 100 may comprise:
A plunger 1000 according to any one of the previous mentioned embodiments, and
A drug container, especially a pre-filled syringe 900, or a retaining space that is configured to retain the drug container (e.g. syringe 900), wherein the drug container (900) may store a drug (Dr, M) or may be configured to store a drug (Dr, M).

A rear sub-assembly (RSA), see e.g. FIG. 13I, may comprise:
A drive spring holder (1200) configured to hold a drive spring (1100),
A drive spring (1100), and
A plunger (1000) according to any one of the embodiments mentioned above.

Thus, the effects mentioned above may also apply to drug delivery device 100 or to rear sub-assembly (RSA).

Neither plunger 1000 according to the first embodiment, see e.g. FIG. 10, nor plunger 1000 according to the second embodiment do comprise a thread. Therefore, production is not as complex compared to the production of plungers comprising at least one thread or also a counter thread.

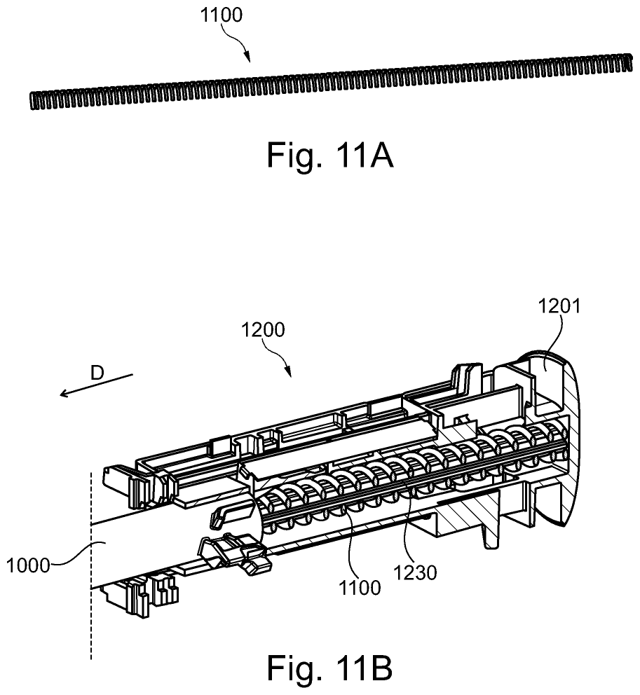

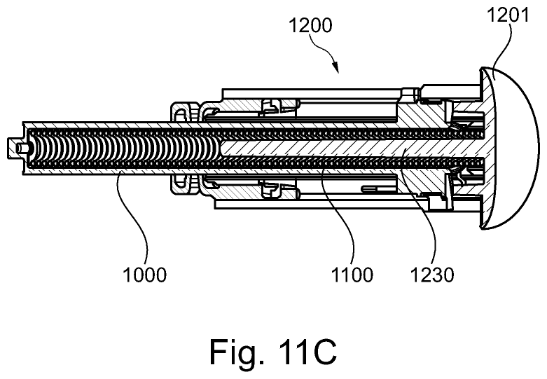

### 11. Drive Spring (FIGS. 11A to 11C)

FIG. 11A illustrates a drive spring 1100. The drive spring 1100 may be configured to provide an actuation force to the plunger 1000 in order to move the plunger 1000 in the distal direction with respect to the syringe 900 (not shown), when e.g. the first plunger boss 1040.1 of the plunger 1000 disengages from a profiled slot 1221.1 of a drive spring holder 1200, as described in sections 10 and 12.

A spring mechanism may provide the force for emptying the syringe 900. For example, the spring mechanism may comprise the drive spring 1100 which may interact with the plunger 1000 and may cause a distal movement of the plunger 1000 relative to the device body 700 and/or relative to the drive spring holder 1200 and/or relative to the syringe 900. This may result in a distal movement of the plunger stopper 910 within the syringe 900, when the plunger 1000 contacts the stopper 910. Thus, a drug can be discharged from the barrel 902 of the syringe 900. In other words, the drive spring 1100 may provide the force for the drug discharge and injection.

As illustrated in FIGS. 11B and 11C, the drive spring 1100 may surround a drive spring support arm/pin 1230 of the drive spring holder 1200 and may extend from a base 1201 of the drive spring holder 1200 in the distal direction D. A distal end of the drive spring 1100 may abut a proximal facing inner surface of the plunger 1000. In other words, the drive spring 1100 may be configured to extend inside the plunger 1000.

Drive spring support arm/pin 1230 may have an essentially circular cross section or a circular cross section. For example, at least two longitudinal guiding ribs may be arranged along the outer surface of the drive spring support arm/pin 1230. The longitudinal guiding rib may be configured to support the drive spring 1100 against a radial inward movement. There may be at least two ribs, at least three ribs or at least four ribs, for example, arranged equidistantly along the circumference of the drive spring support arm/pin 1230.

Inside the plunger 1000, the drive spring 1100 may be guided by at least one inner rib of the plunger 1000, formed at an inner surface of the plunger shaft 1010 and extending in the axial/longitudinal direction of the plunger 1000. In one embodiment, the drive spring 1100 may be guided inside the plunger 1000 by at least four of said inner ribs. The inner ribs may be arranged equidistantly around an inner circumference of the plunger shaft 1010. The inner ribs may extend along at least a part of the axial length of the plunger shaft 1010, for example, along the full axial length of the plunger shaft 1010. Alternatively, the inner ribs may have different angular offsets there between. In one embodiment, the inner ribs may extend from the slanted face 1075 of the plunger 1000 to an inner distal face of the plunger 1000. Further details are described in section 10, for example.

In one embodiment, the drive spring 1100 may be made of a high strength stainless steel. For example, the drive spring 1100 may be made of an austenitic steel with sufficient elastic properties allowing for an elastic compression of the drive spring 1100. In one embodiment, the drive spring 1100 may be made of an austenitic chromium-nickel steel. In one embodiment, the drive spring 1100 may be made of a DIN EN 1.4310 steel.

In one embodiment, the drive spring 1100 may be made of a coiled wire. A wire diameter may be selected according to the stresses that occur when the drive spring 1100 is compressed, e.g. fully compressed before the drug delivery device 100 is activated. In one embodiment, the wire may be a soap lubricated wire in order the to aid manufacturability.

In one embodiment, the drive spring 1100 may have between 10 and 150 coils or turns. In one embodiment, the drive spring 1100 may have between 20 and 120 coils or turns. In one embodiment, the drive spring 1100 may have between 40 and 100 coils or turns, e.g. 80 coils or turns.

A coil diameter may be chosen in accordance with the geometry of the plunger 1000 and the drive spring support arm/pin 1230 of the drive spring holder 1200.

In one embodiment an inner diameter of the drive spring 1100 (an inner diameter of the coils) may be between 1.5 mm (millimeter) and 6 mm, for example, between 2.0 mm and 4.0 mm, e.g. 2.5 mm.

In one embodiment an outer diameter of the drive spring 1100 (an outer diameter of the coils) may be between 2.0 mm and 8.0 mm, for example, between 3.0 mm and 6.0 mm, e.g. 4.0 mm.

A length of the drive spring 1100 and/or the wire diameter of the wire that is coiled for forming the drive spring 1100 and/or the number of coils may be chosen such that the drive spring provides a flat force profile, whilst allowing straightforward assembly. In other words, the specifics of the drive spring 1100 may be adapted to minimize the impact load at the beginning of injection and/or to minimize the forces on supporting device components during storage.

Moreover, the specifics of the drive spring 1100 may be adapted such that the drive spring 1100 provides a sufficient activation force for meeting an injection time requirement. Such a requirement may be, that the syringe 900 can be emptied in less than 30 seconds, for example, in less than 20 seconds. In one embodiment, the injection time requirement may be less than 15 seconds. Moreover, the specifics of the drive spring 1100 may be chosen according to force requirements, e.g. a maximum actuation force that can be applied to the plunger.

In one embodiment, in an unbiased state, the drive spring 1100 may have a length between 50 mm and 200 mm, for example, between 100 mm and 200 mm, e.g. 110 mm.

In one embodiment, the drive spring 1100 may be configured to provide an actuation force between 2 N (Newton) and 60 N, depending on its compression state. In one embodiment, the drive spring 1100 may be configured to provide an actuation force between 3 N and 50 N, for example, between 3 N and 40 N, depending on its compression state. In one embodiment, the drive spring 1100 may be configured to provide an actuation force between 3 N and 24 N, depending on its compression state.

### 12. Drive Spring Holder (FIGS. 12A to 12G)

FIGS. 12A and 12B illustrate a drive spring holder 1200. The drive spring holder 1200 may be configured to support the drive spring 1100 and the plunger 1000 relative to the device body 700. The drive spring holder 1200 may be configured to withstand the load of the drive spring 1100 prior to the priming of the plunger 1000, e.g. during storage of a rear sub-assembly (RSA). The drive spring holder 1200 may be further configured to compensate variations in length of the syringe 900 and to prevent a proximal movement of the syringe 900 within the drug delivery device 100. The drive spring holder 1200 may further be configured to support an audible and/or tactile indicator, e.g. a clicker 1300 (not shown) as described below.

The drive spring holder 1200 may have a base 1201 at its proximal end defining a proximal end surface 1201.1, which, in an assembled state of the drug delivery device 100, may define the proximal (rear) end surface of the drug delivery device 100.

The drive spring holder 1200 may further comprise one or more syringe support arms 1202 extending from the base 1201 in the distal direction (opposite to the proximal direction indicated by the arrow P). The syringe support arms 1202 may be arranged radially inside the device body 700, when the drive spring holder 1200 is assembled with the device body 700. The syringe support arms 1202 may be rigid, in order not to deform due to forces occurring during assembly, use or accidental drop of the drug delivery device 100.

The base 1201 may have a diameter similar to or larger than an outer diameter of the proximal end of the device body 700, such that the base 1201 of the drive spring holder 1200 cannot move distally inside the device body 700. In other words, the base 1201 of the drive spring holder 1200 may abut on the edge 732 of the proximal aperture 730 of the device body 700, when the drive spring holder 1200 is moved distally inside the device body 700.

The drive spring holder 1200 may further comprise a case lock formed by one or more deflectable snap arms 1203. The snap arms 1203 may be located at a proximal end of the drive spring holder 1200, distal to the base 1201. As illustrated in FIG. 12D, the snap arms 1203 may comprise a flexible part 1203.1 which extends in the axial direction of the drug delivery device 100, e.g. proximally. Alternatively, the flexible part 1203.1 may extend in the axial and a radial direction, such that the flexible part 1203.1 may have a radial outward inclination in the proximal direction. The case snap arms 1203 may further comprise a snap protrusion 1203.2 which protrudes from the flexible part 1203.1 in a radial outward direction. The snap arms 1203 may be pre-tensioned, i.e. biased outwardly, such that during assembly of the drive spring holder 1200 in the device body 700 (not shown), the snap arms 1203 firstly are tensed by an inward deflection caused by a contact with the inner surface of the device body 700, when the drive spring holder 1200 is moved distally inside the device body 700. Upon alignment with the proximal cut-outs 714 of the device body 700, the snap arms 1203 return to their relaxed state, thereby moving the snap protrusions 1203.2 radially outwardly to engage with or latch into the proximal cut-outs 714 in order to securely fasten the drive spring holder 1200 to the device body 700 in a first drive spring holder position (closure part position). In other words, the snap arms 1203 may be configured to form a snap-fit connection with the cut-outs 714. In the first drive spring holder position, an axial movement of the drive spring holder 1200 relative to the device body 700 may be limited/prevented due to the interaction of the snap protrusions 1203.2 and the proximal cut-outs 714. Further, in the first drive spring holder position, a rotational movement of the drive spring holder 120 relative to the device body 700 may be limited/prevented.

The drive spring holder 1200 may further comprise a drive spring support arm/pin 1230 (see FIGS. 12A-12C). A central longitudinal axis of the drive spring support pin 1230 may coincide with a central longitudinal axis of the drive spring holder 1200. The drive spring support pin 1230 may be configured to support the drive spring 1100 and the plunger 1000 in a radial direction. In other words, the drive spring support pin 1230 may center the drive spring 1100 and/or the plunger 1000 relative to the drive spring holder 1200 during assembly and before the drug delivery device 100 is activated. For example, there may be at least two, at least three or at least four longitudinal ribs arranged on an outer surface of the drive spring support pin 1230. The longitudinal ribs may be arranged equidistantly in the circumferential direction. The longitudinal ribs may support the drive spring 1100 and/or the plunger 1000 against a radial inward movement relative to the drive spring support pin 1230.

After the activation (or triggering) of the drug delivery device 100, i.e. when the plunger 1000 disengages from a profiled slot 1221.1 of the drive spring holder 1200 as explained in section 10 above, the drive spring support arm/pin 1230 may be configured to guide an axial movement of the drive spring 1100 and the plunger 1000. The arm/pin 1230 may extend from the base 1201 along at least a part of an axial length of the drive spring holder 1200, e.g. along at least 50 percent, or at least 70 percent of the axial length of the drive spring holder 1200.

In one embodiment, the drive spring support arm/pin 1230 may have a cylindrical shape along the axial direction. Alternatively, or additionally, the drive spring support arm/pin 1230 may have a conical shape along the axial direction. For example, an outer diameter of the pin 1230 may reduce in the distal direction, e.g. in order to allow deforming from a mold.

In one embodiment, the drive spring support arm/pin 1230 may be configured to guide the drive spring 1100 and/or the plunger 1000 during assembly and/or during use, i.e. during release of drive spring 1100.

In one embodiment, the profiled slot 1221.1 may be formed at a proximal end of at least one syringe support arms 1202, distal to the base 1201. The profiled slot 1221.1 may be formed next to at least one of the snap arms 1203 in a circumferential direction (see FIGS. 12A and 12B). Thus, the profiled slot 1221.1 and the snap arms 1203 may, at least partially, overlap in the axial direction. The profiled slot 1221.1 may be configured to interact with the plunger 1000, when the plunger is connected to the drive spring holder as described in this disclosure.

The drive spring holder 1200 may comprise longitudinal ribs 1236 as illustrated in FIGS. 12A and 12B, for interaction with the plunger 1000, as described in section 10 above.

As illustrated in FIGS. 12A and 12B, the drive spring holder 1200 may further comprise a clicker support structure 1240. The clicker support structure 1240 may be arranged on at least one of the syringe support arms 1202. The clicker support structure 1240 may be arranged, distally offset from the profiled slot 1221.1. The clicker support structure 1240 may define a recess into which the clicker 1300 (not shown) may be inserted, as described below, e.g. in section 13.

In one embodiment, the clicker support structure 1240 may comprise a clicker support arm 1241, at least one clicker tab restraint 1242 and a clicker rear support 1243 (see FIGS. 12A and 12B).

In one embodiment, the clicker support structure 1240 may further comprise at least one clicker mounting groove 1244.

The clicker support structure 1240 is configured to support the clicker 1300 and may support the clicker 1300 in a radial outward direction, when the plunger 1000 overlaps with the clicker support arm 1241 and the drive spring support arm 1230 in the axial direction.

The clicker support arm 1241 may be flexible and/or resilient (elastic) and/or deflectable and/or movable, for example, in a substantially radial direction of the drive spring holder 1200. The clicker support arm 1241 may be arranged at a distal end of the clicker support structure 1240, distal to the clicker rear support 1243 and the clicker tabs restraint 1242.

The clicker support arm 1241 may comprise, at least one outwardly directed ramp-like protrusion 1241.1. The outwardly directed ramp-like protrusion 1241.1 may extend radially outwardly from a radial outwardly facing surface of the syringe support arm 1202. The ramp-like protrusion 1241.1 may have a radial outward inclination in the distal direction D. In one embodiment, the outwardly directed ramp-like protrusion 1241.1 may comprise two outwardly directed ramps/ribs with a recess there between.

The clicker support arm 1241 may further comprise at least one inwardly directed ramp-like protrusion 1241.2 (see FIGS. 12A and 12B). The inwardly directed ramp-like protrusion 1241.2 may extend radially inwardly from an inwardly facing surface of the syringe support arm 1202. The ramp-like protrusion 1241.2 may have a radial inward inclination in the distal direction D. In one embodiment, the inwardly directed ramp-like protrusion 1241.2 may comprise two inwardly directed ramps/ribs with a recess there between.

The clicker support arm 1241 may be radially supported, e.g. held in radial position, by an outer surface of the plunger 1000, as long as there is an axial overlap between these components. For example, the inwardly directed ramp-like protrusion 1241.2 may abut on the outer surface of the plunger 1000, such that a radial inward movement of the flexible support arm 1241 is limited, for example, prevented.

After a proximal end of the plunger 1000 has or cutouts 1020, 1022 have passed the inwardly directed ramp-like protrusion 1241.2, e.g. when the plunger 1000 moves distally after disengaging from the abutment surface 507b, the clicker support arm 1241 may deflect and/or move radially inwardly, thereby no longer supporting the clicker 1300 in a radial outward direction. Thus, the clicker 1300 may return to its relaxed state (S1), as outlined in section 13 below, thereby generating an audible and/or tactile signal indicating end of drug Dr, M delivery.

In one embodiment, the inwardly directed ramp-like protrusion 1241.2 may be complementary to cut-out(s)/slot(s) 1020, 1022 of the plunger 1000. In this embodiment, the clicker support arm 1241 may deflect and/or move radially inwardly, when the cut-outs 1020, 1022 align with the ramp-like protrusion 1241.2. This may require less distal movement of the plunger 1000 relative to the drive spring holder 1200. Moreover, plungers of different lengths 1000 may be used in different drug delivery devices 100 without modifying the trigger mechanism for clicker 1300. The length of the plunger may determine the amount of drug Dr, M expelled during drug injection.

In one embodiment, the clicker rear support 1243 may be arranged proximally offset from the clicker support arm 1241 and the at least one clicker tab restraint 1242. The clicker rear support 1243 may comprise one or more ramp-like protrusions extending radially outwardly from the outer surface of drive spring holder 1200, for example, from a bottom surface on support arm 1202, e.g. upper support arm 1202 carrying audible indicator 1300 (clicker), as illustrated in FIG. 12A. The one or more ramp-like protrusions may have a radial outward inclination in the proximal direction P. The clicker rear support 1243 may be configured to support a proximal section of the clicker 1300 radially outwardly in a biased state of audible indicator 1300 and/or during assembling/priming of audible indicator 1300, which is described in further detail in section 13 below.

The clicker support structure 1240 as illustrated in FIGS. 12A and 12B may comprise two clicker tab restraints 1242. The clicker tab restraints 1242 may be configured to engage supporting tabs 1303a, 1303b (see e.g. FIG. 13A) of a resilient force member 1301 (see e.g. FIG. 13A) of the clicker 1300 (see e.g. FIG. 13A), as described below. In one embodiment, the clicker tab restraints 1242 may be formed as notches or grooves with a restraining portion. The restraining portion may be configured to avoid a radial outward movement of the supporting tabs 1303a, 1303b of the clicker 1300 caused by a biasing force of the clicker 1300, when the clicker 1300 is mounted to the clicker support structure 1240, e.g. in the relaxed state S1 and/or in the biased state (S2).

In one embodiment, the clicker 1300 may be mounted to the clicker support structure 1240 by inserting the supporting tabs 1303a, 1303b through the clicker mounting grooves 1244. For example, the supporting tabs 1303a, 1303b may be inserted through the clicker mounting grooves 1244 in a radial inward direction against a biasing force of the clicker 1300 until the supporting tabs 1303a, 1303b are further radially inwards than the restraining portion of the clicker tab restraints 1242. Afterwards, the clicker 1300 may be moved in the proximal direction P with respect to the clicker support structure 1240 until the supporting tabs 1303a, 1303b engage with the restraining portion of the clicker tab restraints 1242. Further details are explained in section 13 below.

In one embodiment, at least one, for example, all, of the syringe support arms 1202 comprise at least one, for example, two, guide ribs 1202.1 extending along at least a portion of the syringe support arm 1202 in an axial direction (see FIGS. 12A and 12C). The illustrated guide ribs 1202.1 may be configured to rotationally align the drive spring holder 1200 and to guide an axial translation of the drive spring holder 1200 with respect to the device body 700 during assembly of the drive spring holder 1200. After assembly, the guide ribs 1202.1 may hold the drive spring holder 1200 in its rotational position relative to the device body 700. For example, the guide ribs 1202.1 may be configured to engage the holder ribs 726 of the device body 700 in order to avoid a relative rotation between the drive spring holder 1200 and the device body 700.

In one embodiment, the drive spring holder 1200 may be inserted into the device body 700 through the aperture 730 in the distal direction D, until it forms a snap fit with the device body. In detail, due to the distal movement of the drive spring holder 1200 relative to the device body 700, an inclined surface of the snap protrusion 1203.2 comes in contact with the edge 732 of the aperture 730, causing the snap arms 1203 to radially inwardly deflect. This enables a distal movement of the drive spring holder 1200 inside the device body 700. Upon alignment of the snap protrusions 1203.2 with the cut-outs 714, the snap arms return to their non-deflected state, causing the snap protrusions 1203.2 to snap into the cut-outs 714 of the device body 700. The resulting snap-fit may limit/prevent an axial movement of the drive spring holder 1200 relative to the device body 700. During the distal movement of the drive spring holder 1200 relative to the device body 700, the base 1201 comes into contact with the edge 732 of the device body 700 and abuts thereon. Thus, a maximum distal position of the drive spring holder 1200 relative to the device body 700 may be defined.

As illustrated in FIGS. 12A to 12C, the syringe support arms 1202 may comprise flexible portions 1210 at their distal ends. The flexible portions 1210 may be formed integrally with the syringe support arms 1202.

Alternatively, the flexible portions 1210 may be connected to the syringe support arms 1202 by suitable connecting elements, e.g. by latching elements.

The flexible portions 1210 may be configured to compensate length deviations, e.g. due to manufacturing tolerances, when the pre-filled syringes 900 of different lengths are assembled into the drug delivery device 100. The flexible portions 1210 may comprise a distal end surface 1211 and a flexible body 1212.

The flexible body 1212 may be configured to deform in order to adapt the length of the syringe supports 1210 in the axial direction, when a force in the axial direction is applied to the distal end surface 1211. In other words, the flexible portions 1210 may be designed as elastic spring portions.

In one embodiment, there may be a gap between the flexible portions 1210 and the clicker support structure 1240, in the axial direction.

A syringe backstop mechanism may prevent a movement of the pre-filled syringe 900 in the proximal direction P with respect to the drug delivery device 100, when the syringe 900 is assembled into the drug delivery device 100. As illustrated in FIGS. 12A and 12C, the distal end surfaces 1211 of the flexible portions 1210 may have at least one supporting rib 1211.1 which abuts on the syringe flange 912, when the syringe 900 is assembled into the drug delivery device 100 or the syringe holder 800. In the embodiment, only or exactly one supporting rib 1211.1 may be arranged on each end surface 1211, for example, in the middle of the end surface 1211, and has a main extension direction in the radial direction. Thus, supporting rib 1211.1 may be arranged centered with respect to a longitudinal axis of the support arm 1202. As further illustrated in FIG. 12C, the contact between the supporting rib 1211.1 and the flange 912 may be a line contact. In one embodiment not illustrated, the supporting rib 1211.1 may be in surface contact or in point contact with the syringe flange 912. As illustrated in FIG. 12C, if a syringe holder 800 is used, the syringe flange 912 may be proximal to the holder flange portion 805 such that the supporting rib 1211.1 does not contact the holder flange portion 805.

In other words, the flexible portions 1210 may be configured to apply a force to the syringe 900, e.g., to its syringe flange 912, in the distal direction D in order to axially bias the syringe 900 and hold it in place. Consequently, the syringe is less prone to damage and/or compromise of its sterility barrier during transport and in the event of the device being dropped.

In one embodiment, the flexible portions 1210 may have a progressive spring characteristic curve in respect of an axial deflection. Thus, a spring force provided by the flexible portions 1210 may increase with an increasing length of the syringe 900 assembled in the drug delivery device 100. However, the flexible portions 1210 are configured to contact the syringe 900, when the syringe 900 is assembled to the drug delivery device 100 such that they may compensate variations in axial lengths of the syringes 900 used due to their axial deflection. Further, an axial movement of the syringe 900 may be prevented during storage, transportation, drop and use of the drug delivery device 100.

In one embodiment, the flexible portions 1210, and/or at least the flexible body 1212, may be formed of a resilient material, e.g. plastic.

In one embodiment as illustrated in FIGS. 12A and 12B, the flexible body 1212 of the flexible portions 1210 may have a labyrinth-shaped design with at least two axially arranged chambers 1212.1 connected by at least one connection structure 1212.2. The connection structure 1212.2 may be a web. The chambers 1212.1 may form a double-spring.

FIGS. 12E to 12G illustrate different embodiments of the flexible portions 1210 according to the disclosure.

In one embodiment, the flexible portions 1210 may be designed in the form of a multiply folded or bent arc or half-arc or spring arm. Furthermore, the flexible portions 1210 may be of an accordion-shaped, labyrinth-shaped, U-shaped, V-shaped, W-shaped or S-shaped design. The flexible portions 1210 may form a double-spring.

For example, FIG. 12E illustrates two supporting ribs 1211.1 arranged on end surface 1211. FIG. 12F illustrates two supporting ribs 1211.1 in combination with two connection structures (e.g. webs) 1212.2. Alternatively, one supporting rib 1211.1 may be combined with two connection structures (e.g. webs) 1212.2, etc. FIG. 12G shows a further embodiment of the flexible portion 1210 of a meander-shaped form with a bent flexural beam 1212.3 running in a meander-shaped manner and with connecting webs 1212.2 and supporting ribs 1211.1.

FIG. 12D illustrates a proximal part of the drive spring holder 1200. The profiled slot 1221.1 may be formed close to the base 1201 of the drive spring holder 1200 from where the syringe support arms 1202 extend. In other words, the profiled slot 1221.1 may be formed at a proximal portion of syringe support arms 1202, e.g. in the first 30 percent of their length when measured from the proximal end.

In one embodiment, only one of the syringe support arms 1202 may comprise the profiled slot 1221.1. Alternatively, more than one, or all of the syringe support arms 1202 may comprise the profiled slot 1221.1

The profiled slot 1221.1 may comprise a first angled surface 1221.2 adapted to interact with the first plunger boss 1040.1 to induce a torque in the first rotational direction R1 to the plunger 1000, when an axial force is applied to the plunger 1000 in the distal direction D. The profiled slot 1221.1 comprises a wall 1221.3 for limiting the movement of the first plunger boss 1040.1 caused by the torque in the first rotational direction R1, when the plunger boss 1040.1 is engaged to the first angled surface 1221.2 and a force in the distal direction D is applied to the plunger 1000.

In one embodiment, the profiled slot 1221.1 may comprise a second angled surface 1221.4 adapted to engage the first plunger boss 1040.1 to induce a torque in the first rotational direction R1 to the plunger 1000, when an axial force is applied to the plunger 1000 in the distal direction D and when the first plunger boss 1040.1 has rotated beyond the wall 1221.3 in the first rotational direction R1.

In other words, the first angled surface 1221.2 and the second angled surface 1221.4 together with the first plunger boss 1040.1 may be configured to convert an axial force applied to the plunger 1000 into a rotational and axial movement of the plunger 1000, e.g. a helical movement of the plunger 1000.

In one embodiment, a proximal wall 1221.14 of the profiled slot 1221.1 comprises an indent 1221.15 adapted to receive a corresponding proximal end 1040.9 (see FIG. 10) of the first plunger boss 1040.1 (see FIG. 10). The indent 1221.15 and the proximal end 1040.9 may have corresponding geometries, e.g. an L-shape with an angle greater than 90 degrees, such that a rotation of the plunger 1000 in the first rotational direction R1 relative to the drive spring holder 1200 is prevented, when the proximal end 1040.9 is received within the indent 1221.15. A ramp 1221.16 on the indent 1221.15 may be adapted to guide the proximal end 1040.9 into the indent 1221.15 and rotate the plunger 1000 in the second rotational direction R2 so that the first plunger boss 1040.1 remains angularly aligned with the first angled surface 1221.2, e.g. if the rear sub-assembly RSA is unintentionally dropped down. Alternatively, or additionally, ramp 1221.16 may ease mounting of plunger 1000 and especially of first plunger boss 1040.1 into the position illustrated in FIG. 10A. Thus, ramp 1221.16 and/or intent 1221.15 may prevent unintended distal movement of the plunger 1000 relative to the drive spring holder 1200, when the plunger 1000 is mounted in the drive spring holder 1200.

In the assembled state of the rear sub-assembly, first plunger boss 1040.1, 1040.1a, 1040.1b is arranged within the “upper half” of profiled slot 1221.1, e.g. with its distal surface 1040.1d abutting on first angled surface 1221.2. An axial clearance between the proximal end 1040.9 and the proximal wall 1221.14 may be such that the first plunger boss 1040.1 can disengage the wall 1221.3 and engage the second angled surface 1221.4 during priming of the device which is described above and below in more detail. Further details regarding the interaction between the drive spring holder 1200 and the plunger 1000 are described in section 10, for example.

In one embodiment, the drive spring holder 1200 may have a length between 30 mm (millimeter) and 100 mm in the axial direction from the proximal end surface 1201 to the distal end surface 1211. In one embodiment, this length may be between 45 mm and 70 mm, e.g. 55 mm.

In one embodiment, the proximal end surface 1201 may have a diameter between 7 mm and 40 mm. In one embodiment, the proximal end surface 1201 may have a diameter between 10 mm and 30 mm. In one embodiment, the proximal end surface 1201 may have a diameter between 15 mm and 20 mm, e.g. 16 mm.

In one embodiment, a distance between outer surfaces of the syringe support arms 1202 (the outer diameter of the drive spring holder) may be between 5 mm and 40 mm. For example, distance of outer surfaces of the syringe support arms 1202, may be between 10 mm and 20 mm, e.g. 13 mm.

In one embodiment, a distance between the radial outer surfaces of the flexible portions 1210 may be similar to a distance between the radial outer surfaces of the syringe support arms 1202. Alternatively, distance between the radial outer surfaces of the flexible portions 1210 may be smaller or bigger than the distance between the outer surfaces of the syringe support arms 1202.

In one embodiment, a distance between the radial outer surfaces of the flexible portions 1210 may be between 5 mm and 40 mm. For example, said distance may be between 10 mm and 20 mm, e.g. 13 mm.

In one embodiment, the flexible portions 1210 may have a length between 2 mm and 20 mm in the axial direction. In one embodiment, the flexible portions 1210 may have a length between 5 mm and 10 mm in the axial direction, e.g. 8 mm.

In one embodiment, the chambers 1212.1 may have a length between 1 mm and 15 mm in the axial direction. In one embodiment, the chambers 1212.1 may have a length between 1 mm and 8 mm in the axial direction, e.g. 2 mm.

In one embodiment, the chambers 1212.1 may have a width between 1 mm and 20 mm in a direction perpendicular to the axial direction. In one embodiment, the chambers 1212.1 may have a width between 5 mm and 15 mm in the direction perpendicular to the axial direction, e.g. mm.

In one embodiment, the material of the drive spring holder 1200 may be the same material as the material of the device body 700. In one embodiment, the material of the drive spring holder 1200 may be plastic. In one embodiment, the material of the drive spring holder 1200 may be a PC/ABS blend, e.g. Bayblend M850XF.

### 13. Clicker (FIG. 13A to 131)

FIG. 13A illustrates an optional audible indicator (clicker) 1300. The clicker 1300 may provide the end of dose audible feedback to the user. Audible indicator 1300 may be or may comprise a sheet metal component. The precise form of the component may be important for ensuring it primes and activates reliably whilst producing a loud noise and minimizing the drag on the plunger 1000 when held in its deflected state during injection.

EN (European Norm) 1.4310 stainless steel may be chosen for its medical compatibility and high yield strength. A resilient force member 1301 may aid priming the component in a consistent location.

Lateral tabs 1303a, 1303b may hold the clicker 1300 in place in the drive spring holder 1200. Lateral tabs 1303a, 1303b may be cranked at the interface to the long side edges 1361 and 1364 of wings 1301a, 1301b. The wing-shaped section 1301a, 1301b may aid assembly, especially the portions that are bent downwards.

FIG. 13A is a schematic view of the clicker 1300. As mentioned before, the clicker 1300 may be an audible and/or tactile indicator. The clicker 1300 may comprise a resilient force member 1301 having a substantially rectangular shape and comprising a longitudinal axis A running in parallel to the longest side of the outer circumference of the resilient force member 1301. In other embodiments, the resilient force member 1301 may have a triangular shape or any other geometrical shape suitable to generate a loud noise and/or to couple the audible and/or tactile indicator 1300 to the drug delivery device 100.

The resilient force member 1301 may be designed as a monostable leaf spring comprising a resilient material, e. g. spring steel or spring plastic. Thus, the resilient force member 1301 may be capable of residing in two states S1 and S2. That is, the resilient force member 1301 may have two different conformations or conformation states S1, S2, one of them stable with limited or no application of an external force and the other one unstable. For example, these two states S1, S2 can include a first or relaxed state S1 (or pre-assembly state, or triggered state), in which the resilient force member 1301 has a first conformation. In a second or biased state S2, the resilient force member 1301 can have a second conformation. In the present FIG. 13A, the resilient force member 1301 is in the relaxed state S1 which can correspond to the pre-assembly state as well as to a state at the end of drug delivery, after the clicker 1300 has been triggered.

The resilient force member 1301 may be plastically bent by a certain angle about the longitudinal axis A forming a longitudinal round fold 1302 with two adjacent angled wing-shaped sections 1301a and 1301b angled to each other with an angle of less than 180 degrees. The longitudinal round fold 1302 may have a bend radius in the ranges of 1.5 mm (millimeter) to 2 mm, e.g., 1.6 mm+/−0.1 mm. In other embodiments, the bend radius may be outside these ranges. This bend radius may reduce a stress impact during priming and the risk of permanent deformation. However, alternatively, a longitudinal edge may be used instead of round fold 1302.

The angle between the two adjacent angled wing-shaped sections 1301a and 1301b can be:
Between 130 degrees and 140 degrees, or
Between 140 degrees and 155 degrees, or
Between 132 degrees and 142 degrees, or
Between 134 degrees and 140 degrees, or
Between 136 degrees and 138 degrees.

In an exemplary embodiment, the angle is approximately or exactly 136 degrees or 137 degrees or 138 degrees or 148 degrees or 152 degrees.

In in the present FIG. 13A, the wing-shaped sections 1301a and 1301b are angled downwards. The longitudinal round fold 1302 is located in the center of the resilient force member 1301 running in parallel to the longitudinal axis A.

Furthermore, the resilient force member 1301 may comprise one or more supporting tabs 1303a and 1303b, especially lateral supporting tabs, projecting outwardly from a long side of at least one of the wing-shaped sections 1301a and 1301b. For example, the resilient force member 1301 may include a pair of supporting tabs 1303, wherein each wing-shaped section 1301a and 1301b may comprise one supporting tab 1303a and/or 1303b. The (lateral) supporting tabs 1303a/1303b may be respectively arranged between a notch 1304 and a distal end face 1312d of the resilient force member 1301 with respect to the longitudinal axis A in order to increase a reliability of function of the audible and/or tactile indication as well as stability under drop. Distal end face 1312d is arranged at an opposite side of clicker 1300 compared to a proximal end face 1312p. Furthermore, the lateral supporting tabs 1303a/1303b may be arranged opposite to each other with respect to a cross axis C running perpendicular to the longitudinal axis A, e.g. there may be four lateral tabs 1303.

In order to facilitate assembly of the audible and/or tactile indicator 1300 into the drug delivery device 100, the supporting tabs 1303a/1303b respectively may have a free end which is upwardly bent. The supporting tabs 1303a/1303b may have a rectangular shape, a quadratic shape or another appropriate shape. Respectively, one edge, especially one corner, of the free end 1331 of the supporting tabs 1303a/1303b may be bent downwardly and thus perpendicular to the longitudinal axis A and to the cross axis C forming a triangular tab portion thereby, see e.g. triangular portion 1331. The other corner of the free end may not be bent relative to a remaining main portion 1330 of supporting tab 1303a/1303b. Thus, the bending line of triangular portion 1331 may run diagonally through tab 1303a/1303b. Alternatively, bending about a line parallel to the diagonal may be used or bending according to another appropriate way.

A connecting portion 1329 of lateral tabs 1303a, 1303b may be double cranked, i.e. downwardly on edge 1361, 1364 and again upwardly between connecting portion 1329 and main portion 1330 of lateral tabs 1303a and 1303b. Triangular portion 1331 may have one side located at a diagonal of the rectangle (or quadrate) forming main portion 1330 and triangular portion 1331. Alternatively, triangular portion 1331 may have one side located parallel to a diagonal of the rectangle (or quadrate) forming main portion 1330 and triangular portion 1331, as illustrated in FIG. 13A. Other bending patterns are possible as well, in order to form triangular portion 1331, e.g. bending about an axis that is non-parallel to the diagonal mentioned above.

According to another embodiment, the supporting tabs 1303 may have a rectangular shape or another appropriate shape. Respectively, the free end of the supporting tabs 1303 may entirely be bent upwards in an angle about an axis running perpendicular to the longitudinal axis A and to the cross axis C. The bending line may be parallel to longitudinal axis L. The bending line may be arranged at the end of the lateral tabs 1303 that is connected with wing-shaped section 1301a or 1301b. Alternatively, the bending line may be arranged parallel to longitudinal axis A but in the middle of tab 1303a/1303b or at another appropriate location.

The resilient force member 1301 may further comprise the notch 1304 that is formed into the longitudinal round fold 1302 and that may extend transversely with respect to the longitudinal round fold 1302, e.g. perpendicular. Alternatively, a hole may be used instead of the notch 1304, e.g. a blind hole or a through hole, e.g. cylindrical hole.

The notch 1304 may be centrically arranged in the longitudinal round fold 1302 with respect to the longitudinal axis A, L. Thus, notch 1304 may be arranged at the middle between the short sides of resilient force member 1301. However, asymmetric placement of notch 1304 is also possible, e.g. nearer to the proximal side P compared to the distal side D or vice versa.

The notch 1304 may support priming of the resilient force member 1301. The notch 1304 may be configured as an opening or alternatively as a blind hole. The notch 1304 may ease elastically bending of the distal part of clicker 1300 upwards around axis C.

Further, angled tabs 1305d and 1305p may be used to prevent nesting of the clicker 1300 during part feeding, i.e. during assembly, especially during automatic part feeding. For example, nesting of several clickers 1300 may be prevented. Tabs 1305d and 1305p may have a rectangular shape, a quadratic shape or other appropriate shape. Both tabs 1305d and 1305p may be arranged at the respective short sides 1362, 1366 or 1363, 1365 of the same wing-shaped section 1301a or 1301b. Alternatively, tabs 1305d and 1305p may be arranged on different wing-shaped sections 1301a or 1301b of the same resilient force member 1301, for example, again, at opposite ends (distal, proximal). Distal end tab 1305d may be arranged on distal end face 1312d. Proximal end tab 1305p may be arranged on proximal end face 1312p. Angled tabs 1305a and 1305b may be angled by an angle in the range of 70 degrees to 110 degrees, for example, by 90 degrees. Angled tabs 1305a and 1305b may also fulfill other functions.

Audible indicator 1300 may comprise at least one indentation 1341 to 1344 (notch) on at least one of the at least two wing shape sections 1301a, 1301b. At least one indentation 1341 to 1344 may be arranged on an edge of the wing shaped sections 1301a, 1301b that is different from the edge 1361, 1364 comprising at least one of the at least two lateral tabs 1303a, 1303b. The at least one indentation 1341 to 1344 may be positioned on a position that allows to hold webs between the audible indicator 1300 and a frame of a sheet after punching out the main shape of the audible indicator 1300.

In the example, there are two indentations 1341, 1342 on edge 1363 of wing shaped section 1301b and two indentations 1343, 1344 on edge 1365 of wing shaped section 1301b. Indentations 1341 to 1344 may ease loosening of clicker 1300 from webs that hold clicker 1300 within the sheet material, e.g. metal sheet, after cutting out the outer contour of clicker 1300.

FIG. 13B illustrates an indicator holder 1250, exemplarily comprised on a drive spring holder 1200, especially on one of the syringe support arms 1202 of drive spring holder 1200. As mentioned above, drive spring holder 1200 may comprise a drive spring support arm/pin 1230 that is not illustrated in FIG. 13B as it is hidden by support arm 1202. Alternatively, indicator holder 1250 may be arranged directly on body 700. Other arrangements of indicator holder 1250 are also possible.

Indicator holder 1250 may comprise a retaining cavity 1260 that may comprise or may be configured to comprise an audible indicator 1300. The audible indicator 1300 may comprise at least two lateral tabs 1303a, 1303b that are configured to position the audible indicator 1300 within the indicator holder 1250. The retaining cavity 1260 may be defined or bordered by a bottom portion BP (described below in more detail) and configured to support the audible indicator 1300. Moreover, the retaining cavity 1260 may be bordered by two lateral side walls SWa, SWb that may be parallel to each other. There may be no distal border of the retaining cavity 1260. However, proximal part of flexible portion 1210 may be regarded as a distal border of retaining cavity 1260. A proximal border may be formed by a transversal rib (not illustrated in FIG. 13B, see e.g. FIG. 13E or 13G).

Within lateral side walls SWa, SWb, there may be arranged at least two lateral side wall portions SWPa, SWPa configured to enable or ease insertion of the audible indicator 1300 into the retaining cavity 1260 as described in detail below. The respective side wall portion SWPa, SWPb may comprise:
A first face FFa (see FIG. 13D), FFb (see FIG. 13E) directed to a final retaining space FRSa, FRSb for retaining the lateral tabs 1303a, 1303b in an assembled state of the audible indicator 1300, and/or
A second face SFa, SFb directed to the retaining cavity 1260, and/or
A third face TFa, TFb directed to an auxiliary retaining space ARSa, ARSb that is configured to retain the respective lateral tab 1303a, 1303b of the audible indicator 1300 before it is moved into the final retaining space FRSa, FRSb.

An intermediate retaining space IRSa, IRSb (e.g. a slit, especially a straight slit) may be arranged between auxiliary retaining space ARSa, ARSb and final retaining space FRSa, FRSb in order to enable an axial shift of the respective lateral tab 1303a, 1303b during assembling of the audible indicator 1300 within retaining cavity 1260.

Thus, an assembling opening AOa may comprise auxiliary retaining space ARSa, intermediate retaining space IRSa and final retaining space FRSa. An assembling opening AOb may comprise auxiliary retaining space ARSb, intermediate retaining space IRSb and final retaining space FRSb. Both assembling openings AOa and AOb may have a similar shape, especially a mirror symmetric shape that has symmetry with regard to a middle axis of indicator holder 1250 extending parallel to the side walls SWa and SWb in the middle between these two side walls SWa and SWb.

Both assembling openings AOa and AOb may allow dedicated insertion of lateral tabs 1303a and 1303b of audible indicator 1300 during assembling of the audible indicator 1300 as well as dedicated priming of audible indicator 1300 from comparably flat single V-shape to much higher double V-shape as will be explained below in detail.

Bottom portion BP of retaining cavity 1260 may comprise:
A first bottom portion (plane) which may comprise three sub-portions BP1a, BP1b, BP1c arranged parallel to the first face FFa (see FIG. 13D), FFb (see FIG. 13E), and
A second bottom portion (plane) BP2 arranged parallel to the first face FFa, FFb.

The first bottom plane BP1a, BP1b, BP1c may be arranged deeper within the retaining cavity 1260 relative to the second bottom plane BP2, see FIG. 13E.

At least two raised portions 1290a, 1290b may be arranged adjacent to the first bottom sub-portions BP1a and BP1b. Third bottom sub-portion BP1c may be arranged between portions BP1a and BP1b on a flexible support arm 1241. Flexible support arm 1241 may carry a supporting structure that is also arranged between the two raised portions 1290a, 1290b.

Alternatively, two separate flexible arms (each carrying only one respective rib directed radially outwards and only one rib directed radially inwards) may be used instead of flexible arm 1241, or alternatively only one arm, which may be smaller, with only one rib directed radially outwards and only one rib directed radially inwards.

An arrow 1248 indicates the proximal shift of the audible indicator 1300 during assembling, e.g. a shift of lateral tabs 1303a, 1303b from a pre-assembling position TP1a, TP1b to a post-assembling position TP2a, TP2b as described below in more detail.

Indicator holder 1250 may comprise along its middle axis from distal to proximal:
A ramp R2 having a comparably slight raising angle,
An intermediate portion IP having a surface parallel to bottom portion BP, e.g. parallel to bottom portion BP2,
A ramp R1 (corresponds to clicker rear support 1243) having a greater raising angle compared to the raising angle of ramp R2, and
A proximal portion PP having a surface parallel to bottom portion BP.

All these four regions are also illustrated in FIG. 13E within a cross section through the middle axis of indicator holder 1250. The function of each portion is described below in more detail.

Clicker mounting groove 1244 is illustrated on side wall SWb in FIG. 13B. However, there is a corresponding clicker mounting groove on sidewall SWa.

Non-coincident surfaces NCSFa1, NCSFa2, NCSFb1 and NCSFb2 are indicated by crosses. FIG. 13E illustrates the position of non-coincident surface NCSFb1, NCSFb2 relative to the cross section through the middle axis of indicator holder 1250. The function of these surfaces is described below in more detail.

The index “a” relates to the lower half of FIG. 13B. The index “b” relates to the upper half of FIG. 13B.

FIG. 13C illustrates a perspective view of a supporting structure on the distal end of a flexible support arm 1241.

Flexible support arm 1241 may be configured to support the audible indicator 1300 directly, i.e. by having physical contact. The flexible support arm 1241 may comprise at least one of the following features:
a) The flexible support arm 1241 may be arranged at least partially within the first bottom portion (plane) BP1c and may extend away from the second bottom portion (plane) BP2,
b) The flexible support arm 1241 may be configured to support the audible indicator 1300 onto a plunger 1000 of a drug delivery device 100, see also radially inward directed ribs 1241.2a and 1241.2b in FIG. 13C,
c) The flexible support arm 1241 may comprise an upwardly (radially outwardly) directed first supporting structure that comprises at least two supporting portions SPPa, SPPb that comprise supporting faces that are shaped complement to the angle between wing shaped sections 1301a, 1301b of the audible indicator 1300,
d) The flexible support arm 1241 may comprise a holding structure comprising at least one ramp or at least two ramps 1241.1a, 1241.1b,
e) wherein the flexible support arm 1241 may comprise a further or second supporting structure directed in an opposite direction (downwardly or radially inwardly) relative to the direction in which the first supporting structure extends from supporting arm 1241. The second supporting structure may comprise ramps 1241.2a and 1241.2b.

Supporting portions SPPa, SPPb may be arranged on a lateral wall portion WP, especially on the top of lateral wall WP. Wall portion WP may have a gable-like shape. An edge portion EP may be arranged at the connecting line of both supporting portions SPPa, SPPb. Edge portion EP may decline in the proximal direction P. The decline angle of edge portion EP may be adapted to the angle or may be equal to the angle of the second V in the second conformation state, see FIG. 13E, angle W2. Angle W2 may be in the range of 10 degrees to 20 degrees. Outwardly directed ramp-like element 1241.1 as described above, may comprise especially two ramps 1241.1a and 1241.1b arranged on both lateral ends of lateral wall WP.

Wall portion WP may be optional, especially if ramps 1241.1a and 1241.1a are adapted to angle W2. As described above, outwardly directed ramp-like element 1241.1 may comprise especially two ramps 1241.1a and 1241.1b. The index “a” relates to the foreground of FIG. 13C. The index “b” relates to the background of FIG. 13C.

Inwardly directed ramp-like element 1241.2 as described above, may comprise especially two ramps 1241.2a and 1241.2b. No further wall portion may be arranged between ramps 1241.2a and 1241.2b, e.g. in order to allow insertion of ramps 1241.2a and 1241.2b into proximal cutouts of plunger 1000. However, in other embodiments only one inwardly directed ramp 1241.2 may be used (e.g. on a position in the middle of ramps 1241.2a and 1241.2b as illustrated) or there may be a wall portion between ramps 1241.2a and 1241.2b, e.g. if plunger 1000 does not have proximal cutouts and/or if the proximal edge or plunger 1000 is used for triggering of audible indicator 1300, i.e. triggering happens when proximal end of plunger 1000 passes by on ramps 1241.2a and 1241.2b in distal direction D. Other modifications are possible as well.

A gap G1 may be located at three sides of support arm 1241 as illustrated, e.g. there may be three portions of gap G1: a first portion between bottom portion BP1a and arm 1241, a second portion between web portion 1291 and distal edge (free end) of support arm 1241 and a third portion between support arm 1241 and bottom portion BP1b.

On the fourth side of support arm 1241, there may be a groove G2, e.g. there may be thinner material compared to the thickness of material in bottom portion BP1c. Groove G2 may be located between proximal end (fixed end) of support arm 1241 and bottom portion BP2. Groove G2 may increase flexibility of support arm 1241. This means that no gap is located between support arm 1241 and bottom portion BP2.

FIG. 13D illustrates a perspective view of a guiding structure 1280a of the indicator holder 1250. In upper part of FIG. 13D, a view from below is shown onto the lower side of syringe support arm 1202 that carries indicator holder 1250, especially on assembling opening AOa. Audible indicator 1300 is not illustrated in the upper part of FIG. 13D, i.e. assembling opening AOa is still empty.

Lower part of FIG. 13D illustrates a zoomed in view into assembling opening AOa. Audible indicator 1300 is illustrated in the lower part of FIG. 13D, i.e. within assembling opening AOa, especially lateral tab 1303a on wing shaped section 1301a is visible.

Indicator holder 1250 may comprise a guiding structure 1280a that facilitates guiding of the respective lateral tab 1303a, 1303b from the auxiliary retaining space ARSa, ARSb into the final retaining space FRSa, FRSb. The guiding structure 1280a may be formed on a lower portion LPa, LPb of a rib 1270a, 1270b, for example, of a vertical rib 1270a, 1270b on the respective side wall portion SWPa, SWPb, see also FIG. 13B.

On the other side wall SWb, more specific on the other side wall portion SWPb there is also a vertical rib 1270b, comprising a lower portion LPb, see FIG. 13E and corresponding description as mentioned below. Lower portion LPb is similar to lower portion LPa, however, with mirror symmetry.

The lower portion of the rib 1270a, 1270b may extend beyond and/or from the first face FFa, FFb into the space between the final retaining space FRSa, FRSb and the auxiliary retaining space ARSa, ARSb. The lower portion LPa, LPb of the rib 1270a, 1270b may comprise a slanted face GFF1a that is slanted relative to the third face TFa, TFb and that is directed to the auxiliary retaining space ARSa, ARSb. The lower portion LPa, LPb of rib 1270a, 1270b may comprise a back stop face GFF2a at an opposite side of the slanted face GFF1a. The back stop face GFF2a may be configured to prevent movement of the respective lateral tab 1303a, 1303b from the final retaining space FRSa, FRSb back into the auxiliary retaining space ARSa, ARSb.

The lower portion LPa, LPb of the rib 1270a, 1270b may comprise an end face GFF5a that is arranged deeper within the retaining cavity 1260 than the first face FFa, FFb, especially nearer to the bottom portion BP1a, BP1b, BP1c compared to the adjacent portion of the first face FFa, FFb.

A face GFF3a is not visible in FIG. 13D but may correspond to the face of rib 1270a that directs into the retaining cavity 1260. A face (surface) GFF4a is directed into the opposite direction, e.g. away from retaining cavity 1260.

A deflection of the clicker tab 1301a in the downward direction (arrow Ar2) may allow it to clear, e.g. to bypass, the front-stop boss, e.g. vertical rib 1270a, 1270b respectively. Triangular portion 1331 of lateral tab 1301a may slide on slanted face GFF1a during shift of audible indicator 1300 and lateral tab 1303a, see arrow 1248 in FIGS. 13B and 13D.

Raised portion 1290a that is also illustrated in FIG. 13D, may guide connecting portion 1329 during the shift of audible indicator 1300.

FIG. 13E illustrates a cross section through the longitudinal axis of the indicator holder 1250. Indicator holder 1250 may comprise at least one guiding ramp R1, R2 arranged within the retaining cavity 1260 and configured to guide a front portion, for example, a front edge 1362, 1363 of the audible indicator 1300 during insertion of the at least two lateral tabs 1303a, 1303b into the final retaining spaces FRSa, FRSb thereby increasing a distance between a central part of the audible indicator 1300 and the bottom portion BP, especially second bottom portion BP2. Ramp R1 may be configured to guide a front edge of the plastically deformed bend 1302.

FIG. 13E illustrates two non-coincident surfaces NCSFb1 and NCSFb2. As mentioned above there are similar surfaces NCSFa1 and NCSFa2 near the other side wall SWa on which audible indicator 1300 is supported in conformation state S1, see angle W3 that is described below in more detail.

However, FIG. 13E illustrates audible indicator 1300 in the second conformation state S2, see dashed line and second bend, i.e. elastically (resilient, return to its original shape in conformation state S1) deformed bend 1350. In the second conformation state S2, audible indicator 1300 may be supported on edge portion EP, supporting portions SPPa (see FIG. 13C) and SPPb (see FIG. 13C), optionally on the lower side of elastically deformed bend 1350 on bottom portion BP2 as well as on ramp R1 but, for example, not on other locations. As mentioned above, the angle of supporting portions SPPa and SPPb may correspond to the angle between wing shaped sections 1301a and 1301b, for example, in both states S1 and S2. The angle between wing shaped sections 1301a and 1301b may not change or may only slightly change when audible indicator 1300 is primed, i.e. bended about axis C in order to provide the double V shape, wherein one V opens to the top and the other V opens to the bottom of retaining cavity 1260. This kind of double V structure is also known as a saddle structure.

FIG. 13E illustrates from the left to the right within retaining cavity 1260:
1) In the foreground: A web portion 1291 that laterally connects both raised portions 1290a, 1290b and is separated from supporting arm 1241 by gap G1,
1) In the background: a distal part D of a comparably high rib that does not have a reference sign and a distal part of raised portion 1290b.
2) In the foreground: supporting arm 1241.
2) In the background: a proximal half of the comparably high rib without reference sign, clicker mounting groove 1244 (indicated by brace in FIG. 13E), a proximal part of raised portion 1290b, auxiliary retaining space ARSb, vertical rib 1270b (including lower portion LPb), clicker tab restraint 1242 (all three relevant surfaces FFb, SFb and TFb are numbered by reference signs).
3) In the foreground: bottom portion BP2, ramp R2, intermediate portion IP, ramp R1 and proximal portion PP.
3) In the background: further portions of side wall SWb.

Moreover, FIG. 13E illustrates bottom portion (plane) BP1c, bottom portion BP2 and raised portion 1290b.

An angle W2 may be defined between lower surface of syringe support 1202 (alternatively second bottom plane/portion BP2) and edge portion EP. Angle W2 may be in the range of 10 degrees to 20 degrees.

An angle W3 may be defined between a connecting line connecting non-coincident surfaces (support points) NCSFb1 and NCSFb2 and a line parallel to second bottom plane/portion BP2 through non-coincident surfaces (support points) NCSFb1. Angle W2 may be in the range of 1 degree to 2 degrees, especially angle W3 may have a value of 1.25 degrees.

The following manufacturing steps may be used to assemble clicker 1300 (audible indicator) into indicator holder 1250:

A) Place Clicker 1300 onto Drive Spring Holder 1200

Clicker 1300 may bottom out on the drive spring holder 1200 with its lateral tabs 1303a and 1303b inside the corresponding cutouts, i.e. auxiliary retaining space ARSa, ARSb, see pre-assembling tab positions TP1a and TP1b in FIG. 13B.

The clicker 1300 may be supported on non-coincident surfaces NCSFb1 and NCSFb2, see FIGS. 13B and 13E and on corresponding non-coincident surfaces NCSFa1, NCSFa2, see FIG. 13B on the other side of retaining cavity 1260 near side wall SWa. There may be only one connection point on these non-coincident surfaces since there is the angle W3 and the clicker 1300 is tilted with regard to the planes formed by these surfaces.

Only one clicker 1300 may be assembled. Alternatively, two clickers 1300 may be assembled within each of the syringe support arms 1202.

B) Final Insertion of Clicker 1300 into Drive Spring Holder 1200

Pre-assembly step state: Clicker 1300 may already be aligned positional, e.g. axially and/or laterally, relative to retaining cavity 1260. Prior to this step, the clicker may be supported on these non-coincident surfaces NCSFa1, NCSFa2, NCSFb1 and NCSFb2 which lead to it being oriented at an angle of approximately 1.25° (degree).

The clicker 1300 may be pushed proximally into the drive spring holder 1200, see FIG. 13B, arrow 1248, e.g. manually or automatically. A special tool may be used or no tool may be used.

Both clicker tabs 1303a, 1303b may bottom out on the drive spring holder 1200 and reach their end positions within final retaining spaces FRSa, FRSb, see tab positions TP2a and TP2b in FIG. 13B.

The clicker 1300 may be held down (without permanent deformation) throughout this assembly step so that it does not lift up and out of position, e.g. its lateral and/or axial position with regard to retaining cavity 1260.

The two lateral tabs 1303a and 1303b of the clicker (audible indicator) 1300 may be pressed down relative to the main body of the clicker 1300, such that they bend front-stop boss (rib 1270a, 1270b) under the drive spring holder 1200 and avoid damaging it. This may be done without a special tool or by using a special tool, e.g. depending on the shape of the lateral tabs 1303a, 1303b. If triangular portions(s) 1331 are used, no special tool may be necessary for pressing down the two lateral tabs 1303a and 1303b, especially during the proximal shift of clicker 1300, see FIGS. 13B, 13D, arrow 1248.

FIG. 13F illustrates a rear sub-assembly RSA after assembling of the audible indicator 1300 but prior to priming of the audible indicator 1300.

A method of assembling an audible indicator 1300 into a retaining cavity 1260 of a body 700 may comprise at least one of the following:
Pre-positioning (see step A mentioned above) of the audible indicator 1300 within a retaining cavity 1260 adapted to retain the audible indicator, wherein at least two lateral tabs 1303a, 1303b of the audible indicator 1300 are positioned within respective auxiliary retaining spaces ARSa, ARSb that are arranged adjacent to the retaining cavity 1260,
Moving or axially (e.g. proximally) shifting (see step B mentioned above) the audible indicator 1300 within the retaining cavity 1260 thereby moving the at least two lateral tabs 1303a, 1303b from the auxiliary retaining spaces ARSa, ARSb to final retaining spaces FRSa, FRSb,
Using a priming tool 1292 (see FIG. 13H and corresponding description) in order to bring the audible indicator 1300 from a first conformation state S1 into a second conformation state S2 when the at least two lateral tabs 1303a, 1303b are positioned within the final retaining spaces FRSa, FRSb,
wherein the audible indicator 1300 may be biased to store energy in the second conformation state S2 in order to generate an audible signal when it is switched from state S2 to state S1 and is not biased in the first conformation state S1.

The audible indicator 1300 may be configured to generate an audible signal when changing from the second conformation state S2 to the first conformation state S1. Alternatively, a slightly biased state S1′ may be used instead of non-biased state S1.

A first dashed line DL1 indicates an exemplary position of a support plane of drive spring holder 1200 during priming. The priming position may be as illustrated in FIG. 13I, e.g. vertically orientated to longitudinal axis A of drive spring holder 1200. However, other priming positions are possible as well.

A first arrow Ar4 indicates a support force, i.e. counter force to a priming force. The priming force may be e.g. in the range of 35 N (newton) to 45 N or within other appropriate ranges.

A second dashed line (Datum line) DL2 may indicate a position of the beginning of a priming stroke (manually or automatically). A distance Di1 between both dashed lines DL1 and DL2 may e.g. be 20 mm (millimeter).

A second arrow Ar6 may indicate a noise (i.e. there may be generated a noise/signal during priming) priming stroke from the position indicated by second dashed line DL2, e.g. a priming stroke greater than or equal to 7.75 mm (millimeter).

The clicker 1300 may be pushed in or into the notched area, e.g. in the notch 1304, using a priming tool, see e.g. priming tool 1292 as illustrated in FIG. 13H.

The clicker 1300 may be held in a primed state without application of external forces, e.g. because of support by plunger 1000 and clicker support arm 1241. The clicker 1300 may remain in the primed stated until the rear sub-assembly (RSA) is inserted into the front sub-assembly (FSA), see section 15 below.

A distance Di2 may be much greater than a distance Di3, e.g. at least 10 percent greater than distance Di3 or at least 20 percent greater. Distance Di2 is measured between central axis A of drug delivery device 100 (corresponds to central axis of plunger 1000) and a middle plane of syringe support arm 1202 (with audible indicator 1300, i.e. upper syringe support arm 1202 in FIG. 13F). Distance Di3 is measured between central axis A of drug delivery device 100 (corresponds to central axis of plunger 1000) and a middle plane of syringe support arm 1202 (without audible indicator 1300, i.e. lower syringe support arm 1202 in FIG. 13F) in an unbiased condition, e.g. in a relaxed or normal position. Syringe support arm 1202 that comprises clicker 1300 is in a splayed condition because lower ramps 1241.2a, 1241.2b are arranged on the outer circumference of plunger 1000 and supporting portions SPPa and SPPb are in contact with clicker 1300 that is fixed within syringe support arm 1202 by the lateral tabs 1303a, 1303b arranged within final retaining spaces FRSa, FRSb. In this state clicker 1300 may still be comparably flat, i.e. only single V shape as clicker 1300 is in state S1 and not yet primed contrary to the “double V shape” in stated S2.

Syringe support arms 1202 of drive spring holder 1200 may be splayed out during insertion of plunger 1000 into drive spring holder 1200. If this is not done then the drive spring holder could be damaged. The maximum distance that the ends of the legs 1202 may be splayed out may be e.g. at least 5 mm, e.g. 10 mm from their original position.

FIG. 13G illustrates the rear sub-assembly (RSA) after priming of the audible indicator 1300, for example, using a priming tool 1292, see FIG. 13H. Syringe support arm 1202 that carries clicker 1300 is again in its normal or non-splayed (biased) condition as clicker 1300 is primed and has a double V shape. The double V shape in state S2 may allow distal part of clicker 1300 to apply much lower force or even only a very small force onto flexible arm 1241, e.g. via supporting portions SPPa and SPPb. Moreover, in the double V shape in state S2, the proximal part of clicker 1300 may rest on rear support 1243. Alternatively, in state S2, the proximal part of clicker 1300 may not rest on rear support 1243 but may be arranged with a distance greater 0 mm or greater 1 mm to rear support 1243. Lateral tabs 1303a and 1303b may still be arranged within final retaining spaces FRSa, FRSb. However, due to the double V shape, there may be no constraint force any more that is applied via clicker 1300 to syringe support arm 1241.

FIG. 13H illustrates the priming tool 1292. The geometry of a tip portion 1294 of the priming tool 1292 may be relevant for secure priming, e.g. the tip portion 1294 of priming tool 1292 may correspond or may be complementary to the notch 1304. The shape of the tip portion 1294 priming tool 1292 may be important and proportions may be as illustrated.

Priming tool 1292 may comprise the tip portion 1294 and a tip holder 1296. Tip portion 1294 may have a radius Ra1 in the range of 0.03 mm to 0.07 mm, e.g. a radius Ra1 of 0.05 mm (millimeter). A second radius Ra2 may be in the range of 0.07 mm to 0.13 mm, e.g. radius Ra2 of 0.1 mm (millimeter). Radius Ra2 may be located at the opening of the notch during priming of audible indicator 1300.

An angle W4 of the conically shaped tip portion 1294 may be in the range of 25 degrees to 35 degrees. Angle W4 may be defined between a central axis AX of tip portion 1294 and the outer surface of the cone, e.g. as illustrated in FIG. 13H.

A distance Di4 may correspond to the diameter of a cylindrical portion 1295 of tool 1292, e.g. in the range of 0.7 mm to 1.3 mm, e.g. 1 mm. The cylindrical portion may be arranged between tip portion 1294 and tip holder 1296.

Other priming tools may be used as well, e.g. depending on the shape of notch 1304 or other features used to ease priming of audible indicator 1300.

FIG. 13I illustrates a state during final assembling of the rear sub-assembly RSA and of a front sub-assembly FSA shortly before priming of the audible indicator 1300.

The priming tool, e.g. 1292, may strike the clicker 1300 at the point as defined by a distance Di5, e.g. at the notch area or notch 1304. This strike is indicated by an arrow 1299. Distance Di5 may be in the range of 25 mm to 35 mm, e.g. 30.5 mm plus or minus 0.2 mm. Thus, priming may be performed as a part of the final assembling of a device, especially of a drug delivery device 100.

### 14. Syringe Holder and Pre-Filled Syringe Assembly to the Device (FIGS. 14A to 14K)

As mentioned and shown in FIGS. 8A to 8D in specific embodiments the drug delivery device may comprise a container holder, e.g. a syringe holder 800 to allow an accurate support of the medicament container, e.g. the pre-filled syringe 900 during and after assembling. The syringe holder 800 may be adapted to assemble and hold the pre-filled syringe 900 within the device body 700 as will be further explained with respect to FIGS. 14A to 14K.

In one embodiment, the syringe holder 800 and the pre-filled syringe 900 may be assembled to the drug delivery device 100, after at least one or more other features had already been assembled thereto. For example, before assembling the syringe holder 800 and the pre-filled syringe 900 to the drug delivery device 100, e.g., to the device body 700, the needle cover spring 1100 and needle cover 500 may have already been inserted into the device body 700. Further, the grabber 400 may have been inserted into the cap 500 and the cap 500 with the inserted grabber 500 may have been connected to the needle cover 500 and assembled to the device body 700, as described in the foregoing.

The needle cover 500 and the needle cover spring 600 are not shown in FIGS. 14A to 14K to increase the legibility of the figures with respect to the assembly of the syringe holder 800 and the pre-filled syringe 900.

The assembly steps mentioned in the following, relate to the assembly of the syringe holder 800 and the pre-filled syringe 900. The terms first, second, etc., are not to be understood as related to the order of the assembly steps in relation to the assembly of the drug delivery device as a whole. The syringe holder shown in the following FIGS. 14A to 14K may be the syringe holder 800 described with respect to FIGS. 8A to 8D.

In a first assembly step, the syringe holder 800 may be inserted distally into the device body 700, e.g. through the proximal aperture 730 of the body 700. During the distal movement, the syringe holder 800 may be supported by the central support structure 701 of the device body 700.

To assist the insertion, the syringe holder 800 may comprise guiding features 811 on the edges between the rounded sections 813 of the holder flange portion 805 and the recessed sections of the holder flange portion 805 (see FIGS. 8A, 8B and 8D).

Additionally, the longitudinal rib 807 and the stop feature 809 at the distal end of the rib 807 may be used for positioning and optionally guiding, the syringe holder 800 axially with respect to the device body 700.

As the syringe holder 800 is inserted axially, e.g., distally, into the device body 700 from a proximal end of the device body 700, e.g. through the proximal aperture 730, the guiding features 811 may interact with the holder guiding ribs 726, thereby maintaining the desired orientation of the syringe holder 800 relative to the device body 700. The syringe holder 800 is moved distally relative to the device body 700, until the holding clips 806 align with the proximal cut-outs 714 of the device body 700.

As described in further detail in section 8 above, a proximal end of the holding clips 806 may be radially outwardly directed. Further the proximal end of the holding clips 806 may have an inclined surface which is radially outwardly inclined in the proximal direction P (see FIG. 8A). When the syringe holder 800 is moved distally inside the device body 700, the inclined surfaces come contact the edge 732 of the aperture 730 and cause a radial inward deflection of the flexible portions of the holding clips 806. Thus, the holding clips 806 are tensioned. Upon alignment of the proximal ends of the holding clips 806 with the proximal cut-outs 714, the proximal ends are free to move radially outwardly. Due to the tensioned flexible portions the proximal ends of the holding clips 806 move radially outwardly into the spaces provided by the proximal cut-outs 714. Thus, the syringe holder 800 is secured to the device body 700 in the first syringe holder position (first container holder position). In this position, the syringe holder 800 is secured to the device body 700 against a proximal movement relative to the device body 700. Further, in the first syringe holder position, the syringe holder 800 is secured to the device body 700 against relative rotational movement, e.g. by the holding clips 806 interacting with the proximal cut-outs 714 as well as by the guiding features 811 interacting with the holder guide ribs 726.

In a second assembly step, the pre-filled syringe 900 is inserted distally into the syringe holder 800 as schematically shown in FIG. 14A. Features of the drug delivery device 100 seen in this figure are not limiting with respect to the assembly step, except if explicitly stated so.

FIGS. 14B to 14F show a detailed view of the interaction between the syringe holder 800 and the pre-filled syringe 900 during assembly. In more detail, FIGS. 14B to 14F show the interaction between the flexible holder arms 801 and the pre-filled syringe 900, e.g. between the flexible holder arms 801 and the needle shield 914 and/or the shoulder 904, during the second assembly step.

In FIG. 14B, the pre-filled syringe 900 is inserted into the syringe holder 800. The syringe holder 800 may be in the first syringe holder position, as described before. As shown, in this stage of the second assembly step, the flexible holder arms 801 are in their relaxed state and the holder protrusions 803 (see FIG. 14E) protrude radially inwardly.

When the pre-filled syringe 900 is moved further distally relative to the syringe holder 800, as shown in FIG. 14C the distal end surface 914A of the needle shield 914 is brought into contact with, e.g. abuts on, the holder protrusions 803 of the syringe holder 800. This abutment represents a first tactile feedback indicating that the needle shield 914 has reached the holder protrusions 803.

The ramps on the holder protrusions 803 interact with the distal end surface 914A of the needle shield, thereby allowing the distal end surface 914A to outwardly deflect and tension the flexible arms 801 when the syringe 900 moves further distally relative to the syringe holder 800. Due to the deflection, the distal end surface 914A of the needle shield 914 is free to pass the holder protrusions 803 and the syringe 900 may be moved further distally relative to the syringe holder 800, as shown in FIG. 14D.

Due to their flexibility and the tension caused by the radial outward deflection, the holder arms 801, e.g., the holder protrusions 803, may be in continuous contact with the needle shield 914 during further insertion of the pre-filled syringe 900 into the syringe holder 800.

As shown in FIG. 14E, during the second assembly step, the pre-filled syringe 900 may move further distally relative to the syringe holder 800, until the needle shield 914 has fully passed the holder protrusions 803. Now, as shown in FIG. 14E, the flexible holder arms 801 are free to return to their relaxed, non-deflected, state. In other words, the flexible holder arms 801 may move radially inwardly and the holder protrusions 803 may snap into the space between the proximal end of the needle shield 914 and the shoulder 904 of the pre-filled syringe 900, thereby securing the pre-filled syringe 900 to the syringe holder 800.

The snap-in and impact of the holder protrusions 803 onto the pre-filled syringe 900, may represent a further tactile feedback during assembly, indicating that the needle shield 914 has passed the flexible holder arms 801.

As shown in FIG. 14F, during the second assembly step, the syringe 900 may be moved further distally relative to the syringe holder 800 such that the shoulder 904 of the barrel 902 may cause a radial outward deflection of the flexible holder arms 801 by overcoming the snap-in force of the flexible holder arms 801 acting on the shoulder 904. Hence, again, the flexible holder arms 801 may be tensioned due to an radial outward deflection caused by the shoulder 904 interaction with the holder protrusions 803. As the holder arms 801 move radially outwardly, the holder protrusions 803 move radially outwardly, as well, thereby disengaging from the shoulder 904 and allowing a further distal movement of the syringe 900 relative to the syringe holder 800. The distal movement may cause a separation of the holder protrusions 803 from a proximal end of the needle shield 914. The pre-filled syringe 900 may be moved further distally until the holder protrusions 803 are spaced apart from the proximal end of the needle shield 914 by a predetermined distance, e.g. distance D7. During the distal movement of the syringe 900, the holder protrusions 803 slide along an outer surface of the barrel 902, thereby pressing on the barrel 902 due to the tension of flexible holder arms 801. When the predetermined distance is reached, the protrusions 803 interacting with the outer surface of the barrel 902 may hold the syringe 900 in position relative to the syringe holder 800, due to a friction force (see FIG. 14F).

During the whole of the second assembly step, the syringe holder 800 may be kept in its first engaged position, i.e. the first syringe holder position by the engagement of the holding clips 806, with the proximal cut-outs 714 of the device body 700. The movement of the pre-filled syringe 900 through the syringe holder 800 may hence not cause a movement of the syringe holder 800 relative to the device body 700.

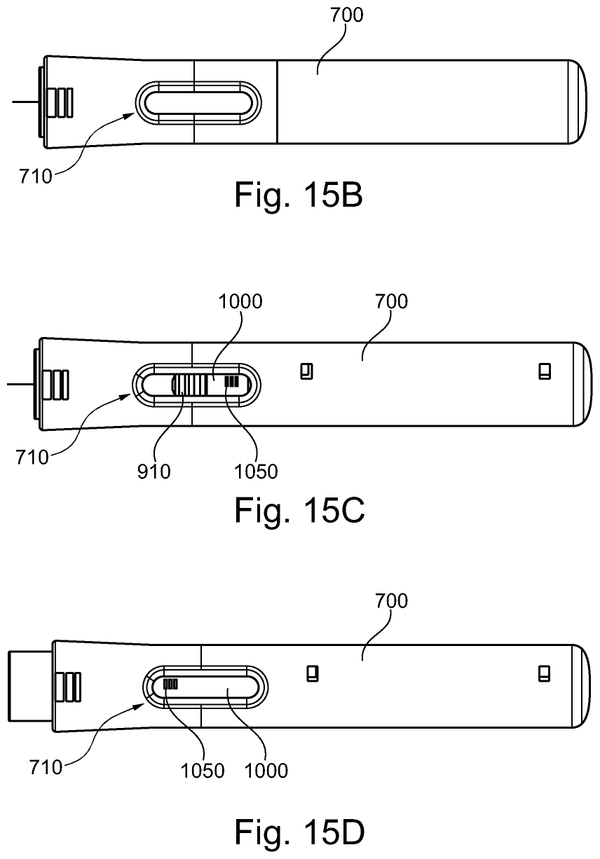

After the pre-filled syringe 900 is positioned in the syringe holder 800, the syringe holder 800 may be moved further distally into the device body 700 until it reaches a second syringe holder position (second container holder position), illustrated in FIG. 15C. In detail, due to the inclination of surfaces of the proximal ends of the holding clips 806, as described before, the syringe holder 800 is releasable from the device body 700 in its first syringe holder position. In detail, if an appropriate force in the distal direction D is applied to the syringe holder 800 being in the first syringe holder position, the inclined surfaces of the holding clips 806 are pressed against proximal surfaces of the proximal cut-outs 714. In a similar fashion as described before, when the syringe holder 800 is inserted through the aperture 730, the contact between the inclined surfaces and the proximal cut-outs 714 causes the flexible portions of the holding clips 806 to deflect radially inwardly, thereby disengaging from the proximal cut-outs 714. In other words, the syringe holder 800 is disengaged from the device body 700 and is free to move to a second syringe holder position (second container position), as described e.g. in section 7 above. FIG. 14G shows the position of the pre-filled syringe 900 and the syringe holder 800 when the syringe holder 800 is disengaging from the device body 700 after the second assembly step.

In FIG. 14H an assembling equipment in the form of a press-head 1400 is shown. The press head 1400 can be used for the next assembly step, described in the following figures.

Press head 1400 is formed as a substantially cylindrical element configured to be inserted into the device body 700 through the proximal aperture 730 and to be able to move axially along, e.g. distally through, the device body 700.

FIG. 14H shows a cross-sectional view of a possible embodiment of the press head 1400 used for the assembly process.

The press head 1400 comprises a body 1401 having a substantially cylindrical form.

The press head body 1401 comprises a substantially flat proximal surface 1410, which is orientated towards the proximal end of the device body 700, e.g. the proximal aperture 730, during use of the press head 1400 in the assembly of the drug delivery device 100.

On its distal surface 1420 the press head body 1401 comprises a recess 1430, formed e.g. as a flat cut through the press head body 1401. A diameter of the recess 1430 may correspond to the diameter of the syringe flange 912 such as to permit an insertion of the syringe flange 912 into the recess 1430 (e.g. FIGS. 14 J and 14 K).

As such the press head body 1401 comprises axially extending protrusions 1440, e.g. distally extending arms, configured such as to interact with a proximal surface of the syringe holder flange 804 during insertion of the press head 1400 into the device body 700 through the proximal aperture 730 of the device body 700.

The axial extension of the protrusions 1440 of the press-head 1400 distally beyond a bottom of the recess may correspond to a length D8. In one embodiment, the axial extension of the protrusions 1440, D8, may correspond to the distance, D7, between the distal end of the flexible holder arms 801 and the proximal end of the needle shield 914 of the pre-filled syringe 900 at the end of the second assembly step, as shown in FIG. 14F.

FIG. 14| shows a first sub-step of a third assembly step as well as a detailed view of the interaction between the pre-filled syringe 900 and a grabber 400 positioned in a cap 200 (see e.g. FIGS. 3A to 3I and 4A to 4H for a description of possible grabbers and caps and their respective interaction).

The syringe holder 800 and the pre-filled syringe 900 may be pushed distally further inside the device body 700 for example by the press head 1400, when the press head 1400 is inserted into the device body 700 through the aperture 730. The press head 1400 may interact with the syringe holder 800 through the contact of the protrusions 1440 and the syringe holder 800. This may correspond to a first situation. The press-head 1400 may transmit an axial force, e.g. a distally directed force, on the syringe holder 800 in order to push the syringe holder 800 further distal relative to the device body 700.

The flexible holder arms 801 and, for example, the protrusions 803 of the flexible holder arms 801 may be configured such as to exercise a force, e.g. a friction force, on the barrel 902 of the pre-filled syringe 900. The friction force may be strong enough to secure the pre-filled syringe 900 within the syringe holder 800 against relative axial and/or rotational movement, when the syringe holder 800 is pushed distally along the body 700. In other words, the interaction between the press-head 1400 and the syringe holder 800 is sufficient to move the pre-filled syringe 900 in the distal direction together relative to the device body 700.

As shown in FIG. 14I during the first sub-step of the third assembly step, the syringe holder 800 may be pushed axially in the distal direction until the rigid needle shield 914, e.g. the distal end surface 914A thereof, contacts the barbs 412 of the grabber 400, when the grabber is assembled to the cap 200 and the cap is assembled to the device body, as described in the foregoing sections. The barbs may therefore act as a deformable engagement structure for the pre-filled syringe, e.g., for the distal end of the pre-filled syringe.

The barbs 412, as explained in more detail in the parts of the disclosure relating to the grabber (e.g. FIG. 4A to 4H), are flexible and biased radially inwardly, thereby forming a first obstruction for the pre-filled needle 900 against further distal movement. The contact between the pre-filled syringe 900, e.g. the needle shield 914, with the barbs 412 causes a tactile feedback, indicating that the finishing position for the first sub-step of the third assembly step is reached.

FIG. 14J shows a second sub-step of the third assembly step as well as a detailed view of the interaction between the pre-filled syringe 900 and the grabber 400 positioned in the cap 200.

In the second sub-step of the third assembly step, the press head 1400 may be further pushed axially, e.g. in the distal direction, into the device body 700 causing a distal movement of the syringe holder 800 relative to the device body 700, through the interaction between the protrusions 1440 of the press head 1400 and the proximal surface of the syringe holder flange 804. The pre-filled syringe 900 may however be prevented from being pushed in the distal direction relative to the device body 700, by the interaction of the distal surface 914A of the needle shield 914 and the inwardly bent barbs 412. Specifically, the reactive force of the barbs 412 acting on the needle shield 914 is strong enough to prevent an axial movement of the pre-filled syringe 900 in the distal direction, when the syringe holder 800 moves distally during the second sub-step of the third assembly step. In other words, as the force of the flexible holder arms 801 acting on the barrel 902 may be weaker than the reactive force of the barbs 412 acting on the needle shield 914, the syringe holder 800 may move distally relative to the pre-filled syringe 900. In other word the engagement force between the pre-filled syringe and the syringe holder, however, may be not as strong as the retaining force, e.g. the reactive force, of the barbs on the pre-filled syringe, e.g. on the distal end of the pre-filled syringe. As such, when the pre-filled syringe engages the barbs it is retained, and a distal movement of the syringe holder results only in a distal movement of the syringe holder with respect to the device body and the pre-filled syringe, without moving the pre-filled syringe. In other words the syringe holder moved with a force being less than an engagement force between the medicament container and the deformable engagement structure, such as to move only the container holder in the distal direction

The force with which the syringe holder and the re-filled syringe may be moved distally relative to the barbs into an end position is greater than an engagement force between the barbs and the pre-filled syringe. So, it becomes possible to overcome the engagement forces acting on the pre-filed syringe by the barbs, which counteracts the movement of the pre-filled syringe towards the end position.

The second sub-step of the third assembly step may be concluded when the movement of the syringe holder 800 in the distal direction relative to the pre-filled syringe 900 and/or the device body 700, caused by the press head 1400, causes the flexible holder arms 801, e.g., the holder protrusions 803, to snap back into the space between the proximal end of the needle shield 914 and the shoulder 904 when the protrusions 803 pass the shoulder 904 during the distal movement of the syringe holder 800 relative to the pre-filled syringe 900. At the end of this movement, when the protrusions 803 have snapped back into the space between the proximal end of the needle shield 914 and the shoulder 904, the pre-filled syringe 900 may be in its final position relative to the syringe holder 800.

In a fourth assembly step the press head 1400 is further moved axially, e.g. in the distal direction. This may cause a corresponding movement of the syringe holder 800 and the pre-filled syringe 900 inside the device body 700 until the syringe holder 800 reaches a second syringe holder position, e.g. a second container holder position, in the device body 700. During this movement, this force of the barbs 412 may be overcome and the needle shield 914 may move distally relative to the grabber 400 to its final position inside the grabber 400.

The interaction between the press head 1400, the syringe holder 800 and the pre-filled syringe 900 may however vary depending on the pre-filled syringe 900 that is used.

In one possible embodiment, for example when the pre-filled syringe 900 is a BD (Becton Dickinson) Neopak 1 ml long pre-filled syringe (with a 27 gauge special-thin-walled needle), the press head 1400 may be in contact with the syringe holder 800, e.g. through the protrusions 1440, but not with the pre-filled syringe 900. The distal flat portion of the flexible holder arms 801 may contact the proximal end of the needle shield 914, such that a movement of the syringe holder 800 causes a movement of the pre-filled syringe 900 as well. When the press head 1400 is pressed axially in distal direction, it pushes the syringe holder 800 in distal direction thereby also pushing the pre-filled syringe 900 in distal direction.

In another possible embodiment, for example when the pre filled syringe is an Ompi EZ-Fill 1 ml long pre-filled syringe (with a 27 gauge thin-walled needle), the press head 1400 may also contact, e.g. abut on, the flange of the pre-filled syringe 900, such that when the press head 1400 may be pushed in distal direction it may push both the syringe holder 800 and the pre-filled syringe 900 in the distal direction.

For example, the pre-filled syringe flange 901 may abut on a distal surface of the press head 1400 in the recess 1430 when the protrusions 1440 contact the syringe holder. This may for example be when the axial extension of the protrusions 1440 of the press head 1400, i.e. D8 (see FIG. 14H), corresponds to the distance D7 between the distal end of the flexible holder arms 801 and the proximal end of the needle shield 914 of the pre-filled syringe 900 (see e.g. FIG. 14F). In other words, the protrusion 1440 of the press head 1400 may therefore abut on the proximal end 804 of the syringe holder 800, while the syringe flange 912 of the pre-filled needle 900 may be received into the recess 1430 of the press head 1400 and abuts the press head 1400. This may correspond to a second situation. A further movement of the press head 1400 into the distal direction therefore may cause a simultaneous movement of both the syringe holder 800 and the pre-filled syringe 900.

The disclosure, however, is not limited to specific types of syringes and could also be applied, for example, when the pre-filled syringe 900 is a BD (Becton Dickinson) Neopak 1 ml long pre-filled syringe (with a 27 gauge special-thin-walled needle).

In both scenarios, the press head may be pressed distally until the needle shield 914 is fully inserted into the grabber 400. The holding clips 806 align with distal slots, e.g. the distal cut-outs 713 of the device body 700. Upon alignment, the holding clip 806 interact with the cut-outs 713 by deflecting radially outwardly into the space formed by the cut-outs 713. This is the second engaged position of the syringe holder 800 (i.e. the second container holder position). The interaction between the holding clips 806 and the cut-outs 713 may limit, for example, prevent, a proximal movement and/or a rotational movement of the syringe holder 800 relative to the device body 700.

In its final position in the device body 700, the holder arms 801 are moved radially inwardly and restrained by a narrowing tube, e.g. the conical inner surface 703.1, within the device body 700 to ensure that the holder arms 801 locate correctly on the syringe shoulder 904 and to maintain this position.

In the final assembled state, the pre-filled syringe 900 is biased proximally relative to the syringe holder 800. This may ensure that the syringe holder arms 801 locate on the syringe shoulder 904 and support the pre-filled syringe 900 correctly. Specifically, the flexible holder arms 801 in the final assembled position are positioned between the axial support front end 703 and the shoulder 904 of the pre-filled syringe 900, thereby avoiding a distal movement of the shoulder 904 beyond the axial support front end 703 (see e.g. FIG. 7D).

### 15. Feedbacks (FIG. 15A to 15D)

FIG. 15A shows a flow diagram of an exemplary feedback order during use of the drug delivery device. FIGS. 15B to 15D show several situations in which different elements of the drug delivery device are visible through a drug window.

In order to facilitate the usage of the drug delivery device for a user, e.g. for a patient, several feedback mechanisms indicating several steps of the injection process may be implemented. The feedbacks may involve tactile, audible, and/or visible feedbacks.

A first feedback F1, e.g. a start of dose dispensing feedback F1, may indicate to the user that the dispensing of a drug dose has commenced.

The first feedback system may for example be a tactile and/or audible feedback mechanism caused by the impact of the plunger shaft 1010 (often also called plunger rod) with the plunger stopper 910 once the plunger 1000 is released (see e.g. in sections 5, 10 and 12). The impact may cause a vibration of the drug delivery device 100, e.g., vibrations on the device body 700 which a user, holding the device in hand, may feel. The vibration may indicate the start of the drug dispensing process.

The impact of the plunger shaft 1010 on the plunger stopper 910 may additionally or alternatively cause an audible feedback hearable by the user and indicating the start of the drug dispensing process.

Additionally, or alternatively, the first feedback F1 may also comprise a tactile and/or audible feedback caused by the impact of the plunger with the drive spring holder 1200 when the plunger is released by the plunger release mechanism 1025. As described above with reference to the plunger, the plunger may be rotationally biased before release by the release mechanism. Once the plunger is released it rotates and moves distally. The rotation may optionally stop when the plunger impacts an element of the drive spring holder 1200. The impact then causes the tactile and/or audible feedback.

Before initiating the dose dispensing process, the plunger shaft 1010 and the plunger stopper 910 may not be visible through the drug window 710 of the device body 700. This may correspond to a first state of the drug delivery device 100 shown in FIG. 15B.

During the in injection process a second feedback F2, e.g. an injection process feedback F2, may be provided. The second feedback F2 may be a visible feedback, e.g. of the plunger shaft 1010 and/or the plunger stopper 910 moving distally along the drug window 710. The second feedback F2 may indicate that the drug Dr is being expelled, as for example shown in FIG. 15C. this may correspond to an intermediate state.

The plunger stopper 910 and the plunger 1000 may comprise different colors which may aid the user in identifying the two different features and observing a movement relative to the device body 700 though the drug window 710 (see e.g. FIG. 15C). In some examples, the different colors may help in the identification of the border between the plunger shaft 1010 and the plunger stopper 910.

During the dose dispensing process there is a state in which the plunger stopper 910 disappears from the drug window 710 and the plunger shaft 1010 fills the whole drug window 710 (similarly as in FIG. 15D), e.g. a second state. The plunger shaft 1010 however may be still moving in a distal direction as the dose dispensing process may not be yet finalized, e.g. in a first sub-state of the second state. In such a state of the drug delivery device, it may be difficult for a user to clearly recognize if the plunger shaft 1010 is still moving and the dose dispensing process is in progress or if the plunger shaft 1010 is still and the dose dispensing process has finalized.

As can be seen and as described above with respect to the plunger shaft 1010 the plunger shaft may comprise feedback elements 1050, e.g. auxiliary structure, for example in the form of a visually identifiable feature, which may be read by a reading device. In FIGS. 15C and 15D the feedback element comprises three grooves 1050 arranged on a distal portion of the plunger shaft 1010 as described above with respect to the plunger shaft 1010. The feedback elements 1050 aid the user to understand if the plunger shaft 1010 is still moving in a distal direction or not.

At the end of the injection process at least one further feedback F3 is provided.

A third feedback F3, e.g. an end of injection feedback F3, may be a tactile and/or audible feedback caused by the clicker 1300 providing a sound, as described above, e.g. in section 13).

Additionally, or alternatively, the end of injection feedback F3 may also comprise a visible feedback.

For example, as shown in FIG. 15D, the plunger shaft 1010 may be configured such that at the end of the dose dispensing process, the at least one groove 1050 may be arranged on a distal portion, e.g. on the most distal end, of the drug window 710. This may correspond to a second sub-state of the second state. This and additionally or alternatively the fact that the grooves 1050 may show that the plunger shaft 1010 does not move distally, may be an indication that the dose dispensing process has finished.

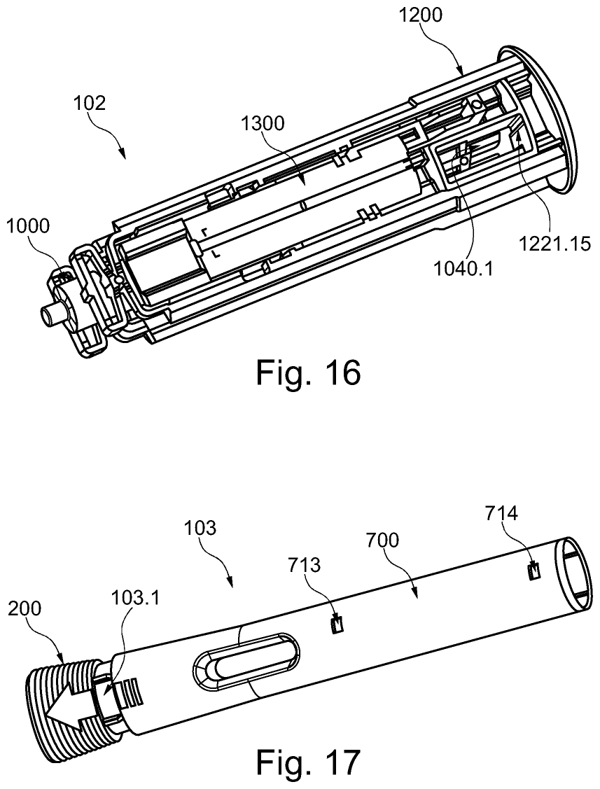

### 16. Assembly of the Device (FIGS. 16 and 17)

FIGS. 16 and 17 show a rear sub-assembly 102 (RSA) and a front sub-assembly 103 (FSA) of the drug delivery device 100. The drug delivery device 100 is designed to be assembled using these two sub-assemblies 102 and 103 which can be manufactured at a location separate from final assembly, providing a flexible supply chain.

FIG. 16 shows the rear sub-assembly 102, which includes the plunger 1000, the drive spring 1100 (not shown), the drive spring holder 1200, and the clicker 1300. The drive spring holder 1200 forms a supporting element of the rear sub-assembly 102. To assemble the rear sub-assembly 102, the clicker 1300 is inserted into the drive spring holder 1200. The plunger 1000 and the drive spring 1100 are then inserted into the drive spring holder 1200, compressing the drive spring 1100. As described above, a rotation of the plunger 1000 relative to the drive spring holder 1200 occurs which locks the plunger 1000 in the drive spring holder 1200 and thus holds the drive spring 1100 in the compressed state. The assembly of the rear sub-assembly 102 is primarily axial with the only exceptions of inserting the clicker 1300 into the drive spring holder 1200. The rear sub-assembly 102 includes a mechanism to prevent accidental unlocking of the plunger 1000 in the drive spring holder 1200. For this purpose, as described above, the drive spring holder 1200 has the indent 1221.15 which cooperates with the first plunger boss 1040.1 to reduce the risk of the locking between the plunger 1000 and the drive spring holder 1200 being released when the rear sub-assembly 102 falls on the tip of the plunger 1000.

FIG. 17 shows the front sub-assembly 103, which includes the needle cover 500 (not shown), the needle cover spring 600 (not shown), the device body 700, the cap 200 and the grabber 400 (not shown).

The assembly of the front sub-assembly 103 is entirely axial. To assemble the front sub-assembly 103, first the needle cover spring 600 and then the needle cover 500 is inserted into the distal end of the device body 700.

Here, the needle cover 500 is pushed in the distal direction along the longitudinal axis of the device body 700 until the cuboidal elevations 510.1 of the flexible arms 510 engage in the needle cover locking elements 720.

Then, as described above, the grabber 400 is inserted into the cap 200 and the two are then mounted on the needle cover 500.

After this step, the syringe 900 or the syringe holder 800 and the syringe 900 are inserted into the device body 700, with the holding clips 806 of the syringe holder 800 engaging in the distal cut-outs 713 of the device body 700 in order to prevent a relative movement of the device body 700 relative to the syringe holder 800, for example, permanently.

As seen in FIG. 17, the cap 200 is held slightly apart from the device body 700 when the front sub-assembly 103 is assembled. In other words, in its fully assembled state, the front sub-assembly 103 has a cap-gap 103.1 in the axial direction between the distal end portion of the device body 700 and the proximal end portion of the gripping surface 202. As shown in FIG. 17, the length of the cap-gap 103.1 may correspond to the length of the anti-rotation ribs 205 in the axial direction. Additionally, or alternatively, the length of the cap-gap 103.1 can correspond to the distance between the proximal end portion of the gripping surface 202 and a distal recess end 213.1 (see FIGS. 3H and 3I). In these contexts, the term corresponding means that the length can be the same. Furthermore, the cap-gap 103.1 can have a length that allows the area of the cap between the distal recess end 213.1 and a proximal recess end 213.2 to be completely encased by the device body 700. In other words, for example, the cap-gap 103.1 may have a length that allows the portion of the cap between the distal recess end 213.1 and the proximal recess end 213.2 to be fully slid into the device body 700. The cap recesses 213 help the flexible arms 510, e.g., the web 510.2, not to be pressed radially inwards in the direction of the longitudinal axis by the inner peripheral surface of the cap 200 as long as the cap-gap 103.1 between the cap 200 and the device body 700 exists.

The cap recesses 213 can be designed such that their length in the axial direction corresponds to the length of the flexible arms 510 or the web 510.2 in the axial direction. The length of the flexible arms 510 in this context can be the distance between the distal end of the circular recesses 510.3 and the proximal end of the cuboidal elevation 510.1. Consequently, the flexible arms 510 may remain stretched out in the needle cover locking elements 720 (see the third cover position Z) as long as the cap-gap 103.1 between the cap 200 and the device body 700 exists. Thus, since the front sub-assembly 103 is manufactured and stored separately from the rear sub-assembly 102, fatigue of the elasticity of the elastic arms 510 made of plastic, which would occur when the flexible arms 510 are kept deflected radially inward (see first and second cover position X and Y) for a long period of time, can be prevented. A storage of the front sub-assembly 103 with the cap-gap 103.1 thus helps to ensure the subsequent functional reliability of the drug delivery device 100 even when the two subassemblies 102 and 103 are stored for a longer period of time.

The final assembly of the drug delivery device 100 is accomplished by placing the pre-filled syringe 900 into the syringe holder 800 (if present) and then sliding both into their final position in the device body 700 as described above. In doing so, the syringe 900 is biased rearwardly relative to the syringe holder 800 to ensure that the syringe holder arms 801 rest on the syringe shoulder 904 and properly support the syringe 900. The holding clips 806 return to the distal cut-out 713 in their unstressed condition. The clicker 1300 is then biased by applying a force to the notch 1304 and the rear sub-assembly 102 is then inserted into the device body 700 until the flexible part 1203.1 of the snap arms 1203 of the drive spring holder 1200 engages the proximal cut-outs 714 of the device body 700.

In order to prime the device 100, the needle cover 500 may be moved from the third cover position Z to the first cover position X, among other things. For this purpose, the cap-gap 103.1 between the cap 200 and the housing 700 is closed by the cap 200 being moved in the proximal direction relative to the housing 700 until the proximal end region of the gripping surface 202 hits the distal opening of the device body 700. This may cause the distal recess ends 213.1, to contact the ramps of the webs 510.2 at the distal end of the webs of the flexible arms 510, sliding over the webs 510.2 and thereby forcing the flexible arms 510 radially inward, releasing the lock between the flexible arms 510 and the needle cover locking elements 720. By releasing this connection, the needle cover 500 can now be moved in the proximal direction relative to the device body 700 into first cover position X. This movement of the needle cover 500 may also be referred to as the priming step. Priming may involve bringing the device into a condition in which it can be triggered. The second plunger boss (rib) 1040.2, 1040.2a, 1040.2b may abut the abutment surface 507b of the needle cover 500 after priming.

In order to the prime the drug delivery device 100, a force is applied to the needle cover 500 through the distal holes 208, e.g. with the help of an external tool that is pressed onto the distal end of the needle cover, such that the needle cover 500 is moved in the proximal direction. The proximal movement of the needle cover 500 brings the plunger boss second plunger boss (rib) 1040.2, 1040.2a, 1040.2b in engagement with the plunger boss slot 506, thus establishing a releasable lock between the needle cover 500 and the plunger 1000. Furthermore, the plunger 1000 rotates to its pre-use position in the drive spring holder 1200. The releasable lock between the needle cover 500 and the plunger 1000 prevents distal movement of the needle cover 500 relative to the device body 700, positions the needle cover 500 in the first cover position X, partially compresses the needle cover spring 600, and supports the needle cover 500 against the spring force of the needle cover spring 600. In addition, the proximal movement of the needle cover 500 changes the state of the locking between the plunger 1000 and the drive spring holder 1200.

Finally, the cap lid 300 is fitted onto the distal end of the cap 200. On the one hand, this causes the holes 208 to be closed and sealed and, on the other hand, the cap lid spacers 302 engage in the corresponding abutments, i.e. the cap-lid clips 209, of the cap 200 in order to provide the anti-recapping mechanism.

Similar drug delivery devices as the ones discussed above are described in WO2015/004049 A1, WO 2015/004052 A1, WO2015/144870 A1, WO2016/193374 A1, WO2016/193343 A1, WO2016/193375 A1, WO2016/193346 A1, WO2016/193348 A1, WO2016/193352 A1, WO2016/193353 A1, WO2016/193355 A1, WO2019/086561 A1, WO2019/086562 A1, WO2019/086575 A1, WO2019/086563 A1, WO2019/086564 A1, WO2019/086576 A1, WO2019/101613 A1, WO2020/069994 A1, WO2020/200995 A1, WO2020/245206 A1, WO2020/074570 A1, WO2022/003093 A1, WO2022/106504 A1, WO2017/089263 A1, WO2016/193344 A1, WO2016/193356 A1, WO2019/101689 A1 and WO2020/239844 A1, the entire disclosure content of each of which is included by reference for all purposes into the present disclosure.

Although embodiments of the present disclosure and their advantages have been described in detail, it should be understood that various changes, substitutions and alterations can be made therein without departing from the spirit and scope of the disclosure as defined by the appended claims. For example, it will be readily understood by those skilled in the art that many of the features, functions, processes and methods described herein may be varied while remaining within the scope of the present disclosure. Moreover, the scope of the present application is not intended to be limited to the particular embodiments of the system, process, manufacture, method or steps described in the present disclosure. As one of ordinary skill in the art will readily appreciate from the disclosure of the present disclosure, systems, processes, manufacture, methods or steps presently existing or to be developed later that perform substantially the same function or achieve substantially the same result as the corresponding embodiments described herein may be utilized according to the present disclosure. Accordingly, the appended claims are intended to include within their scope such systems, processes, methods or steps. The embodiments mentioned in the first part of the description may be combined with each other. The embodiments of the description of figures may also be combined with each other. Further, it is possible to combine embodiments mentioned in the first part of the description with examples of the second part of the description which relates to FIGS. 1A to 17.

### REFERENCE NUMERALS

A longitudinal axis
C cross axis
D distal direction
DE distal end
P proximal direction
PE proximal end
Dr drug
M medicament
100 drug delivery device
101 drive mechanism
102 rear sub-assembly
103 front sub-assembly
103.1 cap-gap
200 cap
201 cap body
202 gripping surface
203 cap-user-indicator
204 cap clip
205 anti-rotation ribs
206 cap opening
207 grabber retention bosses
207a proximal sloped section of boss
207b end surface of boss
207c connection region
208 distal holes
209 cap-lid clips
210 cap tube
211 grabber guide feature
212 inner distal holes
213 cap recess
213.1 distal recess end
213.2 proximal recess end
214 interference fit surfaces
300 cap lid
301 cap lid outer surface
302 cap lid spacer
303 positioning arc positioning guide
303a positioning guide
303b interference fit rib
304 positioning recess
400 grabber
402 grabber carrier
404 longitudinal fold edges
406 carrier portion
408 overlapping area
410 cut-out/opening
410a opening
410b retaining slot/opening
410c opening
412 barb
414 prong
416 orientation element
418 first/leading end
420 second/trailing end
422 oblique surface region
424 surface
426 surface
428 edge/corner
430 sheet
432 distal surface
434 cutout
436 indentations
D4 proximal portion of needle shield
D5 proximal portion of grabber
D6 distal portion of grabber
500 needle cover
501 skin contact surface
502 distal end portion
503 side region
503.1 lateral edge of side region
503a inner side region surface
503b outer side region surface
504 cap clip window
505 front stop slot
506 plunger boss slot
506a proximal slot
506b distal slot
507 slot rib
507a shoulder
507b abutment surface
507c first ramp
507d second ramp
508 plunger guide rib
509 grooves
510 flexible arm
510.1 cuboidal elevation
510.2 web
510.3 circular recess
513 proximal sleeve part
513.1 sleeve rib
513.2 proximal face
513.3 distal face
X first cover position
Y second cover position
Z third cover position
600 needle cover spring
601 double/triple winding
700 device body
700a sidewall
701 central support structure
702 central tube
703 axial support front end
703.1 conical inner surface
704 protrusion
705 syringe holder front stop
706 proximal syringe support
707 proximal syringe holder support
708 connecting rib
708a needle cover spring support
709 central support window
710 sidewall window
712 injection molding gate recess
713 distal cut-out
714 proximal cut-out
720 needle cover lock structure
720.1 needle cover lock disengagement prevention structure
721 needle cover back stop
722 needle cover radial support rib
723 needle cover guide rib
724 needle cover front stop
724.1 ramp-like section
724.2 cuboid section
725 cap groove
726 holder guide rib
730 proximal aperture
732 edge
733 body-user-indicator
800 syringe holder
800a holder housing
801 flexible holder arm
802 axial holder front end
803 holder protrusion
804 holder rear end
805 holder flange
806 holding clips
807 longitudinal rib
808 holder window
809 stop feature
810 ramp-like protrusion
811 guiding feature
812 recessed section
812a proximal end of recessed section
813 rounded section
813a proximal end of rounded section
814 support rib
816 distal portion of flexible holder arms
817 proximal portion of flexible holder arms
818 receiving space
819 inner surface rib
D7 distance distal end flexible arm to proximal end needle shield
I2 difference in axial extension between rounded and recessed section
900 pre-filled syringe
902 barrel
902OD outer diameter of barrel
904 shoulder
906 cone
908 needle
910 plunger stopper
912 syringe flange
914 rigid needle shield (RNS) or soft needle shield (SNS)
914OD outer diameter of needle shield
1000 plunger
1010 plunger shaft
1011d distal end of plunger
1011d proximal end of plunger
1012 plunger tip portion
1014 distal face
1016 annular face
1020, 1020a, 1020b plunger slot
1022, 1022a, 1022b plunger slot
1025 plunger release mechanism
1030 gap
1032 dashed line
1040.1, 1040.1a, 1040.1b first plunger boss (rib)
1040.1d distal edge (face)
1040.2, 1040.2a, 1040.2b second plunger boss (rib)
1040.3, 1040.3a, 1040.3b angled plunger rib
1040.9 proximal end of first plunger boss
R1 first rotational direction
R2 second rotational direction
1042a, 1042b pair of plunger ribs
TF triggering feature
1043 holding structure
1044 distal supporting portion (slanted portion)
1045 axially extending portion
1046 to 1049 rib
CR, CRa, CRb common rib
SR supporting rib
RF rounded supporting feature
1050a, 1050b groups of circumferential grooves
AS auxiliary structure
1051.1a, 1051.1b groove
1051.2a, 1051.2b groove
1051.3a, 1051.3b groove
1052a, 1052b pair of molding slots
1052.1a, 1052.2a slot
1052.1b, 1052.2b slot
1053 alternative molding opening(s)
1055 strongly slanted face
1056 moderately slanted face
1057, 1058 side face
1059 inner elongated cavity (hole)
IF interacting feature
1060 group of inner ribs
1060.1 to 1060.4 inner rib
1070 rounded chamfered portion
1071 outer surface
1072 outer edge
1073 proximal surface
1074 rounded edge
1075 slanted face
1076 rounded edge
1077 inner surface
1080 markings (indicia)
1080.1 to 1080.4 mark (indicium)
1082 further marking
1090 plastic material
TPP tool parting plane
RD radial direction
CD circumferential direction
1100 drive spring
1200 drive spring holder
1201 base
1201.1 proximal end surface
1202 syringe support arms
1202.1 guide ribs
1203 snap arms
1203.1 flexible part
1203.2 snap protrusion
1210 flexible portion
1211 distal end surface
1211.1 supporting rib
1212 flexible body
1212.1 chambers of flexible body
1212.2 connection structure of flexible body
1212.3 bent flexural beam
1221 proximal region
1221.1 profiled slot
1221.2 first angled surface
1221.3 wall
1221.4 second angled surface
1221.14 proximal wall of profiled slot
1221.15 indent
1221.16 ramp on indent
1230 spring support arm/pin
1234 inner longitudinal edge
1236 inner longitudinal rib
1238 gliding face
1239 proximal edge/face
1240 clicker support structure
1241 clicker support arm
1241.1 outwardly directed ramp-like structure
1241.1a, 1241.1b ramp
WP wall portion
SPPa, SPPb supporting portion
EP edge portion
G1 gap
G2 groove
1241.2 Inwardly directed ramp-like structure
1241.2a, 12412b ramp
1242 clicker tab restraint
FFa, FFb first face
SFa, SFb second face
TFa, TFb third face
1243 clicker rear support
R1 ramp
IP intermediate portion
R2 ramp
PP proximal portion
1244 clicker mounting groove
TP1a, TP1b lateral tab pre-assembling position
TP2a, TP2b lateral tab post-assembling position
1248 arrow (shift direction)
1250 indicator holder
1260 retaining cavity
BP bottom portion
BP1a, BP1b, BP1c first bottom portion (e.g., plane surface)
BP2 second bottom portion
SWa, SWb side wall
SWPa, SWPb side wall portion
AOa, AOb assembling opening
ARSa, ARSb auxiliary retaining space
FRSa, FRSb final retaining space
IRSa, IRSb intermediate retaining space
1270a, 1270b vertical rib
LPa, LPb lower portion
1280a, 1280b guiding structure
GFF1a to GFF5a face on guiding feature
1290a, 1290b raised portion
W2 to W4 angle
AR2 to AR6 arrow
DL1, DL2 dashed line
NCSFb1, NCSFb2 non coincident surface
web portion
Di1 to Di5 distance
1292 priming tool
1294 tip portion
1295 cylindrical portion
1296 tip holder
Ra1, Ra2 radius
AX axis
1299 arrow
RSA rear sub-assembly
FSA front sub-assembly
1300 clicker (audible indicator)
1301 resilient force member
1301a, 1301b wing-shaped section
S1, S2 conformation state
1302 plastically deformed bend (longitudinal round fold)
1303, 1303a, 1303b lateral (supporting) tab
1304 notch
1305d distal angled tab
1305p proximal angled tab
1312d distal end face
1312p proximal end face
1329 connecting portion
1330 main portion
1331 triangular portion
1341 to 1344 indentation
1350 elastically deformed bend
1361 to 1366 edge
1400 Press head (assembly process)
1410 proximal surface
1420 distal surface
1430 recess
1440 protrusions
D8 distal extension protrusion
F1 first feedback
F2 second feedback
F3 third feedback

1.-20. (canceled)

21. An assembly for a drug delivery device, the assembly comprising:
a device body having a proximal end and a distal end;
a needle cover that is movable relative to the device body along a longitudinal axis; and
a needle cover lock mechanism, comprising:
at least one blocking element defining a blocking surface; and
at least one flexible arm having a stop surface,
wherein the needle cover lock mechanism is configured to have a locked state, the stop surface and the blocking surface are arranged to cooperate to block movement of the needle cover in a proximal direction along the longitudinal axis when the needle cover lock mechanism is in the locked state, and the flexible arm comprises at least one protrusion positioned distally from the stop surface.

22. The assembly according to claim 21, wherein an angular extension of the stop surface is bigger than the angular extension of the protrusion.

23. The assembly according to claim 21, wherein a length of the protrusion along the longitudinal axis is shorter than the length of the flexible arm.

24. The assembly according to claim 21, wherein:
the needle cover is movable between an initial position and an intermediate position relative to the device body along the longitudinal axis,
the needle cover lock mechanism is in the locked state when the needle cover is in the initial position and in an unlocked state when the needle cover is in the intermediate position,
the assembly is configured such that the blocking element and the flexible arm overlap radially during movement from the initial position to the intermediate position and/or during movement from the intermediate position to the initial position.

25. The assembly according to claim 24, wherein the needle cover in the initial position is arranged more distally relative to the device body along the longitudinal axis than in the intermediate position.

26. The assembly according to claim 24, wherein the assembly is configured such that the flexible arm is moved radially inward when the radial overlap between the blocking element and the flexible arm begins during the movement from the intermediate position to the initial position.

27. The assembly according to claim 21, wherein:
the blocking element comprises a blocking element recess, and
the blocking element recess is configured such that a twisting of the flexible arm is prevented when the blocking element and the flexible arm radially overlap.

28. The assembly according to claim 27, wherein the needle cover lock mechanism is configured such that the protrusion is guided in the blocking element recess during movement from an intermediate position to an initial position.

29. The assembly according to claim 27, wherein the blocking element is intersected by the blocking element recess in the longitudinal direction.

30. The assembly according to claim 21, wherein:
the blocking element, defines two blocking surfaces angularly offset from each other, and
the two blocking surfaces have the same axial position relative to the device body.

31. The assembly according to claim 30, wherein an angular extension of the stop surface, corresponds to that of the angular extension of the two blocking surfaces and the blocking element recess together.

32. The assembly according to claim 21, wherein the blocking element has a ramp shape having a radially inward inclination.

33. The assembly according to claim 21, wherein the flexible arm comprises a hinge, and wherein the protrusion is positioned between the hinge and the stop surface in the longitudinal direction.

34. The assembly according to claim 21, wherein the protrusion and the stop surface extend radially outward from the flexible arm.

35. The assembly according to claim 21, wherein the assembly comprises two needle cover lock mechanisms which are arranged on the same axial position with an angular offset.

36. The assembly according to claim 21, wherein the protrusion is joined to the stop surface by a T-joint.

37. A drug delivery device comprising:
an assembly, comprising:
a device body having a proximal end and a distal end;
a needle cover that is movable relative to the device body along a longitudinal axis; and
a needle cover lock mechanism, comprising:
at least one blocking element defining a blocking surface; and
at least one flexible arm having a stop surface,
wherein the needle cover lock mechanism is configured to have a locked state,
the stop surface and the blocking surface are arranged to cooperate to block movement of the needle cover in a proximal direction along the longitudinal axis when the needle cover lock mechanism is in the locked state, and the flexible arm comprises at least one protrusion positioned distally from the stop surface.

38. The drug delivery device according to claim 37, comprising a container, wherein the container is prefilled with a drug.

39. A method of delivering a drug from a drug delivery device, the method comprising using the drug delivery device according to claim 37.

40. A drug for use in a method of treating a patient, wherein the method comprises delivering the drug to the patient using the drug delivery device according to claim 37.

41. A method of prophylactic treatment of a patient with hemophilia A or hemophilia B, with or without factor VIII or IX inhibitors, the method comprising:
subcutaneously delivering, using a drug delivery device, about 50 mg of fitusiran to the patient in need thereof once every two months, wherein the about 50 mg of fitusiran is delivered in about 0.5 mL of phosphate buffered saline (PBS) at a concentration of about 100 mg fitusiran/mL,
wherein the drug delivery device comprises
a device body having a proximal end and a distal end;
a needle cover that is movable relative to the device body along a longitudinal axis when the needle cover is in an unlocked state and is prevented from moving in a proximal direction along the longitudinal axis when the needle cover is in a locked state.

42. The method of claim 41, further comprising a needle cover lock mechanism, the needle cover lock mechanism comprising:
at least one blocking element defining a blocking surface; and
at least one flexible arm having a stop surface,
wherein the stop surface and the blocking surface are arranged to cooperate to block movement of the needle cover in the proximal direction along the longitudinal axis when the needle cover is in the locked state, and the flexible arm comprises at least one protrusion positioned distally from the stop surface.

43. A method of reducing frequency of bleeding episodes in a patient with hemophilia A or hemophilia B, with or without factor VIII or IX inhibitors, the method comprising:
subcutaneously delivering, using a drug delivery device, about 50 mg of fitusiran to the patient in need thereof once every two months, wherein the about 50 mg of fitusiran is delivered in about 0.5 mL of phosphate buffered saline (PBS) at a concentration of about 100 mg fitusiran/mL,
wherein the drug delivery device comprises
a device body having a proximal end and a distal end;
a needle cover that is movable relative to the device body along a longitudinal axis when the needle cover is in an unlocked state and is prevented from moving in a proximal direction along the longitudinal axis when the needle cover is in a locked state.

44. The method of claim 43, further comprising a needle cover lock mechanism, the needle cover lock mechanism comprising:
at least one blocking element defining a blocking surface; and
at least one flexible arm having a stop surface,
wherein the stop surface and the blocking surface are arranged to cooperate to block movement of the needle cover in the proximal direction along the longitudinal axis when the needle cover is in the locked state, and the flexible arm comprises at least one protrusion positioned distally from the stop surface.
